SEQUENCE LISTING (21 sequences)

SEQ ID NO: 1 - DNA, 3260 bp, Mus musculus
  CDS (486)..(2501)
tacactccttgccacagtctggcagaaagaatcaaacttcagagactcctccggccagttgtagacactatccttgtcaagtgtgcagatccaacagccgcacgagtcagctgtccatatctacagtgctggaactctgcaagggccaagcaggagagctggcggttgggagagaaatacttaaagctgggtccatcggggttggtggtgtcgattacgtcttaagttgtatccttggaaaccaagctgaatcaaacaactggcaagaactgctgggtcgcctctgtcttatagacaggttgctgttggaatttcctgctgaattctatcctcatattgtcagtactgatgtctcacaagctgagcctgttgaaatcaggtacaagaagctgctctccctcttaacctttgccttgcaatccattgacaattcccactcgatggttggcaagctctctcggaggatatatctgagctctgccaggatggtgaccgcagtgcccgctgtgttttccaagctggtaaccatgcttaatgcttctggctccacccacttcaccaggatgcgccggcgtctgatggctatcgcggatgaggtagaaattgccgaggtcatccagctgggtgtggaggacactgtggatgggcatcaggacagcttacaggccgtggcccccaccagctgtctagaaaacagctcccttgagcacacagtccatagagagaaaactggaaaaggactaagtgctacgagactgagtgccagctcggaggacatttctgacagactggccggcgtctctgtaggacttcccagctcaacaacaacagaacaaccaaagccagcggttcaaacaaaaggcagaccccacagtcagtgtttgaactcctcccctttgtctcatgctcaattaatgttcccagcaccatcagccccttgttcctctgccccgtctgtcccagatatttctaagcacagaccccaggcatttgttccctgcaaaataccttccgcatctcctcagacacagcgcaagttctctctacaattccagaggaactgctctgaacaccgagactcagaccagctctccccagtcttcactcagtcaagacccccaccctccagtaacatacacaggccaaagccatcccgacccgttccgggcagtacaagcaaactaggggacgccacaaaaagtagcatgacacttgatctgggcagtgcttccaggtgtgacgacagctttggcggcggcggcaacagtggcaacgccgtcatacccagcgacgagacagtgttcacgccggtggaggacaagtgcaggttagatgtgaacaccgagctcaactccagcatcgaggaccttcttgaagcatccatgccttcaagtgacacgacagtcactttcaagtccgaagtcgccgtcctctctccggaaaaggccgaaaatgacgacacctacaaagacgacgtcaatcataatcaaaagtgcaaagaaaagatggaagctgaagaggaggaggctttagcgatcgccatggcgatgtcagcgtctcaggatgccctccccatcgtccctcagctgcaggtggaaaatggagaagatattatcatcattcagcaggacacaccagaaactcttccaggacataccaaagcgaaacagccttacagagaagacgctgagtggctgaaaggccagcagataggcctcggagcattttcttcctgttaccaagcacaggatgtggggactgggactttaatggctgtgaaacaggtgacgtacgtcagaaacacatcctccgagcaggaggaggtggtggaagcgttgagggaagagatccggatgatgggtcacctcaaccatccaaacatcatccggatgctgggggccacgtgcgagaagagcaactacaacctcttcattgagtggatggcgggaggatctgtggctcacctcttgagtaaatacggagctttcaaggagtcagtcgtcattaactacactgagcagttactgcgtggcctttcctatctccacgagaaccagatcattcacagagacgtcaaaggtgccaacctgctcattgacagcaccggtcagaggctgagaattgcagactttggagctgctgccaggttggcatcaaaaggaaccggtgcaggagagttccagggacagttactggggacaattgcattcatggcgcctgaggtcctaagaggtcagcagtatggtaggagctgtgatgtatggagtgttggctgcgccattatagaaatggcttgtgcaaaaccaccttggaatgcagaaaaacactccaatcatctcgccttgatatttaagattgctagcgcaactactgcaccgtccatcccgtcacacctgtccccgggtctgcgcgacgtggccgtgcgctgcttagaacttcagcctcaggaccggcctccgtccagagagctgctgaaacatccggtcttccgtaccacgtggtagttaattgttcagatcagctctaatggagacaggatatcgaaccgggagagagaaaagagaacttgtgggcgaccatgccgctaaccgcagccctcacgccactgaacagccagaaacggggccagcggggaaccgtacctaagcatgtgattgacaaatcatgacctgtacctaagctcgatatgcagacatctacagctcgtgcaggaactgcacaccgtgcctttcacaggactggctctgggggaccaggaaggcgatggagtttgcatgactaaagaacagaagcataaatttatttttggagcactttttcagctaatcagtattaccatgtacatcaacatgcccgccacatttcaaactcagactgtcccagatgtcaagatccactgtgtttgagtttgtttgcagttccctcagcttgctggtaattgtggtgttttgttttcgatgcaaatgtgatgtaatattcttattttctttggatcaaagctggactgaaaattgtactgtgtaattatttttgtgtttttaatgttatttggtactcgaattgtaaataacgtctactgctgtttattccagtttctactacctcaggtgtcctatagatttttcttctaccaaagttcactctcagaatgaaattctacgtgctgtgtgactatgactcctaagacttccagggcttaagggctaactcctattagcaccttactatgtaagcaaatgctacaaaaaaaaaaaaaaaaa

SEQ ID NO: 2 - protein, 672 aa, Mus musculus
MVTAVPAVFSKLVTMLNASGSTHFTRMRRRLMAIADEVEIAEVIQLGVEDTVDGHQDSLQAVAPTSCLENSSLEHTVHREKTGKGLSATRLSASSEDISDRLAGVSVGLPSSTTTEQPKPAVQTKGRPHSQCLNSSPLSHAQLMFPAPSAPCSSAPSVPDISKHRPQAFVPCKIPSASPQTQRKFSLQFQRNCSEHRDSDQLSPVFTQSRPPPSSNIHRPKPSRPVPGSTSKLGDATKSSMTLDLGSASRCDDSFGGGGNSGNAVIPSDETVFTPVEDKCRLDVNTELNSSIEDLLEASMPSSDTTVTFKSEVAVLSPEKAENDDTYKDDVNHNQKCKEKMEAEEEEALAIAMAMSASQDALPIVPQLQVENGEDIIIIQQDTPETLPGHTKAKQPYREDAEWLKGQQIGLGAFSSCYQAQDVGTGTLMAVKQVTYVRNTSSEQEEVVEALREEIRMMGHLNHPNIIRMLGATCEKSNYNLFIEWMAGGSVAHLLSKYGAFKESVVINYTEQLLRGLSYLHENQIIHRDVKGANLLIDSTGQRLRIADFGAAARLASKGTGAGEFQGQLLGTIAFMAPEVLRGQQYGRSCDVWSVGCAIIEMACAKPPWNAEKHSNHLALIFKIASATTAPSIPSHLSPGLRDVAVRCLELQPQDRPPSRELLKHPVFRTTW

SEQ ID NO: 3 - DNA, 5253 bp, Mus musculus
  CDS (15)..(4496)
gcccgcgagagaaaatggcggcggcggcgggcgatcgcgcctcgtcgtcgggattcccgggcgccgcggcggcgagtcccgaggcgggcggcggcggcggaggaggaggagctctccagggaagcggcgcgcccgcagcgggcgcggcggggctgctgcgggagcctggcagcgcgggccgcgagcgcgcggactggcggcggcggcacgtgcgcaaagtgcggagtgtggagctggaccagctgccggagcagccgctcttcctcgccgccgcctcgccgccctgcccatctacttccccgtcgccggagcccgcggacgcggctgcaggagcgagtcgcttccagcccgcggcgggaccgccacccccgggagcggcgagtcgctgcggctcccactctgccgagctggcggccgcgcgggacagcggcgcccggagccccgcgggggcggagccgccctctgcagcggccccctccggtcgagagatggagaataaagaaaccctcaaaggactgcacaagatggaggatcgcccggaggagagaatgatccgggagaagctcaaggcgacctgtatgccggcctggaagcacgagtggttggagaggaggaacaggagaggccctgtggtggtgaagccaatccctattaaaggagatggatctgaagtgaataacttggcagctgagccccagggagagggccaggcaggttccgctgcaccagcccccaagggccgacgaagcccatctcctggcagctctccgtcagggcgctcggtgaagccggaatccccaggagtaagacggaaacgagtgtccccggtgcctttccagagtggcagaatcacaccaccccgaagagccccatcaccggatggcttctccccgtacagcccagaggagacgagccgccgcgtgaacaaagtgatgagagccaggctgtacctgctgcagcagataggacccaactctttcctgattggaggagacagtccagacaataaataccgggtgtttattgggccacagaactgcagctgtgggcgtggagcattctgtattcacctcttgtttgtcatgctccgggtgtttcagctagaaccctctgaccccatgttatggagaaaaactttaaaaaatttcgaggttgagagtttgttccagaaataccacagtaggcgtagctcgagaatcaaagctccatcccggaacaccatccagaagtttgtgtcacgcatgtcaaattctcacacactgtcatcgtctagcacatccacatctagttcagaaaacagcatcaaggatgaagaggagcagatgtgtcccatctgcttgctgggcatgctggatgaggagagcctgactgtgtgtgaagatggctgcaggaacaagctgcaccaccattgcatgtccatctgggcggaagagtgtagaagaaatagagagcctttaatatgtcccctttgtagatctaagtggagatcccatgacttctacagccatgagttatcaagccccgtggagtcccccgcctccctgcgagctgtccagcagccatcctccccgcagcagcccgtggccggatcacagcggaggaatcaggagagcagttttaaccttactcattttggaacccagcagattccttccgcttacaaagatttggccgagccatggattcaggtgtttggaatggaactcgttggctgcttattctctagaaactggaacgtaagggaaatggcccttaggcgtctttcccacgacgttagtggggccctgttgttggcaaacggggagagcactggaaactctggaggcggcagtgggggcagcttaagcgcgggagcggccagcgggtcctcccagcccagcatctcaggggatgtggtggaggcgtgctgcagtgtcctgtctatagtctgcgctgaccctgtctacaaagtgtacgttgctgctttaaaaacattgagagccatgctggtatacactccttgccacagtctggcagaaagaatcaaacttcagagactcctccggccagttgtagacactatccttgtcaagtgtgcagatgccaacagccgcacgagtcagctgtccatatctacagtgctggaactctgcaagggccaagcaggagagctggcggttgggagagaaatacttaaagctgggtccatcggggttggtggtgtcgattacgtcttaagttgtatccttggaaaccaagctgaatcaaacaactggcaagaactgctgggtcgcctctgtcttatagacaggttgctgttggaatttcctgctgaattctatcctcatattgtcagtactgatgtctcacaagctgagcctgttgaaatcaggtacaagaagctgctctccctcttaacctttgccttgcaatccattgacaattcccactcgatggttggcaagctctctcggaggatatatctgagctctgccaggatggtgaccgcagtgcccgctgtgttttccaagctggtaaccatgcttaatgcttctggctccacccacttcaccaggatgcgccggcgtctgatggctatcgcggatgaggtagaaattgccgaggtcatccagctgggtgtggaggacactgtggatgggcatcaggacagcttacaggccgtggcccccaccagctgtctagaaaacagctcccttgagcacacagtccatagagagaaaactggaaaaggactaagtgctacgagactgagtgccagctcggaggacatttctgacagactggccggcgtctctgtaggacttcccagctcaacaacaacagaacaaccaaagccagcggttcaaacaaaaggcagaccccacagtcagtgtttgaactcctcccctttgtctcatgctcaattaatgttcccagcaccatcagccccttgttcctctgccccgtctgtcccagatatttctaagcacagaccccaggcatttgttccctgcaaaataccttccgcatctcctcagacacagcgcaagttctctctacaattccagaggaactgctctgaacaccgagactcagaccagctctccccagtcttcactcagtcaagacccccaccctccagtaacatacacaggccaaagccatcccgacccgttccgggcagtacaagcaaactaggggacgccacaaaaagtagcatgacacttgatctgggcagtgcttccaggtgtgacgacagctttggcggcggcggcaacagtggcaacgccgtcatacccagcgacgagacagtgttcacgccggtggaggacaagtgcaggttagatgtgaacaccgagctcaactccagcatcgaggaccttcttgaagcatccatgccttcaagtgacacgacagtcactttcaagtccgaagtcgccgtcctctctccggaaaaggccgaaaatgacgacacctacaaagacgacgtcaatcataatcaaaagtgcaaagaaaagatggaagctgaagaggaggaggctttagcgatcgccatggcgatgtcagcgtctcaggatgccctccccatcgtccctcagctgcaggtggaaaatggagaagatattatcatcattcagcaggacacaccagaaactcttccaggacataccaaagcgaaacagccttacagagaagacgctgagtggctgaaaggccagcagataggcctcggagcattttcttcctgttaccaagcacaggatgtggggactgggactttaatggctgtgaaacaggtgacgtacgtcagaaacacatcctccgagcaggaggaggtggtggaagcgttgagggaagagatccggatgatgggtcacctcaaccatccaaacatcatccggatgctgggggccacgtgcgagaagagcaactacaacctcttcattgagtggatggcgggaggatctgtggctcacctcttgagtaaatacggagctttcaaggagtcagtcgtcattaactacactgagcagttactgcgtggcctttcctatctccacgagaaccagatcattcacagagacgtcaaaggtgccaacctgctcattgacagcaccggtcagaggctgagaattgcagactttggagctgctgccaggttggcatcaaaaggaaccggtgcaggagagttccagggacagttactggggacaattgcattcatggcgcctgaggtcctaagaggtcagcagtatggtaggagctgtgatgtatggagtgttggctgcgccattatagaaatggcttgtgcaaaaccaccttggaatgcagaaaaacactccaatcatctcgccttgatatttaagattgctagcgcaactactgcaccgtccatcccgtcacacctgtccccgggtctgcgcgacgtggccgtgcgctgcttagaacttcagcctcaggaccggcctccgtccagagagctgctgaaacatccggtcttccgtaccacgtggtagttaattgttcagatcagctctaatggagacaggatatgcaaccgggagagagaaaagagaacttgtgggcgaccatgccgctaaccgcagccctcacgccactgaacagccagaaacggggccagcggggaaccgtacctaagcatgtgattgacaaatcatgacctgtacctaagctcgatatgcagacatctacagctcgtgcaggaactgcacaccgtgcctttcacaggactggctctgggggaccaggaaggcgatggagtttgcatgactaaagaacagaagcataaatttatttttggagcactttttcagctaatcagtattaccatgtacatcaacatgcccgccacatttcaaactcagactgtcccagatgtcaagatccactgtgtttgagtttgtttgcagttccctcagcttgctggtaattgtggtgttttgttttcgatgcaaatgtgatgtaatattcttattttctttggatcaaagctggactgaaaattgtactgtgtaattatttttgtgtttttaatgttatttggtactcgaattgtaaataacgtctactgctgtttattccagtttctactacctcaggtgtcctatagatttttcttctaccaaagttcactctcagaatgaaattctacgtgctgtgtgactatgactcctaagacttccagggcttaagggctaactcctattagcaccttactatgtaagcaaatgctacaaaaaaaaaaaaaaaaaa

SEQ ID NO: 4 - protein, 1493 aa, Mus musculus
MAAAAGDRASSSGFPGAAAASPEAGGGGGGGGALQGSGAPAAGAAGLLREPGSAGRERADWRRRHVRKVRSVELDQLPEQPLFLAAASPPCPSTSPSPEPADAAAGASRFQPAAGPPPPGAASRCGSHSAELAAARDSGARSPAGAEPPSAAAPSGREMENKETLKGLHKMEDRPEERMIREKLKATCMPAWKHEWLERRNRRGPVVVKPIPIKGDGSEVNNLAAEPQGEGQAGSAAPAPKGRRSPSPGSSPSGRSVKPESPGVRRKRVSPVPFQSGRITPPRRAPSPDGFSPYSPEETSRRVNKVMRARLYLLQQIGPNSFLIGGDSPDNKYRVFIGPQNCSCGRGAFCIHLLFVMLRVFQLEPSDPMLWRKTLKNFEVESLFQKYHSRRSSRIKAPSRNTIQKFVSRMSNSHTLSSSSTSTSSSENSIKDEEEQMCPICLLGMLDEESLTVCEDGCRNKLHHHCMSIWAEECRRNREPLICPLCRSKWRSHDFYSHELSSPVESPASLRAVQQPSSPQQPVAGSQRRNQESSFNLTHFGTQQIPSAYKDLAEPWIQVFGMELVGCLFSRNWNVREMALRRLSHDVSGALLLANGESTGNSGGGSGGSLSAGAASGSSQPSISGDVVEACCSVLSIVCADPVYKVYVAALKTLRAMLVYTPCHSLAERIKLQRLLRPVVDTILVKCADANSRTSQLSISTVLELCKGQAGELAVGREILKAGSIGVGGVDYVLSCILGNQAESNNWQELLGRLCLIDRLLLEFPAEFYPHIVSTDVSQAEPVEIRYKKLLSLLTFALQSIDNSHSMVGKLSRRIYLSSARMVTAVPAVFSKLVTMLNASGSTHFTRMRRRLMAIADEVEIAEVIQLGVEDTVDGHQDSLQAVAPTSCLENSSLEHTVHREKTGKGLSATRLSASSEDISDRLAGVSVGLPSSTTTEQPKPAVQTKGRPHSQCLNSSPLSHAQLMFPAPSAPCSSAPSVPDISKHRPQAFVPCKIPSASPQTQRKFSLQFQRNCSEHRDSDQLSPVFTQSRPPPSSNIHRPKPSRPVPGSTSKLGDATKSSMTLDLGSASRCDDSFGGGGNSGNAVIPSDETVFTPVEDKCRLDVNTELNSSIEDLLEASMPSSDTTVTFKSEVAVLSPEKAENDDTYKDDVNHNQKCKEKMEAEEEEALAIAMAMSASQDALPIVPQLQVENGEDIIIIQQDTPETLPGHTKAKQPYREDAEWLKGQQIGLGAFSSCYQAQDVGTGTLMAVKQVTYVRNTSSEQEEVVEALREEIRMMGHLNHPNIIRMLGATCEKSNYNLFIEWMAGGSVAHLLSKYGAFKESVVINYTEQLLRGLSYLHENQIIHRDVKGANLLIDSTGQRLRIADFGAAARLASKGTGAGEFQGQLLGTIAFMAPEVLRGQQYGRSCDVWSVGCAIIEMACAKPPWNAEKHSNHLALIFKIASATTAPSIPSHLSPGLRDVAVRCLELQPQDRPPSRELLKHPVFRTTW

SEQ ID NO: 5 - DNA, 3911 bp, Homo sapiens
  CDS (3)..(3911)
cggcctggaagcacgagtggttggaaaggagaaataggcgagggcctgtggtggtaaaaccaatcccagttaaaggagatggatctgaaatgaatcacttagcagctgagtctccaggagaggtccaggcaagtgcggcttcaccagcttccaaaggccgacgcagtccttctcctggcaactccccatcaggtcgcacagtgaaatcagaatctccaggagtaaggagaaaaagagtttccccagtgccttttcagagtggcagaatcacaccaccccgaagagccccttcaccagatggcttctcaccatatagccctgaggaaacaaaccgccgtgttaacaaagtgatgcgggccagactgtacttactgcagcagatagggcctaactctttcctgattggaggagacagcccagacaataaataccgggtgtttattgggcctcagaactgcagctgtgcacgtggaacattctgtattcatctgctatttgtgatgctccgggtgtttcaactagaaccttcagacccaatgttatggagaaaaactttaaagaattttgaggttgagagtttgttccagaaatatcacagtaggcgtagctcaaggatcaaagctccatctcgtaacaccatccagaagtttgtttcacgcatgtcaaattctcatacattgtcatcatctagtacttctacatctagttcagtaaacagcataaaggatgaagaggaacagatgtgtcctatttgcttgttgggcatgcttgatgaagaaagtcttacagtgtgtgaagacggctgcaggaacaagctgcaccaccactgcatgtcaatttgggcagaagagtgtagaagaaatagagaacctttaatatgtcccctttgtagatctaagtggagatctcatgatttctacagccacgagttgtcaagtcctgtggattccccttcttccctcagagctgcacagcagcaaaccgtacagcagcagcctttggctggatcacgaaggaatcaagagagcaattttaaccttactcattatggaactcagcaaatccctcctgcttacaaagatttagctgagccatggattcaggtgtttggaatggaactcgttggctgcttattctctagaaactggaacgtaagggaaatggcccttaggcgtctttcccacgacgttagtggggccctgttgttggcaaacggggagagcactggaaactctggaggcggcagtgggggcagcttaagcgcgggagcggccagcgggtcctcccagcccagcatctcaggggatgtggtggaggcgtgctgcagtgtcctgtctatagtctgcgctgaccctgtctacaaagtgtacgttgctgctttaaaaacattgagagccatgctggtatacactccttgccacagtctggcagaaagaatcaaacttcagagactcctccggccagttgtagacactatccttgtcaagtgtgcagatgccaacagccgcacgagtcagctgtccatatctacagtgctggaactctgcaatggccaagcaggaaagctggcggttgggagagaaatacttaaagctgggtccatcggggttggtggtgtcgattacgtcttaagttgtatccttggaaaccaagctgaatcaaacaactggcaagaactgctgggtcgcctctgtcttatagacaggttgctgttggaatttcctgctgaattctatcctcatattgtcagtactgatgtctcacaagctgagcctgttgaaatcaggtacaagaagctgctctccctcttaacctttgccttgcaatccattgacaattcccactcgatggttggcaagctctctcggaggatatatctgagctctgccaggatggtgaccgcagtgcccgctgtgttttccaagctggtaaccatgcttaatgcttctggctccacccacttcaccaggatgcgccggcgtctgatggctatcgcggatgaggtagaaattgccgaggtcatccagctgggtgtggaggacactgtggatgggcatcaggacagcttacaggcgctggcccccgccagctgtctagaaaacagctcccttgagcacacagtccatagagagaaaactggaaaaggactaagtgctacgagactgagtgccagctcggaggacatttctgacagactggccggcgtctctgtaggacttcccagctcaacaacaacagaacaaccaaagccagcggttcaaacaaaaggcagaccccacagtcagtgtttgaactcctcccctttgtctcatgctcaattaatgttcccagcaccatcagccccttgttcctctgccccgtctgtcccagatatttctaagcacagaccccaggcatttgttccctgcaaaataccttccgcatctcctcagacacagcgcaagttctctctacaattccagaggaactgctctgaacaccgagactcagaccagctctccccagtcttcactcagtcaagacccccaccctccagtaacatacacaggccaaagccatcccgacccgttccgggcagtacaagcaaactaggggacgccacaaaaagtagcatgacacttgatctgggcagtgcttccaggtgtgacgacagctttggcggcggcggcaacagtggcaacgccgtcatacccagcgacgagacagtgttcacgccggtggaggacaagtgcaggttagatgtgaacaccgagctcaactccagcatcgaggaccttcttgaagcatccatgccttcaagtgacacgacagtcactttcaagtccgaagtcgccgtcctctctccggaaaaggccgaaaatgacgacacctacaaagacgacgtcaatcataatcaaaagtgcaaagaaaagatggaagctgaagaggaggaggctttagcgatcgccatggcgatgtcagcgtctcaggatgccctccccatcgtccctcagctgcaggtggaaaatggagaagatattatcatcattcagcaggacacaccagaaactcttccaggacataccaaagcgaaacagccttacagagaagacgctgagtggctgaaaggccagcagataggcctcggagcattttcttcttgttatcaggctcaagatgtgggaactggaactttaatggctgttaaacaggtgacttatgtcagaaacacatcttctgagcaagaagaagtagtagaagcactaagagaagagataagaatgatgagccatctgaatcatccaaacatcattaggatgttgggagccacgtgtgagaagagcaattacaatctcttcattgaatggatggcagggggatcggtggctcatttgctgagtaaatatggagccttcaaagaatcagtagttattaactacactgaacagttactccgtggcctttcgtatctccatgagaaccagatcattcacagagatgtcaaaggtgccaatttgctcattgacagcaccggtcagaggctgagaattgcagactttggagctgcagccaggttggcatcaaaaggaactggtgcaggagagtttcagggacaattactggggacaattgcattcatggcgcctgaggtcctaagaggtcagcagtatggtaggagctgtgatgtatggagtgttggctgcgccattatagaaatggcttgtgcaaaaccaccttggaatgcagaaaaacactccaatcatctcgccttgatatttaagattgctagcgcaactactgcaccgtccatcccgtcacacctgtcccctggtttacgagatgtggctcttcgttgtttagaacttcagcctcaggaccggcctccgtcaagagagctgctgaaacatccggtcttccgtaccacgtggtag

SEQ ID NO: 6 - protein, 1302 aa, Homo sapiens
AWKHEWLERRNRRGPVVVKPIPVKGDGSEMNHLAAESPGEVQASAASPASKGRRSPSPGNSPSGRTVKSESPGVRRKRVSPVPFQSGRITPPRRAPSPDGFSPYSPEETNRRVNKVMRARLYLLQQIGPNSFLIGGDSPDNKYRVFIGPQNCSCARGTFCIHLLFVMLRVFQLEPSDPMLWRKTLKNFEVESLFQKYHSRRSSRIKAPSRNTIQKFVSRMSNSHTLSSSSTSTSSSVNSIKDEEEQMCPICLLGMLDEESLTVCEDGCRNKLHHHCMSIWAEECRRNREPLICPLCRSKWRSHDFYSHELSSPVDSPSSLRAAQQQTVQQQPLAGSRRNQESNFNLTHYGTQQIPPAYKDLAEPWIQVFGMELVGCLFSRNWNVREMALRRLSHDVSGALLLANGESTGNSGGGSGGSLSAGAASGSSQPSISGDVVEACCSVLSIVCADPVYKVYVAALKTLRAMLVYTPCHSLAERIKLQRLLRPVVDTILVKCADANSRTSQLSISTVLELCNGQAGKLAVGREILKAGSIGVGGVDYVLSCILGNQAESNNWQELLGRLCLIDRLLLEFPAEFYPHIVSTDVSQAEPVEIRYKKLLSLLTFALQSIDNSHSMVGKLSRRIYLSSARMVTAVPAVFSKLVTMLNASGSTHFTRMRRRLMAIADEVEIAEVIQLGVEDTVDGHQDSLQALAPASCLENSSLEHTVHREKTGKGLSATRLSASSEDISDRLAGVSVGLPSSTTTEQPKPAVQTKGRPHSQCLNSSPLSHAQLMFPAPSAPCSSAPSVPDISKHRPQAFVPCKIPSASPQTQRKFSLQFQRNCSEHRDSDQLSPVFTQSRPPPSSNIHRPKPSRPVPGSTSKLGDATKSSMTLDLGSASRCDDSFGGGGNSGNAVIPSDETVFTPVEDKCRLDVNTELNSSIEDLLEASMPSSDTTVTFKSEVAVLSPEKAENDDTYKDDVNHNQKCKEKMEAEEEEALAIAMAMSASQDALPIVPQLQVENGEDIIIIQQDTPETLPGHTKAKQPYREDAEWLKGQQIGLGAFSSCYQAQDVGTGTLMAVKQVTYVRNTSSEQEEVVEALREEIRMMSHLNHPNIIRMLGATCEKSNYNLFIEWMAGGSVAHLLSKYGAFKESVVINYTEQLLRGLSYLHENQIIHRDVKGANLLIDSTGQRLRIADFGAAARLASKGTGAGEFQGQLLGTIAFMAPEVLRGQQYGRSCDVWSVGCAIIEMACAKPPWNAEKHSNHLALIFKIASATTAPSIPSHLSPGLRDVALRCLELQPQDRPPSRELLKHPVFRTTW

SEQ ID NO: 7 - protein, 4 aa, synthetic construct
DEVE

SEQ ID NO: 8 - protein, 4 aa, synthetic construct
DTVD

SEQ ID NO: 9 - protein, 15 aa, synthetic construct
DRPPSRELLKHPVER

SEQ ID NO: 10 - protein, 4 aa, synthetic construct
YVAD

SEQ ID NO: 11 - protein, 4 aa, synthetic construct
DEVD

SEQ ID NO: 12 - protein, 11 aa, synthetic construct
MGYPYDVDYAS

SEQ ID NO: 13 - DNA, 20 bp, synthetic construct
gaacaccatccagaagtttg

SEQ ID NO: 14 - DNA, 21 bp, synthetic construct
cactttgtagacagggtcagc

SEQ ID NO: 15 - DNA, 25 bp, synthetic construct
tgggtcgcctctgtcttatagacag

SEQ ID NO: 16 - DNA, 20 bp, synthetic construct
cacatcctgtgcttggtaac

SEQ ID NO: 17 - DNA, 21 bp, synthetic construct
aggacaagtgcaggttagatg

SEQ ID NO: 18 - DNA, 21 bp, synthetic construct
gctgtccatatctacagtgct

SEQ ID NO: 19 - DNA, 21 bp, synthetic construct
cggcctggaagcacgagtggt

SEQ ID NO: 20 - DNA, 21 bp, synthetic construct
ttcatccttgatgctgttttc

SEQ ID NO: 21 - protein, 1493 aa, Rattus norvegicus
MAAAAGDRASSSGFPGAAAASPEAGGGGGALQGSGAPAAGAGLLRETGSAGRERADWRRQQLRKVRSVELDQLPEQPLFLTASPPCPSTSPSPEPADAAAGASGFQPAAGPPPPGAASRCGSHSAELAAARDSGARSPAGAEPPSAAAPSGREMENKETLKGLHKMDDRPEERMIREKLKATCMPAWKHEWLERRNRRGPVVVKPIPIKGDGSEMSNLAAELQGEGQAGSAAPAPKGRRSPSPGSSPSGRSGKPESPGVRRKRVSPVPFQSGRITPPRRAPSPDGFSPYSPEETSRRVNKVMRARLYLLQQIGPNSFLIGGDSPDNKYRVFIGPQNCSCGRGTFCIHLLFVMLRVFQLEPSDPMLWRKTLKNFEVESLFQKYHSRRSSRIKAPSRNTIQKFVSRMSNCHTLSSSSTSTSSSENSIKDEEEQMCPICLLGMLDEESLTVCEDGCRNKLHHHCMSIWAEECRRNREPLICPLCRSKWRSHDFYSHELSSPVDSPTSLRGVQQPSSPQQPVAGSQRRNQESNFNLTHYGTQQIPPAYKDLAEPWIQAFGMELVGCLFSRNWNVREMALRRLSHDVSGALLLANGESTGTSGGGSGGSLSAGAASGSSQPSISGDVVEAFCSVLSIVCADPVYKVYVAALKTLRAMLVYTPCHSLAERIKLQRLLRPVVDTILVKCADANSRTSQLSISTLLELCKGQAGELAVGREILKAGSIGVGGVDYVLSCILGNQAESNNWQELLGRLCLIDRLLLEISAEFYPHIVSTDVSQAEPVEIRYKKLLSLLAFALQSIDNSHSMVGKLSRRIYLSSARMVTTVPPLFSKLVTMLSASGSSHFARMRRRLMAIADEVEIAEVIQLGSEDTLDGQQDSSQALAPPRYPESSSLEHTAHVEKTGKGLKATRLSASSEDISDRLAGVSVGLPSSATTEQPKPTVQTKGRPHSQCLNSSPLSPPQLMFPAISAPCSSAPSVPAGSVTDASKHRPRAFVPCKIPSASPQTQRKFSLQFQRTCSENRDSEKLSPVFTQSRPPPSSNIHRAKASRPVPGSTSKLGDASKNSMTLDLNSASQCDDSFGSGSNSGSAVIPSEETAFTPAEDKCRLDVNPELNSSIEDLLEASMPSSDTTVTFKSEVAVLSPEKAESDDTYKDDVNHNQKCKEKMEAEEEEALAIAMAMSASQDALPIVPQLQVENGEDIIIIQADTPETLPGHTKANEPYREDTEWLKGQQIGLGAFSSCYQAQDVGTGTLMAVKQVTYVRNTSSEQEEVVEALREEIRMMSHLNHPNIIRMLGATCEKSNYNLFIEWMAGASVAHLLSKYGAFKESVVINYTEQLLRGLSYLHENQIIHRDVKGANLLIDSTGQRLRIADFGAAARLASKGTGAGEFQGQLLGTIAFMAPEVLRGQQYGRSCDVWSVGCAIIEMACAKPPWNAEKHSNHLALIFKIASATTAPSIPSHLSPGLRDVALRCLELQPQDRPPSRELLKHPVFRTTW

(19) United States
(12) Patent Application Publication
(10) Pub. No.: US 2005/0244846 A1
(43) Pub. Date: Nov. 3, 2005

(54) MEKK1 proteins and fragments thereof for use in regulating apoptosis

(76) Inventor: Gary L. Johnson, Boulder, CO (US)

Correspondence Address: LAHIVE & COCKFIELD, LLP., 28 STATE STREET, BOSTON, MA, 02109, US

(21) Appl. No.: 11/004,057

(22) Filed: Dec. 2, 2004

Related U.S. Application Data
(63) Continuation of application No. 09/403,075, filed on May 10, 2000, now abandoned.
Application No. 09/403,075 is a continuation-in-part of application No. 09/023,130, filed on Feb. 13, 1998, now abandoned.
(60) Provisional application No. 60/039,740, filed on Feb. 14, 1997.

(51) Int. Cl. C12Q 1/68; C07H 21/04; C12N 9/12; C12N 15/09

(52) U.S. Cl. 435/6; 435/69.1; 435/194; 435/320.1; 435/325; 536/23.2

(57) ABSTRACT

The present invention relates to isolated MEKK1 proteins, nucleic acid molecules having sequences that encode such proteins, and antibodies raised against such proteins. The present invention also includes methods to use such proteins to regulate apoptosis. The invention provides active fragments of MEKK1 proteins that are generated upon cleavage of MEKK1 with a caspase protease. These active fragments are capable of stimulating apoptosis. Moreover, the invention provides protease-resistant forms of MEKK1 proteins, that are resistant to cleavage by caspase proteases and that are capable of inhibiting apoptosis. Still further, the invention provides methods for generating an active fragment of MEKK1, methods of identifying modulators of the apoptotic activity of an active fragment of MEKK1 and methods of identifying modulators of caspase-mediated cleavage of MEKK1.

### RELATED APPLICATIONS

This application is a continuation of U.S. patent application Ser. No. 09/403,075, filed May 10, 2000, which is a U.S. National Phase Application of PCT/US99/02974, filed Feb. 12, 1999, which is a continuation-in-part of U.S. patent application Ser. No. 09/023,130, filed on Feb. 13, 1998. The contents of each of the aforementioned applications is expressly incorporated herein by reference.

### GOVERNMENT FUNDING

This invention was made in part with government support under USPHS Grants DK37871: DK48845, CA58157 and GM30324, each awarded by the National Institutes of Health. The government has certain rights in this invention.

### FIELD OF THE INVENTION

This invention relates to isolated nucleic acid molecules encoding MEKK1 proteins, substantially pure MEKK1 proteins, and products and methods for regulating apoptosis in cells.

### BACKGROUND OF THE INVENTION

Mitogen-activated protein kinase (MAPKs) (Mitogen-Activated Protein Kinases, also called extracellular signal-regulated kinases or ERKs) are rapidly activated in response to ligand binding by both growth factor receptors that are tyrosine kinases and receptors that are coupled to heterotrimeric guanine nucleotide binding proteins (G proteins). MAPKs integrate multiple intracellular signals transmitted by various second messengers via a mechanism which involves the phosphorylation and regulation of the activity of enzymes and transcription factors including the EGF receptor, Rsk 90, phospholipase A₂, c-Myc, c-Jun and Elk-1/TCF.

MAPKs are in turn phosphorylated and regulated by proteins called MEKs (MAPK Kinase or ERK Kinase) or MKK (MAP Kinase kinase). The MEKs phosphorylate MAPKs on both tyrosine and threonine residues which results in activation of MAPKs. MEKs are likewise phosphorylated and regulated by one of two distinct classes of mammalian serine-threonine protein kinase, the Rafs or the MEKKs (MEK Kinases).

Certain biological functions, such as growth and differentiation, are tightly regulated by signal transduction pathways within cells. Signal transduction pathways maintain the balanced steady state functioning of a cell. Disease states can arise when signal transduction in a cell breaks down, thereby removing the tight control that typically exists over cellular functions. For example, tumors develop when regulation of cell growth is disrupted, enabling a clone of cells to expand indefinitely. Because signal transduction networks regulate a multitude of cellular functions depending upon the cell type, a wide variety of diseases can result from abnormalities in such networks. Devastating diseases such as cancer, autoimmune diseases, allergic reactions, inflammation, neurological disorders and hormone-related diseases can result from abnormal signal transduction.

Given the importance of signal transduction molecules in regulating a variety of cellular processes and the important consequences of signal transduction aberrancies in disease states, there exists a need to identify novel signaling molecules. Moreover, understanding intracellular signaling pathways is advantageous in identifying and developing pharmacological and therapeutic agents targeted towards particular signaling molecules.

### SUMMARY OF THE INVENTION

The present invention is based, at least in part, on the identification of MEKK1 protein and nucleic acid molecules, in particular, human MEKK1 molecules, as well as bioactive fragments of MEKK1 molecules useful in regulating cellular apoptosis.

In one aspect, the present invention relates to isolated nucleic acid molecules having sequences that encode MEKK1 proteins, MEKK1 proteins, and antibodies raised against such proteins. In one embodiment, an isolated nucleic acid molecule of the invention comprises the nucleotide sequence shown in SEQ ID NO:3. The sequence of SEQ ID NO:3 corresponds to a murine MEKK1 cDNA. The predicted amino acid sequence of murine MEKK1 is set forth as SEQ ID NO 4. This cDNA comprises sequences encoding the murine MEKK1 protein (i.e., “the coding region”, from nucleotides 15-4496), as well as 5′ untranslated sequences (nucleotides 1-14) and 3′ untranslated sequences (nucleotides 4497-5253). The predicted amino acid sequence of murine MEKK1 is set forth as SEQ ID NO 4. In another embodiment, an isolated nucleic acid molecule of the invention comprises the nucleotide sequence shown in SEQ ID NO:5. The sequence of SEQ ID NO:5 corresponds to a human MEKK1 cDNA. This cDNA comprises sequences encoding human MEKK1 protein (i.e., a “coding region”, from nucleotides 3-3911). The predicted amino acid sequence of human MEKK1 is set forth as SEQ ID NO 5.

In another aspect, this invention provides isolated nucleic acid molecules encoding MEKK1 proteins or biologically active portions or fragments thereof (e.g., apoptotic portions or fragments), as well as nucleic acid fragments suitable as primers or hybridization probes for the detection of MEKK1-encoding nucleic acids.

In one embodiment, a MEKK1 nucleic acid molecule is 90% homologous to the nucleotide sequence shown in SEQ ID NO:3, SEQ ID NO:5, or complement thereof. In a preferred embodiment, an isolated MEKK nucleic acid molecule has the nucleotide sequence shown SEQ ID NO:3, or a complement thereof. In another embodiment, a MEKK nucleic acid molecule comprises nucleotides 15-4496 of SEQ ID NO:3. In another preferred embodiment, an isolated MEKK nucleic acid molecule has the nucleotide sequence shown in SEQ ID NO:5. In another embodiment, a MEKK1 nucleic acid molecule comprises nucleotides 3-3911 of SEQ ID NO:5.

In another embodiment, a MEKK1 nucleic acid molecule includes a nucleotide sequence encoding a protein having an amino acid sequence substantially homologous to the amino acid sequence of SEQ ID NO:4 or SEQ ID NO:6. In another preferred embodiment, a MEKK1 nucleic acid molecule includes a nucleotide sequence encoding a protein having an amino acid sequence at least 90% homologous to the amino acid sequence of SEQ ID NO:4 or SEQ ID NO:6. In yet another embodiment, a MEKK1 nucleic acid molecule is a naturally occurring nucleotide sequence (e.g., a naturally-occurring human or murine nucleotide sequence).

Another embodiment of the invention features isolated nucleic acid molecules which specifically detect MEKK1 nucleic acid molecules relative to nucleic acid molecules encoding non-MEKK1 proteins. For example, in one embodiment, an isolated nucleic acid molecule hybridizes under stringent conditions to a nucleic acid molecule comprising the nucleotide sequence shown in SEQ ID NO:3, SEQ ID NO:5, or a complement thereof. In another embodiment, an isolated nucleic acid molecule hybridizes to about nucleotides 1-2400 of SEQ ID NO:3. In another embodiment, an isolated nucleic acid molecule is at least 500 nucleotides in length and hybridizes under stringent conditions to a nucleic acid molecule comprising the nucleotide sequence shown in SEQ ID NO:3, SEQ ID NO:5, or a complement thereof

Another embodiment of the invention provides an isolated nucleic acid molecule which is antisense to the coding strand of a MEKK1 nucleic acid.

Another aspect of the invention provides a vector comprising a MEKK1 nucleic acid molecule. In certain embodiments, the vector is a recombinant expression vector. In another embodiment, the invention provides a host cell containing a vector of the invention. The invention also provides a method for producing a MEKK1 protein by culturing in a suitable medium, a host cell of the invention containing a recombinant expression vector such that a MEKK1 protein is produced.

Another aspect of this invention features isolated or recombinant MEKK1 proteins and polypeptides. In one embodiment, an isolated protein includes a biologically active portion of a MEKK1 protein (e.g., an apoptotic portion). In another embodiment, an isolated MEKK1 protein has an amino acid sequence substantially homologous to the amino acid sequence of SEQ ID NO:4 or SEQ ID NO:6. In a preferred embodiment, a MEKK1 protein has an amino acid sequence at least about 90% homologous to the amino acid sequence of SEQ ID NO:4 or SEQ ID NO:6. In another embodiment, a MEKK1 protein has the amino acid sequence of SEQ ID NO:4 or SEQ ID NO:6.

Another embodiment of the invention features an isolated MEKK1 protein which is encoded by a nucleic acid molecule having a nucleotide sequence at least about 90% homologous to a nucleotide sequence of SEQ ID NO:3, SEQ ID NO:5, or a complement thereof. This invention further features an isolated MEKK1 protein which is encoded by a nucleic acid molecule having a nucleotide sequence which hybridizes under stringent hybridization conditions to a nucleic acid molecule comprising the nucleotide sequence of SEQ ID NO:3, SEQ ID NO:5, or a complement thereof.

The MEKK1 proteins of the present invention, or biologically active portions thereof, can be operatively linked to a non-MEKK1 polypeptide (e.g., heterologous amino acid sequences) to form MEKK1 fusion proteins. The invention further features antibodies that specifically bind MEKK1 proteins, such as monoclonal or polyclonal antibodies. In addition, the MEKK1 proteins or biologically active portions thereof can be incorporated into pharmaceutical compositions, which optionally include pharmaceutically acceptable carriers.

In another aspect, the present invention provides a method for detecting the presence of a MEKK1 protein in a sample (e.g., biological sample) by contacting the sample with a compound which selectively binds to the protein and determining whether the compound binds to the protein in the sample to thereby detect the presence of a MEKK1 protein in the sample.

In another aspect, the present invention provides a method for detecting the presence of a MEKK1 nucleic acid molecule in a sample (e.g., biological sample) by contacting the sample with a nucleic acid probe or primer which selectively hybridizes to the nucleic acid molecule and determining whether the probe or primer binds to a nucleic acid molecule in the sample to thereby detect the presence of a MEKK1 nucleic acid molecule in the sample.

In another aspect, the present invention provides a method for detecting the presence of MEKK1 activity in a biological sample by contacting the biological sample with an agent capable of detecting MEKK1 activity such that the presence of MEKK1 activity is detected in the biological sample.

In another aspect, the invention provides a method for modulating MEKK1 activity comprising contacting the cell with an agent that modulates MEKK1 activity such that MEKK1 activity in the cell is modulated. In one embodiment, the agent inhibits MEKK1 activity. In another embodiment, the agent stimulates MEKK1 activity. In one embodiment, the agent is an antibody that specifically binds to a MEKK1 protein. In another embodiment, the agent modulates expression of MEKK1 by modulating transcription of a MEKK1 gene or translation of a MEKK1 mRNA. In yet another embodiment, the agent is a nucleic acid molecule having a nucleotide sequence that is antisense to the coding strand of a MEKK1 mRNA or a MEKK1 gene.

In one embodiment, the methods of the present invention are used to treat a subject having a disorder characterized by aberrant MEKK1 protein or nucleic acid expression or activity by administering an agent which is a MEKK1 modulator to the subject. In one embodiment, the MEKK1 modulator is a MEKK1 protein. In another embodiment the MEKK1 modulator is a MEKK1 nucleic acid molecule. In yet another embodiment, the MEKK1 modulator is a peptide, peptidomimetic, or other small molecule. In a preferred embodiment, the disorder characterized by aberrant MEKK1 protein or nucleic acid expression is a developmental, differentiative, proliferative. disorder, an immunological disorder, or cell death.

The present invention also provides a diagnostic assay for identifying the presence or absence of a genetic alteration characterized by at least one of (i) aberrant modification or mutation of a gene encoding a MEKK1 protein; (ii) mis-regulation of said gene; and (iii) aberrant post-translational modification of a MEKK1 protein, wherein a wild-type form of said gene encodes an protein with a MEKK1 activity.

The present invention also includes methods to use MEKK1 proteins to regulate apoptosis. The invention provides active fragments of MEKK1 proteins that are generated upon cleavage of MEKK1 with a caspase protease. These active fragments are capable of stimulating apoptosis. Moreover, the invention provides protease-resistant forms of MEKK1 proteins, that are resistant to cleavage by caspase proteases and that are capable of inhibiting apoptosis. Still further, the invention provides methods for generating an active fragment of MEKK1, methods of identifying modulators of the apoptotic activity of an active fragment of MEKK1 and methods of identifying modulators of caspase-mediated cleavage of MEKK1.

It has been discovered that MEK kinase 1 (MEKK1), a 196 kDa protein kinase, functions to integrate proteases and signal transduction pathways involved in the regulation of apoptosis. Cleavage of mouse MEKK1 at Asp⁸⁷⁴ generates a 91 kDa kinase fragment and a 113 kDa NH₂-terminal fragment. The kinase fragment of MEKK1 induces apoptosis. Cleavage of MEKK1 and apoptosis are inhibited by p35 and CrmA, viral inhibitors of the ICE/FLICE proteases that commit cells to apoptosis. Mutation of the MEKK1 sequence ⁸⁷¹DTVD⁸⁷⁴, a cleavage site for CCP32-like proteases, to alanines inhibited proteolysis of MEKK1 and apoptosis induced by overexpression of MEKK1. Inhibition of MEKK1 proteolysis inhibited apoptosis but did not block MEKK1 stimulation of c-Jun kinase activity, indicating that c-Jun kinase activation was not sufficient for apoptosis. During the apoptotic response to UV irradiation, cisplatin, etoposide and mitomycin C, MEKK1 undergoes a phosphorylation-dependent activation followed by its proteolysis. These results show that MEKK1 activation and cleavage occurs in response to genotoxic agents and the activated kinase fragment functions to commit cells to apoptosis.

Accordingly, this invention defines MEKK1 as a protease substrate that when activated and cleaved stimulates an apoptotic response. The proteolytic cleavage of MEKK1 defines the mechanism to generate a protein kinase whose activity is sufficient to induce apoptosis. In the context of cancer therapy, the finding that the activation and cleavage of MEKK1 occurs in response to genotoxic agents is particularly important. It has been found that expression of MEKK1 is capable of killing by apoptosis cells that have both p53 alleles mutated. Hence, the activation and cleavage of MEKK1 is an apoptotic pathway that does not require a functional p53 and stimulation of these events could enhance the killing of manydifferent tumors. Manipulating the activation of MEKK1 and its cleavage by proteases, with the use of drugs for example, could increase the killing of tumor cells to genotoxic agents. Consistent with this hypothesis is the finding that low level expression of MEKK1 potentiated the apoptotic response to low doses of UV irradiation and cisplatin.

One aspect of the present invention pertains to active fragments of MEKK1 proteins (i.e., fragments of MEKK1 proteins that retain apoptotic activity). Such active fragments can be generated naturally by cleavage of MEKK1 by a caspase protease. For example, an apoptotic fragment of murine MEKK1 can be generated by caspase after a cleavage site found at amino acids 871-874 of SEQ ID NO:4. Likewise, an apoptotic fragment of human MEKK1 can be generated by caspase after a cleavage site found at amino acids 681-684 of SEQ ID NO:6. Alternatively, the active fragments of the invention can be prepared by recombinant DNA technology, using standard methodologies. In one embodiment, the invention provides an isolated active fragment of an MEKK1 protein consisting of an amino acid sequence having at least 75% homology to an amino acid sequence consisting of about amino acids 875-1493 of SEQ ID NO:4, wherein said active fragment mediates apoptosis. Preferably, the active fragment consists of an amino acid sequence having at least 85% homology to an amino acid sequence consisting of about amino acids 875-1493 of SEQ ID NO:4. More preferably, the active fragment consists of an amino acid sequence having at least 95% homology to an amino acid sequence consisting of about amino acids 875-1493 of SEQ ID NO:4. In one embodiment, the active fragment is a mouse MEKK1 active fragment. In another embodiment, the active fragment is a human MEKK1 active fragment. In another embodiment, the active fragment is a rat MEKK1 active fragment. The active fragment can consist of, for example, about amino acids 875-1493 of SEQ ID NO:4. Preferably, the active fragment consists of amino acids 875-1493 of SEQ ID NO:4. The active fragment can consist of about amino acids 685-1303 of SEQ ID NO:6. Preferably, the active fragment consists of amino acids. 685-1303, of SEQ ID NO:4.

Another aspect of the invention pertains to protease-resistant forms of MEKK1 proteins. Such protease-resistant forms can be generated by mutation of the caspase cleavage site in an MEKK1 protein (e.g., a cleavage site corresponding to amino acids 871-874 of SEQ ID NO:4 or amino acids 681-684 of SEQ ID NO:6) such that the site cannot be cleaved by the caspase. Preferably, at least the Asp residue at 871 and/or 874 of SEQ ID NO:4 is mutated. Alternatively, at least the Asp residue at 681 and/or 684 of SEQ ID NO:6 is mutated. Preferably, one or more of the amino acids corresponding to 871-874 of SEQ ID NO:4 or to 681-684 of SEQ ID NO:6 can be mutated to, for example, alanine residues. Alternatively, said residue can be mutated to glutamine. Accordingly, the invention provides an isolated protease-resistant MEKK1 protein comprising an amino acid sequence having at least 75% homology to the amino acid sequence of SEQ ID NO:4, wherein at least one amino acid equivalent to amino acids 871-874 of SEQ ID NO:4 is substituted such that the MEKK1 protein is resistant to proteolysis by a caspase. Preferably, the protease-resistant MEKK1 protein has at least 85% homology to the amino acid sequence of SEQ ID NO:4. More preferably, the, protease-resistant MEKK1 protein has at least 95% homology to the amino acid sequence of SEQ ID NO:4. In one embodiment, the protease-resistant MEKK1 protein is a mouse MEKK1 protein. In another embodiment, the protease-resistant MEKK1 protein is a human MEKK1 protein. In yet another embodiment, the protease-resistant MEKK1 protein is a rat MEKK1 protein.

The invention farther provides isolated nucleic acid molecules that encode the MEKK1 active fragments of the invention. In one embodiment, the invention provides an isolated nucleic acid molecule consisting of a nucleotide sequence having at least 75% homology to a nucleotide sequence consisting of about nucleotides 2637-4493 of SEQ ID NO:3, wherein said nucleic acid molecule encodes an active fragment of MEKK1 that mediates apoptosis. Preferably, the nucleic acid molecule consists of a nucleotide sequence having at least 85% homology to a nucleotide sequence consisting of about nucleotides 2637-4493 of SEQ ID NO:3. More preferably, the nucleic acid molecule consists of a nucleotide sequence having at least 95% homology to a nucleotide sequence consisting of about nucleotides 2637-4493 of SEQ ID NO:3. In one embodiment, the nucleic acid molecule encodes an active fragment of mouse MEKK1. In another embodiment, the nucleic acid molecule encodes an active fragment of human MEKK1. In yet another embodiment, the nucleic acid molecule encodes an active fragment of rat MEKK1. In a preferred embodiment, the nucleic acid molecule comprises at least about nucleotides 2637-4493 of SEQ ID NO:3, or a nucleotide sequence that, due to the degeneracy of the genetic code, encodes the same amino acid sequence as about nucleotides 2637-4493 of SEQ ID NO:3. In another preferred embodiment, the nucleic acid molecule comprises at least about nucleotides 2052-3908 of SEQ ID NO:5, or a nucleotide sequence that, due to the degeneracy of the genetic code, encodes the same amino acid sequence as nucleotides 2052-3908 of SEQ ID NO:5.

The invention also provides isolated nucleic acid molecules encoding the protease-resistant forms of MEKK1 of the invention. For example, the invention provides an isolated nucleic acid molecule encoding a protease-resistant MEKK1 protein, wherein the protease resistant MEKK1 protein comprises an amino acid sequence having at least 75% homology to the amino acid sequence of SEQ ID NO:4 and at least one codon of the nucleic acid molecule encoding an amino acid equivalent to at least one of amino acids 871-874 of SEQ ID NO:4 is mutated such the encoded MEKK1 protein is resistant to proteolysis by a caspase after an amino acid equivalent to amino acid 874 of SEQ ID NO:4. Preferably, the MEKK1 protein comprises an amino acid sequence having at least 85% homology to the amino acid sequence of SEQ ID NO:4. More preferably, the MEKK1 protein comprises an amino acid sequence having at least 95% homology to the amino acid sequence of SEQ ID NO:4. In one embodiment, the nucleic acid encodes a protease-resistant mouse MEKK1 protein. In another embodiment, the nucleic acid encodes a protease-resistant human MEKK1 protein. In yet another embodiment, the nucleic acid molecule encodes a protease-resistant rat MEKK1 protein. In a preferred embodiment, the nucleic acid has the nucleic acid sequence of SEQ ID NO:5 where at least one codon encoding one of amino acids 681-684 of SEQ ID NO:6 is mutated such the encoded MEKK1 protein is resistant to proteolysis by a caspase after an amino acid equivalent to amino acid 684 of SEQ ID NO:6.

Yet another aspect of the invention pertains to methods for modulating apoptosis. In one embodiment, the invention provides a method of stimulating apoptosis in a cell comprising introducing into the cell an expression vector encoding an MEKK1 active fragment of the invention such that MEKK1 active fragment is produced in the cell and apoptosis is stimulated. In another embodiment, the invention provides a method of inhibiting apoptosis in a cell comprising introducing into the cell an expression vector encoding a protease-resistant MEKK1 protein of the invention such that protease-resistant MEKK1 protein is produced in the cell and apoptosis is inhibited.

The invention also provides methods for generating MEKK1 active fragments in vitro. For example, an MEKK1 active fragment can be generated in vitro by:
contacting an MEKK1 protein in vitro with a caspase protease under proteolysis conditions; and
allowing the caspase protease to cleave the MEKK1 protein such that an MEKK1 active fragment is generated.

Preferably, the caspase protease is a caspase-3 protease. Alternatively, the caspase protease is a caspase-7 protease. Standard proteolysis conditions known in the art under which caspase proteases are known to be active can be used in the method of the invention.

Still another aspect of the invention pertains to methods for identifying modulators of apoptosis. In one embodiment, the invention provides a method of identifying a compound that modulates the apoptotic activity of an MEKK1 active fragment. The method comprises:

providing an indicator cell that comprises an MEKK1 active fragment of the invention;

contacting the indicator cell with a test compound; and

determining the effect of the test compound on the apoptotic activity of the MEKK1 active fragment in the indicator cell to thereby identify a compound that modulates the apoptotic activity of the MEKK1 active fragment.

The indicator cell may naturally express an MEKK1 active fragment or may be transfected with an expression vector that encodes the MEKK1 active fragment such that the active fragment is expressed in the cell. The effect of the test compound can be evaluated, for example, by measuring an apoptotic response in the cells, such as DNA fragmentation.

In another embodiment, the invention provides a method of identifying a compound that modulates the proteolytic cleavage of an MEKK1 protein by a caspase protease, comprising:

providing a reaction mixture that comprises an MEKK1 protein and a caspase protease;

contacting the reaction mixture with a test compound; and

determining the effect of the test compound on proteolytic cleavage of the MEKK1 protein by the caspase protease to thereby identify a compound that modulates the proteolytic cleavage of an MEKK1 protein by a caspaseiprotease.

Preferably, the caspase protease is a caspase-3 protease. Alternatively, the caspase protease is a caspase-7 protease. Standard proteolysis conditions known in the art under which caspase proteases are known to be active can be used in the method of the invention. The effect of the test compound on the proteolytic cleavage of MEKK1 can be evaluated by, for example, monitoring the generation of the 91 kD active fragment of MEKK1 (e.g., by detection of the 91 kD fragment using an anti-MEKK1 antibody, using standard techniques).

### BRIEF DESCRIPTION OF THE FIGURES

FIG. 1 depicts the cDNA sequence of human MEKK1. The nucleotide sequence corresponds to nucleic acids 1 to 3911 of SEQ ID NO:5.

FIG. 2 depicts the cDNA sequence of murine MEKK1. The nucleotide sequence corresponds to nucleic acids 1 to 5253 of SEQ ID NO:3.

FIG. 3 depicts an alignment of the amino acid sequences of murine MEKK1 (amino acids 1-1493 of SEQ ID NO:4 and human MEKK1 (amino acids 1-1303 of SEQ ID NO:6). The conserved caspase cleavage site is boxed. Amino acids which are unique as between murine and human MEKK1 are underlined.

FIG. 4 is a schematic representation of the HA-tagged mouse MEKK1 protein showing the regions (the numbers correspond to the position of the amino acids) used to generate the indicated antibodies. Also shown is the sequence (one letter code) between amino acids 853 and 888 of SEQ ID NO:4 where the tetrapeptides DEVE (SEQ ID NO: 7) and DTVD (SEQ ID NO: 8) (in bold) have been replaced with alanine residues in mutants DEVE→A and DTVD→A, respectively.

FIG. 5 is a schematic representation of the p35-inhibitable and p35-insensitive cleavage in the mouse MEKK1 protein. The letters A to D indicate the names of the cleavage products. The molecular weights were calculated from the migration of the.: markers in at least 2 different experiments.

FIG. 6 is a schematic diagram of a mechanistic model of MEKK1-induced apoptosis.

FIG. 7 depicts an alignment of the amino acid sequences of murine MEKK1 and rat MEKK1 (having Accession No. Q62925). The rat MEKK1 amino acid sequence is set forth as SEQ ID NO:21. The predicted caspase cleavage site in rat is boxed. A predicted rat apoptotic fragment begins after the cleavage site and comprises amino acid residues 875-1493 of SEQ ID NO:21.

FIG. 8 depicts an alignment of the amino acid sequences of murine MEKK1, rat MEKK1 and partial amino acid sequences of human MEKK1.

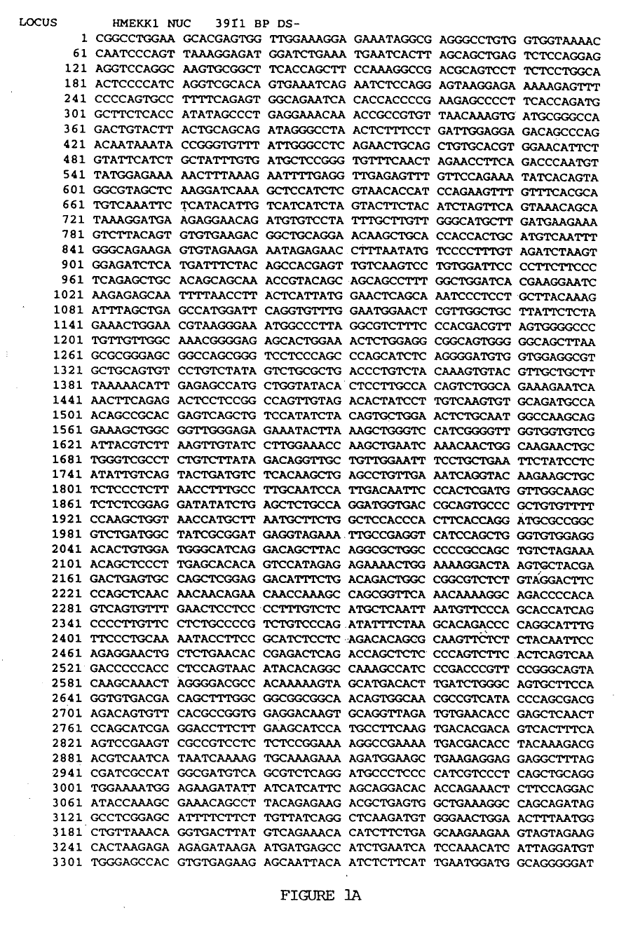

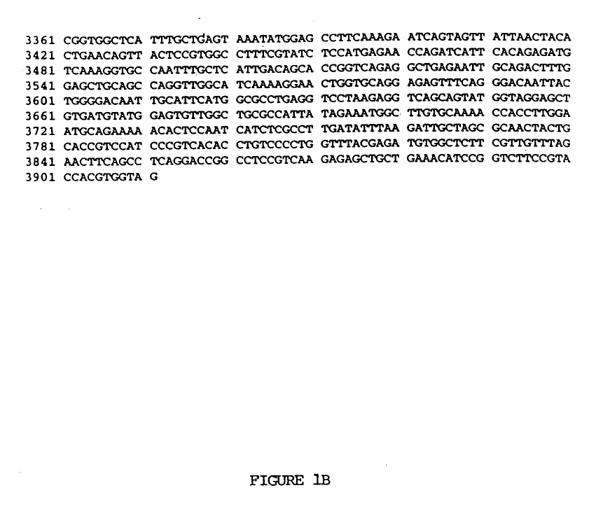

### DETAILED DESCRIPTION OF THE INVENTION

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

The present invention concerns the discovery of novel mitogen ERK kinase kinase proteins (referred to herein as “MEK kinases”, “MEKKs” or “MEKK proteins”) which function in intracellular signal transduction pathways in a variety of cells, and accordingly have a role in determining cell/tissue fate and maintenance. A salient feature of the MEKK1 gene product is the discovery of the involvement of MEKK1 proteins in certain apoptotic pathways.

Accordingly, certain aspects of the present invention relate to nucleic acids encoding vertebrate MEKK1 proteins (e.g., human and murine MEKK1 proteins), the, MEKK1 proteins themselves, antibodies immunoreactive with MEKK1 proteins, and preparations of such compositions. Moreover, the present invention provides diagnostic and therapeutic assays and reagents for detecting and treating disorders involving, for example, aberrant expression or activation of the MEKK1 gene products. In addition, drug discovery assays are provided for identifying agents which can modulate the biological function of MEKK1 proteins, such as by altering the binding of the protein to either downstream or upstream elements in a signal transduction pathway, or which inhibit the kinase activity of the MEKK1 protein. Such agents can be useful therapeutically to alter the growth and/or differentiation of a cell. Other aspects of the invention are described below or will be apparent to those skilled in the art in light of the present disclosure.

One aspect of the present invention relates to isolated MEKK proteins. As used herein protein, peptide, and polypeptide are meant to be synonymous. According to the present invention, an isolated protein is a protein that has been removed from its natural milieu. It will be understood that “isolated”, with respect to MEKK polypeptides, is meant to include formulations of the polypeptides which are isolated from, or otherwise substantially free of other cellular proteins (“contaminating proteins”), especially other cellular signal transduction factors, normally associated with the MEKK polypeptide. Thus, isolated MEKK protein preparations include preparations having less than 20% (by dry weight) contaminating protein, and preferably having less than 5% contaminating protein (but water, buffers, and other small molecules, especially molecules having a molecular weight of less than 5000, can be present). Functional forms of the subject MEKK polypeptides can be prepared, for the first time, as purified preparations by using a cloned gene as described herein. Alternatively, the subject MEKK polypeptides can be isolated by affinity purification using, for example, a catalytically inactive MEK. “Isolated” does not encompass either natural materials in their native state or natural materials that have been separated into components (e.g., in an acrylamide gel) but not obtained either as pure (e.g., lacking contaminating proteins, or chromatography reagents such as denaturing agents and polymers, e.g., acrylamide or agarose) substances or solutions.

An isolated MEKK protein can, for example, be obtained from its natural source, be produced using recombinant DNA technology, or be synthesized chemically. As used herein, an isolated MEKK protein can be a full-length MEKK protein or any homologue of such a protein, such as a MEKK protein in which amino acids have been deleted (e.g., a truncated version of the protein, such as a peptide), inserted, inverted, substituted and/or derivatized (e.g., by glycosylation, phosphorylation, acetylation, myristoylation, prenylation, palmitoylation, amidation and/or addition of glycosylphosphatidyl inositol), wherein the modified protein retains a MEKK biological activity (e.g., is capable of phosphorylating MAP kinase kinases, such as mitogen ERK kinases (MEKs (MKK1 and MKK2)) and/or Jun kinase kinases (JNKKs (MKK3 and MKK4)).

A homologue of a MEKK protein is a protein having an amino acid sequence that is substantially similar or homologous to a natural MEKK protein amino acid sequence that a nucleic acid sequence encoding the homologue is capable of hybridizing under stringent conditions to (i.e., with) a nucleic acid sequence encoding the natural MEKK protein amino acid sequence. As used herein, stringent hybridization conditions refer to standard hybridization conditions under which nucleic acid molecules, including oligonucleotides, are used to identify similar nucleic acid molecules. Such standard conditions are disclosed, for example, in Sambrook et al., Molecular Cloning: A Laboratory Manual, Cold Spring Harbor Labs Press (1989). Exemplary stringent hybridization conditions include but are not limited to hybridization at 65° C. in 4×SSC or at 42° C. in 4×SSC, 50% formamide, followed by washing at 65° C. in 1×SSC. Exemplary high stringency conditions include but are not limited to hybridization at 65° C. in 1×SSC or at 42° C. in 1×SSC, 50% formamide followed by washing at 65° C. in 0.3×sSSC. A homologue of a MEKK protein also includes a protein having an amino acid sequence that is sufficiently cross-reactive such that the homologue has the ability to elicit an immune response against at least one epitope of a naturally-occurring MEKK protein.

The minimal size of a protein homologue of the present invention is a size sufficient to be encoded by a nucleic acid molecule capable of forming a stable hybrid with the complementary sequence of a nucleic acid molecule encoding the corresponding natural protein. As such, the size of the nucleic acid molecule encoding such a protein homologue is dependent on nucleic acid composition, percent homology between the nucleic acid molecule and complementary sequence, as well as upon hybridization conditions per se (e.g., temperature, salt concentration, and formamide concentration). The minimal size of such nucleic acid molecules is typically at least about 12 to about 15 nucleotides in length if the nucleic acid molecules are GC-rich and at least about 15 to about 17 bases in length if they are AT-rich. As such, the minimal size of a nucleic acid molecule used to encode a MEKK protein homologue of the present invention is from about 12 to about 18 nucleotides in length. There is no limit, other than a practical limit, on the maximal size of such a nucleic acid molecule in that the nucleic acid molecule can include a portion of a gene, an entire gene, or multiple genes, or portions thereof. Similarly, the minimal size of a MEKK protein homologue of the present invention is from about 4 to about 6 amino acids in length, with preferred sizes depending on whether a full-length, multivalent protein (i.e., fusion protein having more than one domain each of which has a function), or a functional portion of such a protein is desired.

In another embodiment, a homologue of a MEKK protein is a protein having an amino acid sequence that is at least about 60-65%, 70-75%, 80-85%, 86%, 87%, 88%, 89%, 90%, 91%, 92%, 93%, 94%, 95%, 96%,97%, 98%, or 99% to an amino acid sequence of SEQ ID NO:4 or SEQ ID NO:6 or a portion or fragment thereof. Alternatively, a MEKK homologue is a protein which is encoded by a nucleic acid molecule having at least 60-65%, 70-75%, 80-85%, 86%, 87%, 88%, 89%, 91%, 92%, 93%, 94%, 95%, 96%, 97%, 98%, or 99% homology to a nucleic acid sequence of SEQ ID NO:3 or SEQ ID NO:5. As used herein the term “% homology” can be used interchangeably with the term “% identity”.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989) which has been incorporated into the ALIGN program (version 2.0) (available at http://vega.igh.cnrs.fr/bin/align-guess.cgi), using a PAM120 weight residue table, a gap length penalty of 12 and a gap penalty of 4.

The nucleic acid and protein sequences of the present invention can further be used as a “query sequence” to perform a search against public databases to, for example, identify other family members Nor related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to MSP-18 nucleic acid molecules of the invention. BLAST protein searches can be performed with the XBLAST program, score=50, wordlength=3 to obtain amino acid sequences homologous to MSP-18 protein molecules of the invention. To obtain gapped alignments for comparison purposes, Gapped BLAST can be utilized as described in Altschul et al, (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

MEKK protein homologues can be the result of allelic variation of a natural gene encoding a MEKK protein. A natural gene refers to: the form of the gene found most often in nature. MEKK protein homologues can be produced using techniques known in the art including, but not limited to, direct modifications to a gene encoding a protein using, for example, classic or recombinant DNA techniques to effect random or targeted mutagenesis. As will be understood, mutagenesis includes point mutations, as well as deletions and truncations of the MEKK polypeptide sequence. The ability of a MEKK protein homologue to phosphorylate MEK and/or JNKK protein can be tested using techniques known to those skilled in the art.

With respect to homologues, it will also be possible to modify the structure of the subject MEKK polypeptides forsuch purposes as enhancing therapeutic or prophylactic efficacy, or stability (e.g., ex vivo shelf life and resistance to proteolytic degradation in vivo). Such modified polypeptides, when designed to retain at least one activity of the naturally-occurring form of the protein, are considered functional equivalents of the MEKK polypeptide described in more detail herein. Such modified peptide can be produced, for instance, by amino acid substitution, deletion, or addition.

In one aspect of the invention, the MEKK proteins and/or MEKK homologues are defined as having a MEKK “activity” or “biological activity”. In one embodiment, the MEKK protein is involved in a pathway controlling the phosphorylation of a mitogen-activated protein (MAP) kinase. The mammalian MAP kinase family includes, for example, the extracellular signal-regulated protein kinases (ERK1 and ERK2), p42 or p44 MAPKs. In another preferred embodiment the MEKK protein will be involved in the pathway controlling c-Jun NH2-terminal kinases (JNKs, or SAPKs), and the so-called “p38 subgroup” kinases (p38 and Hog-1 kinases). For example, it is contemplated that the MEKK proteins of the present invention interact with, and directly phosphorylate members of the MAP kinase kinase family (MEKs or MKKs), as MEK1, MEK2, MKK1, MKK2, or the stress-activated kinases (SEKs), and the Jun kinase kinases (JNKK1, JNKK2, MKK3, MKK4), or the like. An exemplary MEKK-dependent pathway includes a pathway involving a MEKK protein and a MKK protein.

In another embodiment, a MEKK protein is capable of regulating the activity of signal transduction proteins including, but not limited to, mitogen activated ERK kinases (MEKs), mitogen activated protein kinases (MAPKs), transcription control factor (TCF), Ets-like-1 transcription factor (Elk-1), Jun ERK kinases (JNKKs), Jun kinases (JNK; which is equivalent to SAPK), stress activated MAPK proteins, Jun, activating transcription factor-2 (ATF-2) and/or Myc protein.

As used herein, the “activity” or “biological activity” of a protein can be directly correlated with the phosphorylation state of the protein and/or the ability of the protein to perform a particular function (e.g., phosphorylate another protein or regulate transcription). Preferred MEK proteins regulated by a MEKK protein of the present invention include MEK-1 and/or MEK-2 (MKK1 or MKK2). Preferred MAPK proteins regulated by a MEKK protein of the present invention include p38/Hog-1 MAPK, p42 MAPK and/or p44 MAPK. Preferred stress activated MAPK proteins regulated by a MEKK protein of the present invention include Jun kinase (JNK), stress activated MAPK-α and/or stress activated MAPK-β.

In a preferred embodiment, a MEKK protein of the present invention is capable of phosphorylating a MEK or MKK, Jun kinase kinase (JNKK) and/or stress activated ERK kinase (SEK), in particular MEK1, MEK2, MKK1, MKK2, MKK3, MKK4, JNKK1, JNKK2, SEK1 and/or SEK2 proteins. As described herein, MKK1 and MEK2 are equivalent to MKK1 and MKK2, respectively. In addition, JNKK1 and JNKK2, are equivalent to MKK3 and MKK4, which are equivalent to SEK1 and SEK2.

A preferred MEKK protein of the present invention is additionally capable of inducing Myc proteins and members of the Ets family of transcription factors, such as TCF protein, also referred to as Elk-1 protein.

Another aspect of the present invention is the recognition that a MEKK protein of the present invention is capable of regulating the apoptosis of a cell As used herein, apoptosis refers to the form of cell death that comprises: progressive contraction of cell volume with the preservation of the integrity of cytoplasmic organelles; condensation of chromatin, as viewed by light or electron microscopy; and DNA cleavage, as electrophoresis or labeling of DNA fragments using terminal deoxytransferase (TDT). Cell death occurs when the membrane integrity of the cell is lost and cell lysis occurs. Apoptosis differs from necrosis in which cells swell and eventually rupture.

A preferred MEKK protein of the present invention is capable of inducing the apoptosis of cells, such that the cells have characteristics substantially similar to cytoplasmic shrinkage and/or nuclear condensation.

A schematic representation of exemplary cell growth regulatory pathways that are MEKK dependent is shown in FIG. 6.

In addition to the numerous functional characteristics of a MEKK protein, a MEKK protein of the present invention comprises numerous unique structural characteristics. For example, in one embodiment, a MEKK protein of the present invention includes at least one of two different structural domains having particular functional characteristics. Such structural domains include an NH₂-terminal regulatory domain that serves to regulate a second structural domain comprising a COOH-terminal protein kinase catalytic domain that is capable of phosphorylating an MKK protein.

According to the present invention, a MEKK protein of the present invention includes a full-length MEKK protein, as well as at least a portion of a MEKK protein capable of performing at least one of the functions defined above. Preferred portions are capable of inducing apoptosis. The phrase “at least a portion of a MEKK protein”, refers to a portion of a MEKK protein encoded by a nucleic acid molecule that is capable of hybridizing, under stringent conditions, with a nucleic acid encoding a full-length MEKK protein of the present invention. Preferred portions of MEKK proteins are useful for regulating apoptosis in a cell. Suitable sizes for portions of a MEKK protein of the present invention are 65-70 kD, 75-80 kD, 85-90 kD, 100-110 kD, 120-130 kD, 140-150 kD, 160-170 kD or 180 kD or larger as determined by Tris-glycine SDS-PAGE, preferably using an 8% polyacrylamide SDS gel (SDS-PAGE) and resolved using methods standard in the art. It is noted that experimental conditions used when running gels to determine the molecular size of putative MEKK proteins and/or portions thereof will cause variations in results.

In one embodiment, a portion of a MEKK protein capable of inducing apoptosis includes about amino acids 875-1493 of murine MEKK1, set forth in SEQ ID NO:4. In another embodiment, a portion of a MEKK1 protein capable of inducing apoptosis includes about amino acids 685-1303 of human MEKK1, set forth in SEQ ID NO:6. In another embodiment, a portion of a MEKK protein capable of inducing apoptosis is substantially similar or homologous to amino acids 875-1493 of murine MEKK1, set forth in SEQ ID NO:4. In another embodiment, a portion of a MEKK protein capable of inducing apoptosis is substantially similar or homologous to amino acids 685-1303 of human MEKK1, set forth in SEQ ID NO:6.

The sequences comprising the catalytic domain of a MEKK protein are involved in phosphotransferase activity, and therefore display a relatively conserved amino acid sequence. The NH₂-terminal regulatory domain of a MEKK protein, however, can be substantially divergent. As such, the NH₂-terminal regulatory domain of a MEKK protein provides selectivity for upstream signal transduction regulation, while the catalytic domain provides for MEKK substrate selectivity function.

In another embodiment, the subject MEKK proteins are provided as fusion proteins. It is widely appreciated that fusion proteins can also facilitate the expression of proteins, and accordingly, can be used in the expression of the MEKK polypeptides of the present invention. For example, MEKK polypeptides can be generated as glutathione-S-transferase (GST-fusion) proteins. Such GST-fusion proteins can enable easy purification of the MEKK polypeptide, as for example by the use of glutathione-derivatized matrices (see, for example, Current Protocols in Molecular Biology, eds. Ausubel et al. (N.Y.: John Wiley & Sons, 1991)).

In another embodiment, a fusion gene coding for a purification leader sequence, such as a poly-(His)/enterokinase cleavage site sequence at the N-terminus of the desired portion of the recombinant protein, can allow purification of the expressed fusion protein by affinity chromatography using a Ni2+ metal resin. The purification leader sequence can then be subsequently removed by treatment with enterokinase to provide the purified protein (e.g., see Hochuli et al. (1987) J. Chromatography 411:177; and Janknecht et al. PNAS 88:8972).

Techniques for making fusion genes are known to those skilled in the art. Essentially, the joining of various DNA fragments coding for different polypeptide sequences is performed in accordance with conventional techniques, employing blunt ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al. John Wiley & Sons: 1992).

According to the present invention, a MEKK protein of the present invention can include MEKK proteins that have undergone post-translational modification. Such modification can include, for example, phosphorylation or among other post-translational modifications including conformational changes or post-translational deletions.

This invention further contemplates a method for generating sets of combinatorial mutants of the subject MEKK proteins as well as truncation mutants, and is especially useful for identifying potential variant sequences (e.g., homologs) that are functional in modulating signal transduction. The purpose of screening such combinatorial libraries is to generate, for example, novel MEKK homologs which can act as either agonists or antagonist of the wild-type MEKK proteins, or alternatively, which possess novel activities all together. To illustrate, MEKK homologs can be engineered by the present method to provide selective, constitutive activation of a pathway, so as mimic induction by a factor when the MEKK homolog is expressed in a cell capable of responding to the factor. Thus, combinatorially-derived homologs can be generated to have an increased potency relative to a naturally occurring form of the protein.

Likewise, MEKK homologs can be generated by the present combinatorial approach to selectively inhibit (antagonize) induction by a growth or other factor. For instance, mutagenesis can provide MEKK homologs which are able to bind other signal pathway proteins (e.g., MEKs) yet prevent propagation of the signal, e.g., the homologs can be dominant negative mutants. Moreover, manipulation of certain domains of MEKK by the present method can provide domains more suitable for use in fusion proteins.

In one aspect of this method, the amino acid sequences for a population of MEKK homologs or other related proteins are aligned, preferably to promote the highest homology possible. Such a population of variants can include, for example, MEKK homologs from one or more species. Amino acids which appear at each position of the aligned sequences are selected to create a degenerate set of combinatorial sequences. In a preferred embodiment, the variegated library of MEKK variants is generated by combinatorial mutagenesis at the nucleic acid level, and is encoded by a variegated gene library. For instance, a mixture of synthetic oligonucleotides can be enzymatically ligated into gene sequences such that the degenerate set of potential MEKK sequences; are expressible as individual polypeptides, or alternatively, as a set of larger fusion proteins (e.g., for phage display) containing the set of MEKK sequences therein.

There are many ways by which such libraries of potential MEKK homologs can be generated from a degenerate oligonucleotide sequence. Chemical synthesis of a degenerate gene sequence can be carried out in an automatic DNA synthesizer, and the synthetic genes then ligated into an appropriate expression vector. The purpose of a degenerate set of genes is to provide, in one mixture, all of the sequences encoding the desired set of potential MEKK sequences. The synthesis of degenerate oligonucleotides is well known in the art (see for example, Narang, S A (1983), Tetrahedron 39:3; Itakura et al. (1981) Recombinant DNA, Proc 3rd Cleveland Sympos. Macromolecules, ed. AG Walton, Amsterdam: Elsevier pp273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

Likewise, a library of coding sequence fragments can be provided for a MEKK clone in order to generate a variegated population of MEKK fragments for screening and subsequent selection of bioactive fragments. A variety of techniques are known in the art for generating such libraries, including chemical synthesis. In one embodiment, a library of coding sequence fragments can be generated by (i) treating a double stranded PCR fragment of a MEKK coding sequence with a nuclease under conditions wherein nicking occurs only about once per molecule; (ii) denaturing the double stranded DNA; (iii) renaturing the DNA to form double stranded DNA which can include sense/antisense pairs from different nicked products; (iv) removing single stranded portions from reformed duplexes by treatment with S1 nuclease; and (v) ligating the resulting fragment library into an expression vector. By this exemplary method, an expression library can be derived which codes for N-terminal, C-terminal and internal fragments of various sizes.

A wide range of techniques are known in the art for screening gene products of combinatorial libraries made by point mutations or truncation, and for screening cDNA libraries for gene products having a certain property. Such techniques will be generally adaptable for rapid screening of the gene libraries generated by the combinatorial mutagenesis of MEKK homologs. The most widely used techniques for screening large gene libraries typically comprise cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate MEKK sequences created by combinatorial mutagenesis techniques.

In an illustrative embodiment of a screening assay, the gene library can be expressed as a fusion protein on the surface of a viral particle. For instance, in the filamentous phage system, foreign peptide sequences can be expressed on the surface of infectious phage, thereby conferring two significant benefits. First, since these phage can be applied to affinity matrices at very high concentrations, a large number of phage can be screened at one time. Second, since each infectious phage displays the combinatorial gene product on its surface, if a particular phage is recovered from an affinity matrix in low yield, the phage can be amplified by another round of infection. The group of almost identical E. coli filamentous phages M13, fd, and f1 are most often used in phage display libraries, as either of the phage gIII or gVIII coat proteins can be used to generate fusion proteins without disrupting the ultimate packaging of the viral particle (Ladner et al. PCT publication WO 90/02909; Garrard et al., PCT publication WO 92/09690; Marks et al. (1992) J. Biol. Chem. 267:16007-16010; Griffths et al. (1993) EMBO J 12:725-734; Clackson et al. (1991) Nature 352:624-628; and Barbas et al. (1992)PNAS 89:4457-4461). The resulting phage libraries with the fusion tail proteins may be panned, e.g., using a fluorescent labeled MEK protein, e.g., FITC-MEK, to score for MEKK homologs which retain the ability to bind to the MEK protein. Individual phage which encode a MEKK homolog which retains MEK binding can be isolated, the MEKK homolog gene recovered from the isolate, and further tested to discern between active and antagonistic mutants

In another embodiment, cells (e.g., REF52 cells) can be exploited to analyze the variegated MEKK library. For instance, the library of expression vectors can be transfected into a population of REF52 cells which also inducibly overexpress a MEKK protein (e.g., and which overexpression causes apoptosis). Expression of WT-MEKK is then induced, and the effect of the MEKK mutant on induction of apoptosis can be detected. Plasmid DNA can then be recovered from the cells which score for inhibition, or alternatively, potentiation of apoptosis, and the individual clones further characterized.

The invention also provides for reduction of the MEKK proteins to generate mimetics, e.g., peptide or non-peptide agents, which are able to disrupt binding of a MEKK polypeptide of the present invention with either upstream or downstream components of its signaling cascade. Thus, such mutagenic techniques as described above are also useful to map the determinants of the MEKK proteins which participate in protein-protein interactions involved in, for example, binding of the subject MEKK polypeptide to proteins which may function upstream (including both activators and repressors of its activity) or to proteins which may function downstream of the MEKK polypeptide, whether they are positively or negatively regulated by it. To illustrate, the critical residues of a subject MEKK polypeptide which are involved in molecular recognition of an upstream or downstream MEKK component can be determined and used to generate MEKK-derived peptidomimetics which competitively inhibit binding of the authentic protein with that moiety. By employing, for example, scanning mutagenesis to map the amino acid residues of each of the subject MEKK proteins which are involved in binding other cellular proteins, peptidomimetic compounds can be generated which mimic those residues of the MEKK protein which facilitate the interaction. Such mimetics may then be used to interfere with the normal function of a MEKK protein. For instance, non-hydrolyzable peptide analogs of such residues can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), and β-aminoalcohols (Gordon et al. (1985) Biochem Biophys Res Commun 126:419; and Dann et al. (1986) Biochem Biophys Res Commun 134:71).

Another aspect of the present invention is an isolated nucleic acid molecule capable of hybridizing, under stringent conditions, with a MEKK protein gene encoding a MEKK protein of the present invention. In accordance with the present invention, an isolated nucleic acid molecule is a nucleic acid molecule that has been removed from its natural milieu (i.e., that has been subject to human manipulation). As such, “isolated” does not reflect the extent to which the nucleic acid molecule has been purified. To this end, the term “isolated” as used herein with respect to nucleic acids, such as DNA or RNA, refers to molecules separated from other DNAs, or RNAs, respectively, that are present in the natural source of the macromolecule. For example, an isolated nucleic acid encoding one of the subject MEKK polypeptides preferably includes no more than 10 kilobases (kb) of nucleic acid sequence which naturally immediately flanks the MEKK gene in genomic DNA, more preferably no more than 5 kb of such naturally occurring flanking sequences, and most preferably less than 1.5 kb of such naturally occurring flanking sequence. In another example, the isolated MEKK nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. The term isolated as used herein will also be understood to include nucleic acid that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state.

An isolated nucleic acid molecule can include DNA, RNA, or derivatives of either DNA or RNA. Accordingly, as used herein, the term “nucleic acid” includes polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

As used herein, the term “gene” or “recombinant gene” includes nucleic acid comprising an open reading frame encoding one of the MEKK polypeptides of the present invention, including both exon and (optionally) intron sequences. A “recombinant gene” refers to nucleic acid encoding a MEKK polypeptide (e.g., a vertebrate MEKK polypeptide) and comprising MEKK-encoding exon sequences, though it may optionally include intron sequences which are either derived from a chromosomal MEKK gene or from an unrelated chromosomal gene. Exemplary recombinant genes encoding the subject MEKK polypeptides are represented in the appended Sequence Listing. The term “intron” refers to a DNA sequence present in a given MEKK gene which is not translated into protein and is generally found between exons.

An isolated nucleic acid molecule of the present invention can be obtained from its natural source either as an entire (i.e., complete) gene or a portion thereof capable of forming a stable hybrid with that gene. As used herein, the phrase “at least a portion of” an entity refers to an amount of the entity that is at least sufficient to have the functional aspects of that entity. For example, at least a portion of a nucleic acid sequence, as used herein, is an amount of a nucleic acid sequence capable of forming a stable hybrid with a particular desired gene (e.g., MEKK genes) under stringent hybridization conditions. An isolated nucleic acid molecule of the present invention can also be produced using recombinant DNA technology (e.g., polymerase chain reaction (PCR) amplification, cloning) or chemical synthesis. Isolated MEKK protein nucleic acid molecules include natural nucleic acid, molecules and homologues thereof, including, but not limited to, natural allelic variants and modified nucleic acid molecules in which nucleotides have been inserted, deleted, substituted, and/or inverted in such a manner that such modifications do not substantially interfere with the nucleic acid molecule's ability to encode a MEKK protein of the present invention or to form stable hybrids under , stringent conditions with natural nucleic acid molecule isolates of MEKK.

Preferred modifications to a MEKK protein nucleic acid molecule of the present invention include truncating a full-length MEKK protein nucleic acid molecule by, for example: deleting at least a portion of a MEKK protein nucleic acid molecule encoding a regulatory domain to produce a constitutively active MEKK protein; deleting at least a portion of a MEKK protein nucleic acid molecule encoding a catalytic domain to produce an inactive MEKK protein; and modifying the MEKK protein to achieve desired inactivation and/or stimulation of the protein, for example, substituting a codon encoding a lysine residue in the catalytic domain (i.e., phosphotransferase domain) with a methionine residue to inactivate the catalytic domain.

A preferred truncated MEKK nucleic acid molecule encodes a form of a MEKK protein which is capable of inducing apoptosis.

An isolated nucleic acid molecule of the present invention can include a nucleic acid sequence that encodes at least one MEKK protein of the present invention, examples of such proteins being disclosed herein. Although the phrase “nucleic acid molecule” primarily refers to the physical nucleic acid molecule and the phrase “nucleic acid sequence” primarily refers to the sequence of nucleotides that comprise the nucleic acid molecule, the two phrases can be used interchangeably. As heretofore disclosed, MEKK proteins of the present invention include, but are not limited to, proteins having full-length MEKK protein coding regions, portions thereof, and other MEKK protein homologues.

As used herein, a MEKK protein gene includes all nucleic acid sequences related to a natural MEKK protein gene such as regulatory regions that control production of a MEKK protein encoded by that gene (including, but not limited to, transcription, translation or post-translation control regions) as well as the coding region itself. A nucleic acid molecule of the present invention can be an isolated natural MEKK protein nucleic acid molecule or a homologue thereof. A nucleic acid molecule of the present invention can include one or more regulatory regions, full-length or partial coding regions, or combinations thereof. The minimal size of a MEKK protein nucleic acid molecule of the present invention is the minimal size capable of forming a stable hybrid under stringent hybridization conditions with a corresponding natural gene.

A MEKK protein nucleic acid molecule homologue can be produced using a number, of methods known to those skilled in the art (see, e.g., Sambrook et al., ibid.). For example, nucleic acid molecules can be modified using a variety of techniques including, but not limited to, classic mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid molecules and combinations thereof. Nucleic acid molecule homologues can be selected from a mixture of modified nucleic acids by screening for the function of the protein encoded by the nucleic acid (e.g., the ability of a homologue to phosphorylate MEK protein or JNKK protein) and/or by hybridization with isolated MEKK protein nucleic acids under stringent conditions.

A preferred nucleic acid molecule of the present invention is capable of hybridizing under stringent conditions to a nucleic acid that encodes at least a portion of a MEKK protein, or a homologue thereof. Also preferred is a MEKK nucleic acid molecule that includes a nucleic acid sequence having at least about 50% homology, preferably 75% homology, preferably 85% homology, or even more preferably 95% homology with an MEKK nucleic acid molecule of the invention. In other embodiments nucleic acids have 50%, preferably at least about 75%, and more preferably at least about 85%, and most preferably at least about 95% homology with the corresponding region(s) of the nucleic acid sequence encoding the catalytic domain of a MEKK protein, or a homologue thereof. Also preferred is a MEKK protein nucleic acid molecule that includes a nucleic acid sequence having at least about 50%, preferably at least about 75%, more preferably at least about 85%, and even more preferably at least about 95% homology with the corresponding region(s) of the nucleic acid sequence encoding the NH2-terminal regulatory domain of a MEKK protein, or a homologue thereof. Such nucleic acid molecules can be a full-length gene and/or a nucleic acid molecule encoding a full-length protein, a hybrid protein, a fusion-protein, a multivalent protein or a truncation fragment.

Knowing a nucleic acid molecule of a MEKK protein of the present invention allows one skilled in the art to make copies of that nucleic acid molecule as well as to obtain additional portions of MEKK protein-encoding genes (e.g., nucleic acid molecules that include the translation start site and/or transcription and/or translation control regions), and/or MEKK protein nucleic acid molecule homologues. Knowing a portion of an amino acid sequence of a MEKK protein of the present invention allows one skilled in the art to clone nucleic acid sequences encoding such a MEKK protein.

The present invention also includes nucleic acid molecules that are oligonucleotides capable of hybridizing, under stringent conditions, with complementary regions of other, preferably longer, nucleic acid molecules of the present invention that encode at least a portion of a MEKK protein, or a homologue thereof. A preferred oligonucleotide is capable of hybridizing, under stringent conditions, with a nucleic acid molecule of SEQ ID NO: 3 or SEQ ID NO:5.

Oligonucleotides of the present invention can be RNA, DNA, or derivatives of either. The minimal size of such oligonucleotides is the size required to form a stable hybrid between a given oligonucleotide and the complementary sequence on another nucleic acid molecule of the present invention. Minimal size characteristics of preferred oligonucleotides are at least about 10 nucleotides, preferably at least about 20 nucleotides, more preferably at least about 50 nucleotides and most preferably at least about 60 nucleotides. Larger fragments are also contemplated. The size of the oligonucleotide must also be sufficient for the use of the oligonucleotide in accordance with the present invention. Oligonucleotides of the present invention can be used in a variety of applications including, but not limited to, as probes to identify additional nucleic acid molecules, as primers to amplify or extend nucleic acid molecules or in therapeutic applications to inhibit, for example, expression of MEKK proteins by cells. Such therapeutic applications include the use of such oligonucleotides in, for example, antisense-, triplex formation-, ribozyme- and/or RNA drug-based technologies. The present invention, therefore, includes use of such oligonucleotides and methods to interfere with the production of MEKK proteins. In addition oligonucleotides encoding portions of MEKK proteins which bind to MEKK binding proteins can be used a therapeutics. In other embodiments, the peptides encoded by these nucleic acids are used.

To further illustrate, another aspect of the invention relates to the use of the isolated nucleic acid in “antisense” therapy. As used herein, “antisense” therapy refers to administration or in situ generation of oligonucleotide probes or their derivatives which specifically hybridize (e.g., bind) under cellular conditions, with the cellular mRNA and/or genomic DNA encoding one or more of the subject MEKK proteins so as to inhibit expression of that protein, e.g., by inhibiting transcription and/or translation. The binding may be by conventional base pair complementarity, or, for example, in the case of binding to DNA duplexes, through specific interactions in the major groove of the double helix. In general, “antisense” therapy refers to the range of techniques generally employed in the art, and includes any therapy which relies on specific binding to oligonucleotide sequences.

An antisense construct of the present invention can be delivered, for example, as an expression plasmid which, when transcribed in the cell, produces RNA which is complementary to at least a unique portion of the cellular mRNA which encodes a vertebrate MEKK protein. Alternatively, the antisense construct is an oligonucleotide probe which is generated ex vivo and which, when introduced into the cell causes inhibition of expression by hybridizing with the mRNA and/or genomic sequences of a vertebrate MEKK gene. Such oligonucleotide probes are preferably modified oligonucleotides which are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed; for example, by Van der Krol et al. (1988) Biotechniques 6:958-976; and Stein et al. (1988) Cancer Res 48:2659-2668.

Accordingly, the modified oligomers of the invention are useful in therapeutic, diagnostic, and research contexts. In therapeutic applications, the oligomers are utilized in a manner appropriate for antisense therapy in general. For such therapy, the oligomers of the invention can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the oligomers of the invention can be formulated in liquid solutions, preferably in physiologically, compatible buffers such as Hank's solution or Ringer's solution. In addition, the oligomers may be formulated in solid form and redissolved or suspended immediately prior to use. Lyophilized forms are also included.

Systemic administration can also be by transmucosal or transdermal means, or the compounds can be administered orally. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For oral administration, the oligomers are formulated into conventional oral administration forms such as capsules, tablets, and tonics. For topical administration, the oligomers of the invention are formulated into ointments, salves, gels, or creams as generally known in the art.

In addition to use in therapy, the oligomers of the invention may be used as diagnostic reagents to detect the presence or absence of the target DNA or RNA sequences to which they specifically bind. Such diagnostic tests are described in further detail below.

Likewise, the antisense constructs of the present invention, by antagonizing the normal biological activity of one of the MEKK proteins, can be used in the manipulation of tissue, e.g., tissue differentiation, both in vivo and for ex vivo tissue cultures.

Furthermore, the anti-sense techniques (e.g., microinjection of antisense molecules, or transfection with plasmids whose transcripts are anti-sense with regard to a MEKK mRNA or gene sequence) can be used to investigate role of MEKK in disease states, as well as the normal cellular function of MEKK in healthy tissue. Such techniques can be utilized in: cell culture, but can also be used in the creation of transgenic animals. The present invention also includes a recombinant vector which includes at least one MEKK protein nucleic acid molecule of the present invention inserted into any vector capable of delivering the nucleic acid molecule into a host cell. Such a vector contains heterologous nucleic acid sequences, for example nucleic acid sequences that are not naturally found adjacent to MEKK protein nucleic acid molecules of the present invention. The vector can be either RNA or DNA, and either prokaryotic or eukaryotic, and is typically a virus or a plasmid. Recombinant vectors can be used in the cloning, sequencing, and/or otherwise manipulating of MEKK protein nucleic acid molecules of the present invention. One type of recombinant vector, herein referred to as a recombinant molecule and described in more detail below, can be used in the expression of nucleic acid molecules of the present invention. Preferred recombinant vectors are capable of replicating in the transformed cell.

Preferred nucleic acid molecules to insert into a recombinant vector includes a nucleic acid molecule that encodes at least a portion of a MEKK protein, or a homologue thereof. In particularly preferred embodiments portions of a MEKK nucleic acid which encodes a MEKK catalytic domain is used. In another particularly preferred embodiment, at least a portion of a nucleic acid which encodes the portion of a MEKK protein which binds to a MEKK substrate or a MEKK regulatory protein is used.

Preferred nucleic acid molecules for insertion into an expression vector include nucleic acid molecules that encode at least a portion of a MEKK protein, or a homologue thereof.

Expression vectors of the present invention may also contain fusion sequences which lead to the expression of inserted nucleic acid molecules of the present invention as fusion proteins. Inclusion of a fusion sequence as part of a MEKK nucleic acid molecule of the present invention can enhance the stability during production, storage and/or use of the protein encoded by the nucleic acid molecule. Furthermore, a fusion segment can function as a tool to simplify purification of a MEKK protein, such as to enable purification of the resultant fusion protein using affinity chromatography. A suitable fusion segment can be a domain of any size that has the desired function (e.g., increased stability and/or purification tool). It is within the scope of the present invention to use one or more fusion segments. Fusion segments can be joined to amino and/or carboxyl termini of a MEKK protein. Linkages between fusion segments and MEKK proteins can be constructed to be susceptible to cleavage to enable straight-forward recovery of the MEKK proteins. Fusion proteins are preferably produced by culturing a recombinant cell transformed with a fusion nucleic acid sequence that encodes a protein including the fusion segment attached to either the carboxyl and/or amino terminal end of a MEKK protein.

Moreover, the gene constructs of the present invention can also be used as a part of a gene therapy protocol to deliver nucleic acids encoding either an agonistic or antagonistic form of one of the subject MEKK proteins. Thus, another aspect of the invention features expression vectors for in vivo or in vitro transfection and expression of a MEKK polypeptide in particular cell types so as to reconstitute the function of, constitutively activate, or alternatively, abrogate the function of a signal pathway dependent on a MEKK activity. Such therapies may useful where the naturally-occurring form of the protein is misexpressed or inappropriately activated; or to deliver a form of the protein which alters differentiation of tissue; or which inhibits neoplastic transformation.

Expression constructs of the subject MEKK polypeptide, and mutants thereof, may be administered in any biologically effective carrier, e.g., any formulation or composition capable of effectively delivering the recombinant gene to cells in vivo. Approaches include insertion of the subject gene in viral vectors including recombinant retroviruses, adenovirus, adeno-associated virus, and herpes simplex virus-1, or recombinant bacterial or eukaryotic plasmids. Viral vectors transfect cells directly; plasmid DNA can be delivered with the help of, for example, cationic liposomes (lipofectin) or derivatized (e.g., antibody conjugated), polylysine conjugates, gramacidin S, artificial viral envelopes or other such intracellular carriers, as well as direct injection of the gene construct or CaPO₄ precipitation carried out in vivo. It will be appreciated that because transduction of appropriate target cells represents the critical first step in gene therapy, choice of the particular gene delivery system will depend on such factors as the phenotype of the intended target and the route of administration, e.g., locally or systemically. Furthermore, it will be recognized that the particular gene construct provided for in vivo transduction of MEKK expression are also useful for in vitro transduction of cells, such as for use in the ex vivo tissue culture systems described below.

A preferred approach for in vivo introduction of nucleic acid into a cell is by use of a viral vector containing nucleic acid, e.g., a cDNA, encoding the particular MEKK polypeptide desired. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors are generally understood to be the recombinant gene delivery system of choice for the transfer of exogenous genes in vivo, particularly into humans. These vectors provide efficient delivery of genes into cells, and the transferred nucleic acids are stably integrated into the chromosomal DNA of the host. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) has been replaced by nucleic acid encoding one of the subject proteins rendering the retrovirus replication defective.: The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines for preparing both ecotropic and amphotropic retroviral systems include ψCrip, ψCre, ψ2 and ψAm. Retroviruses have been used to introduce a variety of genes into many different cell types, including neuronal cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Furthermore, it has been shown that it is possible to limit the infection spectrum of retroviruses and consequently of retroviral-based vectors, by modifying the viral packaging proteins on the surface of the viral particle (see, for example PCT publications WO 93/25234 and WO 94/06920). For instance, strategies for the modification of the infection spectrum of retroviral vectors include: coupling antibodies specific for cell surface antigens to the viral env protein (Roux et al. (1989) PNAS 86:9079-9083; Julan et al. (1992) J. Gen Virol 73:3251-3255; and Goud et al. (1983) Virology 163:251-254); or coupling cell surface receptor ligands to the viral env proteins (Neda et al. (1991) J. Biol Chem 266:14143-14146). Coupling can be in the form of the chemical cross-linking with a protein or other variety (e.g., lactose to convert the env protein to an asialoglycoprotein), as well as by generating fusion proteins (e.g., single-chain antibody/env fusion proteins). This technique, while useful to limit or otherwise direct the infection to certain tissue types, can also be used to convert an ecotropic vector in to an amphotropic vector.

Moreover, use of retroviral gene delivery can be further enhanced by the use of tissue- or cell-specific transcriptional regulatory sequences which control expression of the MEKK gene of the retroviral vector.

Another viral gene delivery system useful in the present invention utilizes adenovirus-derived vectors. The genome of an adenovirus can be manipulated such that it encodes and expresses a gene product of interest but is inactivated in terms of its ability to replicate in a normal lytic viral life cycle. See for example Berkner et al. (1988) Biotechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are well known to those skilled in the art. Recombinant adenoviruses can be advantageous in certain circumstances in that they can be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad. Sci. USA 90:2812-2816) and muscle cells (Quantin et al. (1992) Proc. Natl. Acad. Sci. USA 89:2581-2584). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use and therefore favored by the present invention are deleted for all or parts of the viral E1 and E3 genes but retain as much as 80% of the adenoviral genetic material (see, e.g., Jones et al. (1979) Cell 16:683; Berkner et al., supra; and Graham et al. in Methods in Molecular Biology, E. J. Murray, Ed. (Humana, Clifton, N.J., 1991) vol. 7. pp. 109-127). Expression of the inserted MEKK gene can be under control of, for example, the E1A. promoter, the major late promoter (MLP) and associated leader sequences, the E3 promoter, or exogenously added promoter sequences.

Yet another viral vector system useful for delivery of one of the subject MEKK genes is the adeno-associated virus (AMINO ACIDSV). Adeno-associated virus is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992)158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1 989) J. Virol. 63:3822-3828; and McLaughlin et al. (1989) J. Virol. 62:1963-1973). Vectors containing as little as 300 base pairs of AMINO ACIDSV can be packaged and can integrate. Space for exogenous DNA is limited to about 4.5 kb. An AMINO ACIDSV vector such as that described in Tratschin et al. (1985) Mol. Cell. Biol. 5:3251-3260 can be used to introduce DNA into cells. A variety of nucleic acids have been introduced into different cell types using AMINO ACIDSV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

In addition to viral transfer methods, such as those illustrated above, non-viral methods can also be employed to cause expression of a subject MEKK polypeptide in the tissue of an animal. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the subject MEKK polypeptide gene by the targeted cell. Exemplary gene delivery systems of this type include liposomal derived systems, poly-lysine conjugates, and artificial viral envelopes.

In clinical settings, the gene delivery systems for the therapeutic MEKK gene can be introduced into a patient by any of a number of methods, each of which is familiar in the art. For instance, a pharmaceutical preparation of the gene delivery system can be introduced systemically, e.g., by intravenous injection, and specific transduction of the protein in the target cells occurs predominantly from specificity of transfection provided by the gene delivery vehicle, cell-type or tissue-type expression due to, the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g., Chen et al. (1994) PNAS 91: 3054-3057). A MEKK gene, such as any one of the clones represented in the appended Sequence Listing, can be delivered in a gene therapy construct by electroporation using techniques described, for example, by Dev et al. ((1994) Cancer Treat Rev 20:105-115).

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced intact from recombinant cells, e.g., retroviral vectors, .the pharmaceutical preparation can comprise one or more cells which produce the gene delivery system.

Still another aspect of the present invention pertains to recombinant cells, e.g., cells which are transformed with at least one of any nucleic acid molecule of the present invention. A preferred recombinant cell is a cell transformed with at least one nucleic acid molecule that encodes at least a portion of a MEKK protein, or a homologue thereof.

Suitable host cells for transforming a cell can include any cell capable of producing MEKK proteins of the present invention after being transformed with at least one nucleic acid molecule of the present invention. Host cells can be either untransformed cells or cells that are already transformed with at least one nucleic acid molecule. Suitable host cells of the present invention can include bacterial, fungal (including yeast), insect, animal and plant cells. Preferred host cells include bacterial, yeast, insect and mammalian cells, with mammalian cells being particularly preferred.

A recombinant cell is preferably produced by transforming a host cell with one or more recombinant molecules, each comprising one or more nucleic acid molecules of the present invention operatively linked to an expression vector containing one or more transcription control sequences. The phrase operatively linked refers to insertion of a nucleic acid molecule into an expression vector in a manner such that the molecule is able to be expressed when transformed into a host cell. As used herein, an expression vector is a DNA or RNA vector that is capable of transforming a host cell and of effecting expression of a specified nucleic acid molecule. Preferably, the expression vector is also capable of replicating within the host cell. Expression vectors can be either prokaryotic or eukaryotic, and are typically viruses or plasmids. Expression vectors of the present invention include any vectors that function (i.e., direct gene expression) in recombinant cells of the present invention, including in bacterial, fungal, insect, animal, and/or plant cells.! As such, nucleic acid molecules of the present invention can be operatively linked to expression vectors containing regulatory sequences such as promoters, operators, repressors, enhancers, termination sequences, origins of replication, and other regulatory sequences that are compatible with the recombinant cell and that control the expression of nucleic acid molecules of the present invention. As used herein, a transcription control sequence includes a sequence which is capable of controlling the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in bacterial, yeast, and mammalian cells, such as, but not limited to, tac, lac; trp, trc, oxy-pro, omp/lpp, rrnB, bacteriophage lambda (λ) (such as λp_{L} and λp_{R} and fusions that include such promoters), bacteriophage T7, T7lac, bacteriophage T3, bacteriophage SP6, bactenrophage SP01, metallothionein, alpha mating factor, baculovirus, vaccinia virus, herpesvirus, poxvirus, adenovirus, simian virus 40, retrovirus actin, retroviral long terminal repeat, Rous sarcoma virus, heat shock, phosphate and nitrate transcription control sequences, as well as other sequences capable of controlling gene expression in prokaryotic or eukaryotic cells. Additional suitable transcription control sequences include tissue-specific promoters and enhancers as well as lymphokine-inducible promoters (e.g., promoters inducible by interferons or interleukins). Transcription control sequences of the present invention can also include naturally occurring transcription control sequences naturally associated with a DNA sequence encoding a MEKK protein.

It may be appreciated by one skilled in the art that use of recombinant DNA technologies can improve expression of transformed nucleic acid molecules by manipulating, for example, the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules of the present invention include, but are not limited to, operatively linking nucleic acid molecules to high-copy number plasmids, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgamo sequences), modification of nucleic acid molecules of the present invention to correspond to the codon usage of the host cell, deletion of sequences that destabilize transcripts, and use of control signals that temporally separate recombinant cell growth from recombinant protein production during fermentation. The activity of an expressed recombinant protein of the present invention may be improved by fragmenting, modifying, or derivatizing the resultant protein.

As used herein, amplifying the copy number of a nucleic acid sequence in a cell can be accomplished either by increasing the copy number of the nucleic acid sequence in the cell's genome or by introducing additional copies of the nucleic acid sequence into the cell by transformation. Copy number amplification is conducted in a manner such that greater amounts of enzyme are produced, leading to enhanced conversion of substrate to product. For example, recombinant molecules containing nucleic acids of the present invention can be transformed into cells to enhance enzyme synthesis. Transformation can be accomplished using any process by which nucleic acid sequences are inserted into a cell. Prior to transformation, the nucleic acid sequence on the recombinant molecule can be manipulated to encode an enzyme having a higher specific activity.

In accordance with the present invention, recombinant cells can be used to produce a MEKK protein of the present invention by culturing such cells under conditions effective to produce such a protein, and recovering the protein. Effective conditions-to produce a protein include, but are not limited to, appropriate media, bioreactor, temperature, pH and oxygen conditions that permit protein production. An appropriate, or effective, medium refers to any medium in which a cell of the present invention, when cultured, is capable of producing a MEKK protein. Such a medium is typically an aqueous medium comprising assimilable carbohydrate, nitrogen and phosphate sources, as well as appropriate salts, minerals, metals and other nutrients, such as vitamins. The medium may comprise complex nutrients or may be a defined minimal medium.

A preferred cell to culture is a recombinant cell that is capable of expressing the MEKK protein, the recombinant cell being produced by transforming a host cell with one or more nucleic acid molecules of the present invention. Transformation of a nucleic acid molecule into a cell can be accomplished by any method by which a nucleic acid molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed nucleic acid molecules of the present invention can remain extrachromosomal or can integrate into one or more sites within a chromosome of the transformed (i.e., recombinant) cell in such a manner that their ability to be expressed is retained.

With respect to methods for producing the subject MEKK pplypeptide, a host cell transfected with a nucleic acid vector directing expression of a nucleotide sequence encoding the subject polypeptides can he cultured under appropriate conditions to allow expression of the peptide to occur. The cells may be harvested, lysed and the protein isolated. A cell culture includes host cells, media and other byproducts. Suitable media for cell culture are well known in the art. The recombinant MEKK polypeptide can be isolated from cell culture medium, host cells, or both using techniques known in the art for purifying proteins including ion-exchange chromatography, gel filtration chromatography, ultrafiltration, electrophoresis, and immunoaffinity purification with antibodies specific for such peptide. In a preferred embodiment, the recombinant MEKK polypeptide is a fusion protein containing a domain which facilitates its purification, such as GST fusion protein or poly(His) fusion protein.

Cells of the present invention can be cultured in conventional fermentation bioreactors, which include, but are not limited to, batch, fed-batch, cell recycle, and continuous fermentors. Culturing can also be conducted in shake flasks, test tubes, microtiter dishes, and petri plates. Culturing is carried out at a temperature, pH and oxygen content appropriate for the recombinant cell. Such culturing conditions are well within the expertise of one of ordinary skill in the art.

Depending on the vector and host system used for production, resultant MEKK proteins may either remain within the recombinant cell or be secreted into the fermentation medium. The phrase “recovering the protein” refers simply to collecting the whole fermentation medium containing the protein and need not imply additional steps of separation or purification. MEKK proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, chromatofocusing and differential solubilization.

Alternatively, a MEKK protein of the present invention can be produced by isolating the MEKK protein from cells or tissues recovered from an animal that normally express the MEKK protein. For example, a cell type, such as T cells, can be isolated from the thymus of an animal. MEKK protein can then be isolated from the isolated primary T cells using standard techniques described herein.

The availability of purified and recombinant MEKK polypeptides as described in the present invention facilitates the development of assays which can be used to screen for drugs, including MEKK homologs, which are either agonists or antagonists of the normal cellular function of the subject MEKK polypeptides, or of their role in the pathogenesis of cellular differentiation and/or proliferation, and disorders related thereto. In one embodiment, the assay evaluates the ability of a compound to modulate binding between a MEKK polypeptide and a molecule that interacts either upstream or downstream of the MEKK polypeptide in the a cellular signaling pathway. A variety of assay formats will suffice and, in light; of the present inventions, will be comprehended by a skilled artisan.

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity and/or bioavallability of the test compound can be generally ignored in the in vitro system, the assay instead being focused primarily on the effect of the drug on the molecular target as may be manifest in an alteration of binding affinity with upstream or downstream elements.

Accordingly, in an exemplary screening assay of the present invention, the compound of interest is contacted with proteins which may function upstream (including both activators and repressors of its activity such as, Ras, Rac, Cdc 42 or Rho or other Ras superfamily members) or to proteins or nucleic acids which may function downstream of the MEKK polypeptide, whether they are positively or negatively regulated by it. For convenience, such polypeptides of a signal transduction pathway which interact directly with MEKK will be referred to below as MEKK-binding proteins (MEKK-bp). These proteins include the downstream targets of MEKKs, namely, members of the MAP kinase kinase family (MEKs or MKKs), as MEK1, MEK2, MKK1, MKK2, the stress-activated kinases (SEKs), also known as the Jun kinase kinases (JNKKs), MEKK3 and MEKK4 or the like. Other downstream targets of the MEKK family can include proteins from the mammalian MAP kinase family which includes, for example, the extracellular signal-regulated protein kinases (ERKs), c-Jun NH₂-terminal kinases (JNKs, or SAPKs), and the so-called “p38 subgroup” kinases (p38 kinases).

To the mixture of the compound and the MEKK-bp is then added a composition containing a MEKK polypeptide. Detection and quantification of complexes including MEKK and the MEKK-bp provide a means for determining a compound's :efficacy at inhibiting (or potentiating) complex formation between MEKK and the MEKK-binding protein. The efficacy of the compound can be assessed by generating dose response curves from data obtained using various concentrations of the test compound. Moreover, a control assay can also be performed to provide a baseline for comparison. In the control assay, isolated and purified MEKK polypeptide is added to a composition containing the MEKK-binding protein, and the formation of a complex is quantitated in the absence of the test compound.

Complex formation between the MEKK polypeptide and a MEKK-binding protein may be detected by a variety of techniques. Modulation of the formation of complexes can be quantitated using, for example, detectably labeled proteins such as radiolabeled, fluorescently labeled, or enzymatically labeled MEKK polypeptides, by immunoassay, or by chromatographic detection.

Typically, it will be desirable to immobilize either MEKK or its binding protein to facilitate separation of complexes from uncomplexed forms of one or both of the proteins, as well as to accommodate automation of the assay. Binding of the two proteins, in the presence and absence of a candidate agent, can be accomplished in any vessel suitable for containing the reactants. Examples include microtitre plates, test tubes, and micro-centrifuge tubes. In one embodiment, a fusion protein can be provided which adds a domain that allows the protein to be bound to a matrix. For example, glutathione-S-transferase/MEKK (GST/MEKK) fusion proteins can be adsorbed onto glutathione sepharose beads (Sigma Chemical, St. Louis, Mo.) or glutathione derivatized microtitre plates, which are then combined with the MEKK-bp, e.g., an ³⁵S-labeled, and the test compound, and the mixture incubated under conditions conducive to complex formation, e.g., at physiological conditions for salt and pH, though slightly more stringent conditions may be desired. Following incubation, the beads are washed to remove any unbound label, and the matrix immobilized and radiolabel determined directly.(e.g., beads placed in scintilant), or in the supernatant after the complexes are subsequently dissociated. Alternatively, the complexes can be dissociated from the matrix, separated by SDS-PAGE, and the level of MEKK-binding protein found in the bead fraction quantitated from the gel using standard electrophoretic techniques such as described in the appended examples.

Other techniques for immobilizing proteins on matrices are also available for use in the subject assay. For instance, either MEKK or its cognate binding protein can be immobilized utilizing conjugation of biotin and streptavidin. For instance, biotinylated MEKK molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce, Chemical). Alternatively, antibodies reactive with MEKK but which do not interfere with binding of upstream or downstream elements can be derivatized to the wells of the plate, and MEKK trapped in the wells by antibody conjugation. As above, preparations of a MEKK-binding protein and a test compound are incubated in the MEKK-presenting wells of the plate, and the amount of complex trapped in the well can be quantitated. Exemplary methods for detecting such complexes, in addition to those described above for the GST-immobilized complexes, include immunodetection of complexes using antibodies reactive with the MEKK binding protein, or which are reactive with the MEKK protein and compete with the binding protein; as well as enzyme-linked assays which rely on detecting an enzymatic activity associated with the binding protein, either intrinsic or extrinsic activity. In the instance of the latter, the enzyme can be chemically conjugated or provided as a fusion protein with the MEKK-bp. To illustrate, the MEKK-bp can be chemically cross-linked or genetically fused with horseradish peroxidase, and the amount of polypeptide trapped in the complex can be assessed with a chromogenic substrate of the enzyme, e.g., 3,3′-diamino-benzadine terahydrochloride or 4-chloro-1-napthol. Likewise, a fusion protein comprising the polypeptide and glutathione-S-transferase can be provided, and complex formation quantitated by detecting the GST activity using 1-chloro-2,4-dinitrobenzene (Habig et al (1974) J Biol Chem 249:7130).

For processes which rely on immunodetection for quantitating one of the proteins trapped in the complex, antibodies against the protein, such as anti-MEKK antibodies, can be used. Alternatively, the protein to be detected in the complex can be “epitope tagged” in the form of a fusion protein which includes, in addition to the MEKK sequence, a second polypeptide for which antibodies are readily available (e.g., from commercial sources). For instance, the GST fusion proteins described above can also be used for quantification of binding using antibodies against the GST moiety. Other useful epitope tags include myc-epitopes (e.g., see Ellison et al. (1991) J Biol Chem 266:21150-21157) which includes a 10-residue sequence from c-myc, as well as the pFLAG system (International Biotechnologies, Inc.) or the pEZZ-protein A system (Pharamacia, NJ).

In addition to cell-free assays, such as described above, the readily available source of vertebrate MEKK proteins provided by the present invention also facilitates the generation of cell-based assays for identifying small molecule agonists/antagonists and the like. Cells which are sensitive to MEKK-mediated signal transduction events can be caused to overexpress a recombinant MEKK protein in the presence and absence of a test agent of interest, with the assay scoring for modulation in MEKK-dependent responses by the target cell mediated by the test agent. As with the cell-free assays, agents which produce a statistically significant change in MEKK-dependent signal transduction (either inhibition or potentiation) can be identified.

In another embodiment of a drug screening, a two hybrid assay can be generated with a MEKK and MEKK-binding protein. This assay permits the detection of protein-protein interactions in yeast such that drug dependent inhibition or potentiation of the interaction can be scored. As an illustrative example, GAL4 protein is a potent activator of transcription in yeast grown on galactose. The ability of GAL4 to activate transcription depends on the presence of an N-terminal sequence capable of binding to a specific DNA sequence (UASG) and a C-terminal domain containing a transcriptional activator. A sequence encoding a MEKK protein, “A”, may be fused to that encoding the DNA binding domain of the GAL4 protein. A second hybrid protein may be created by fusing sequence encoding the GAL4 transactivation domain to sequence encoding a MEKK-bp, “B”. If protein “A” and protein “B” interact, that interaction serves to bring together the two domains of GAL4 necessary to activate transcription of a UASG-containing gene. In addition to co-expressing plasmids encoding both hybrid proteins, yeast strains appropriate for the detection of protein-protein interactions would contain, for example, a GAL1-lacZ fusion gene to permit detection of transcription from a UASG sequence. Other examples of two-hybrid assays or interaction trap assays are known in the art.

In an illustrative embodiment, a portion of MEKK4 providing a Rac/Cdc42 binding site is provided in one fusion protein, along with a second fusion protein including a Rac/Cdc42 polypeptide. This embodiment of the subject assay permits the screening of compounds which inhibit or potentiate the binding of MEKK4 and Cdc42.

Phosphorylation assays may also be used. MEKK binding proteins can be tested for their ability to phosphorylate substrates in addition, compounds that inhibit or activate MEKK regulated pathways and phenotypic responses can be tested.

Furthermore, each of the assay systems set out above can be generated in a “differential” format. That is, the assay format can provide information regarding specificity as well as potency. For instance, side-by-side comparison of a test compound's effect on different MEKKs can provide information on selectivity, and, permit the identification of compounds which selectively modulate the bioactivity of only a subset of the MEKK family.

The present invention also includes a method to identify compounds capable of regulating signals initiated from a receptor on the surface of a cell, such signal regulation involving in some respect, MEKK protein. Such a method comprises the steps of: (a) contacting a cell containing a MEKK protein with a putative regulatory compound; (b) contacting the cell with a ligand capable of binding to a receptor on the surface of the cell; and (c) assessing the ability of the putative regulatory compound to regulate cellular signals by determining activation of a member of a MEKK-dependent pathway of the present invention. A preferred method to perform step (c) comprises measuring the phosphorylation of a member of a MEKK-dependent pathway. Such measurements can be performed using immunoassays having antibodies specific for phosphotyrosines, phosphoserines and/or phosphothreonines. Another preferred method to perform step (c) comprises measuring the ability of the MEKK protein to phosphorylate a substrate molecule comprising a protein including MKK1, MKK2, MKK3, or MKK4, Raf-1, Ras-GAP and neurofibromin using methods described herein. Preferred substrates include MEK1, MEK2, JNKK1 and JNKK2. Yet another preferred method to perform step (c) comprises determining the ability of MEKK protein to bind to Ras, rac or Cdc 42 protein. In particular, determining the ability of MEKK protein to bind to GST-Ras^{V12}(GTPγS) or GSTRac^{V14}(GTPγS).

Putative compounds as referred to herein include, for example, compounds that are products of rational drug design, natural products and compounds having partially defined signal transduction regulatory properties. A putative compound can be a protein-based compound, a carbohydrate-based compound, a lipid-based compound, a nucleic acid-based compound, a natural organic compound, a synthetically derived organic compound, an anti-idiotypic antibody and/or catalytic antibody, or fragments thereof. A putative regulatory compound can be obtained, for example, from libraries of natural or synthetic compounds, in particular from chemical or combinatorial libraries (i.e., libraries of compounds that differ in sequence or size but that have the same building blocks; see for example, U.S. Pat. Nos. 5,010,175 and 5,266,684 of Rutter and Santi) or by rational drug design.

Preferred MEKK protein for use with the method includes recombinant MEKK protein. More preferred MEKK protein includes at least a portion of a MEKK protein having a kinase domain or apoptotic domain of MEKK.

Another aspect of the present invention includes a kit to identify compounds capable of regulating signals initiated from a receptor on the surface of a cell, such signals involving in some respect, MEKK protein. Such kits include: (a) at least one cell containing MEKK protein; (b) a ligand capable of binding to a receptor on the surface of the cell; and (c) a means for assessing the ability of a putative regulatory compound to alter phosphorylation of the MEKK protein. Such a means for detecting phosphorylation include methods and reagents known to those of skill in the art, for example, phosphorylation can be detected using antibodies specific for phosphorylated amino acid residues, such as tyrosine, serine and threonine. Using such a kit, one is capable of determining, with a fair degree of specificity, the location along a signal transduction pathway of particular pathway constituents, as well as the identity of the constituents involved in such pathway, at or near the site of regulation.

In another embodiment, a kit of the present invention can include: (a) MEKK protein; (b) MEKK substrate, such as MEKK and (c) a means for assessing the ability of a putative inhibitory compound to inhibit phosphorylation of the MEKK substrate by the MEKK protein.

In yet another embodiment, a mammalian MEKK gene can be used to rescue a yeast cell having a defective ste11 (or byr2) gene, such as a temperature sensitive mutant ste11 mutant (cf., Francois et al. (1991) J Biol Chem 266:6174-80; and Jenness et al. (1983) Cell 35:521-9). For example, a humanized yeast can be generated by amplifying the coding sequence of the human MEKK clone, and subcloning this sequence into a vector which contains a yeast promoter and termination sequences flanking the MEKK coding sequences. This plasmid can then be used to transform an ste11^{TS} mutant. To assay growth rates, cultures of the transformed cells can be grown at an permissive temperature for the TS mutant. Turbidity measurements, for example, can be used to easily determine the growth rate. At the non-permissive temperature, pheromone responsiveness of the yeast cells becomes dependent upon expression of the human MEKK protein. Accordingly, the humanized yeast cells can be utilized to identify compounds which inhibit the action of the human MEKK protein. It is also deemed to be within the scope of this invention that the humanized yeast cells of the present assay can be generated so as to comprise other human cell-cycle proteins. For example, human MEK and human MAPK can also be expressed in the yeast cell in place of ste7 and Fus3/Kss1. In this manner, the reagent cells of the present assay can be generated to more closely approximate the natural interactions which the mammalian MEKK protein might experience.

Furthermore, certain formats of the subject assays can be used to identify drugs which inhibit proliferation of yeast cells or other lower eukaryotes, but which have a substantially reduced effect on mammalian cells, thereby improving therapeutic index of the drug as an anti-mycotic agent. For instance, in one embodiment, the identification of such compounds is made possible by the use of differential screening assays which detect and compare drug-mediated disruption of binding between two or more different types of MEKK/MEKK-bp complexes, or which differentially inhibit the kinase activity of, for example, ste11 relative to a mammalian MEKK. Differential screening assays can be used to exploit the difference in drug-mediated disruption of human MEKK complexes and yeast ste11/byr2 complexes in order to identify agents which display a statistically significant increase in specificity for disrupting the yeast complexes (or kinase activity) relative to the human complexes. Thus, lead compounds which act specifically to inhibit proliferation of pathogens, such as fungus involved in mycotic infections, can be developed. By way of illustration, the present assays can be used to screen for agents which may ultimately be useful for inhibiting at least one fungus implicated in such mycosis as candidiasis, aspergillosis, mucomycosis, blastomycosis, geotrichosis, cryptococcosis, chromoblastoniycosis, coccidioidomycosis, conidiosporosis, histoplasmosis, maduromycosis, rhinosporidosis, nocaidiosis, para-actinomycosis, penicilliosis, monoliasis, or sporotrichosis. For example, if the mycotic infection to which treatment is desired is candidiasis, the present assay can comprise comparing the relative effectiveness of a test compound on mediating disruption of a human MEKK with its effectiveness towards disrupting the equivalent ste11/byr2 kinase from genes cloned from yeast selected from the group consisting of Candida albicans, Candida stellatoidea, Candida tropicalis, Candida parapsilosis, Candida krusei, Candida pseudotropicalis, Candida quillermondii, or Candida rugosa. Likewise, the present assay can be used to identify anti-fungal agents which may have therapeutic value in the treatment of aspergillosis by making use of genes cloned from yeast such as Aspergillus fumigatus, Aspergillus flavus, Aspergillus niger, Aspergillus nidulans, or Aspergillus terreus. Where the mycotic infection is mucormycosis, the complexes can be derived from yeast such as Rhizopus arrhizus, Rhizopus oryzae, Absidia corymbifera, Absidia ramosa, or Mucor pusillus. Sources of other ste11/byr2homologs for comparison with a human MEKK includes the pathogen Pneumocystis carinii.

Another aspect of the present invention relates to the treatment of an animal having a medical disorder that is subject to regulation or cure by manipulating a signal transduction pathway in a cell involved in the disorder. Such medical disorders include disorders which result from abnormal cellular growth or abnormal production of secreted cellular products. In particular, such medical disorders include, but are not limited to, cancer, autoimmune disease, inflammatory responses, allergic responses and neuronal disorders, such as Parkinson's disease and Alzheimer's disease. Preferred cancers subject to treatment using a method of the present invention include, but are not limited to, small cell carcinomas, non-small cell lung carcinomas with overexpressed EGF receptors, breast cancers with overexpressed EGF or Neu receptors, tumors having overexpressed growth factor receptors of established autocrine loops and tumors having overexpressed growth factor receptors of established paracrine loops. According to the present invention, the term treatment can refer to the regulation of the progression of a medical disorder or the complete removal of a medical disorder (e.g., cure). Treatment of a medical disorder can comprise regulating the signal transduction activity of a cell in such a manner that a cell involved in the medical disorder no longer responds to extracellular stimuli (e.g., growth factors or cytokines), or the killing of a cell involved in the medical disorder through cellular apoptosis.

The present invention relates to a method of inducing and/or maintaining a differentiated state, enhancing survival, and/or promoting (or alternatively inhibiting) proliferation of a cell responsive to a growth factor, morphogen or other environmental cue which effects the cell through at least one signal transduction pathway which includes a MEKK protein. In general, the method comprises contacting the cells with an amount of an agent which significantly (statistical) modulates MEKK-dependent signaling by the factor. For instance, it is contemplated by the invention that, in light of the present finding of an apparently broad involvement of members of the MEKK protein family in signal pathways implicated in the formation of ordered spatial arrangements of differentiated tissues in vertebrates, the subject method could be used to generate and/or maintain an array of different vertebrate tissue both in vitro and in vivo. A “MEKK therapeutic,” whether inductive or anti-inductive with respect to signaling by a MEKK-dependent pathway, can be, as appropriate, any of the preparations described above, including isolated polypeptides, gene therapy constructs, antisense molecules, peptidomimetics or agents identified in the drug assays provided herein.

There are a wide variety of pathological cell proliferative conditions for which MEKK therapeutics of the present invention can be used in treatment. For instance, such agents can provide therapeutic benefits where the general strategy being the inhibition of an anomalous cell proliferation. Diseases that might benefit from this methodology include, but are not limited to various cancers and leukemias, psoriasis, bone diseases, fibroproliferative, disorders such as involving connective tissues, atherosclerosis and other smooth muscle proliferative disorders, as well as chronic inflammation.

In addition to proliferative disorders, the present invention contemplates the use of MEKK therapeutics for the treatment of differentiative disorders which result from, for example, de-differentiation of tissue which may (optionally) be accompanied by abortive reentry into mitosis, e.g., apoptosis. Such degenerative disorders include chronic neurodegenerative diseases of the nervous system, including Alzheimer's disease, Parkinson's disease, Huntington's chorea, amylotrophic lateral sclerosis and the like, as well as spinocerebellar degenerations. Other differentiative disorders include, for example, disorders associated with connective tissue, such as may occur due to de-differentiation of chondrocytes or osteocytes, as well as vascular disorders which involve de-differentiation of endothelial tissue and smooth muscle cells, gastric ulcers characterized by degenerative changes in glandular cells, and renal conditions marked by failure to differentiate, e.g., Wilm's tumors.

It will also be apparent that, by transient, use of modulators of MEKK pathways, in vivo reformation of tissue can be accomplished, e.g., in the development and maintenance of organs. By controlling the proliferative and differentiative potential for different cells, the subject MEKK therapeutics can be used to reform injured tissue, or to improve grafting and morphology of transplanted tissue. For instance, MEKK agonists and antagonists can be employed in a differential manner to regulate different stages of organ repair after physical, chemical or pathological insult. For example, such regimens can be utilized in repair of cartilage, increasing bone density, liver repair subsequent to a partial hepatectomy, or to promote regeneration of lung tissue in the treatment of emphysema.

Another aspect of the present invention concerns transgenic animals which are comprised of cells (of that animal) which contain a transgene of the present invention and which preferably (though optionally) express an exogenous MEKK protein in one or more cells in the animal. A MEKK transgene can encode the wild-type form of the protein, or can encode homologs thereof, including both agonists and antagonists, as well as antisense constructs. In preferred embodiments, the expression of the transgene is restricted to specific subsets of cells, tissues or developmental stages utilizing, for example, cis-acting sequences that control expression in the desired pattern. In the present invention, such mosaic expression of a MEKK protein can be essential for many forms of lineage analysis and can additionally provide a means to assess the effects of, for example, lack of MEKK expression which might grossly alter development in small patches of tissue within an otherwise normal embryo. Toward this and, tissue-specific regulatory sequences and conditional regulatory sequences can be used to control expression of the transgene in certain spatial patterns. Moreover, temporal patterns of expression can be provided by, for example, conditional recombination systems or prokaryotic transcriptional regulatory sequences.

Genetic techniques which allow for the expression of transgenes can be regulated via site-specific genetic manipulation in vivo are known to those skilled in the art. For instance, genetic systems are available which allow for the regulated expression of a recombinase that catalyzes the genetic recombination a target sequence. As used herein, the phrase “target sequence” refers to a nucleotide sequence that is genetically recombined by a recombinase. The target sequence is flanked by recombinase recognition sequences and is generally either excised or inverted in cells expressing recombinase activity. Recombinase catalyzed recombination events can be designed such that recombination of the target sequence results in either the activation or repression of expression of one of the subject MEKK proteins. For example, excision of a target sequence which interferes with the expression of a recombinant MEKK gene, such as one which encodes an antagonistic homolog or an antisense transcript, can be designed to activate expression of that gene. This interference with expression of the protein can result from a variety of mechanisms, such as spatial separation of the MEKK gene from the promoter element or an internal stop codon. Moreover, the transgene can be made wherein the coding sequence of the gene is flanked by recombinase recognition sequences and is initially transfected into cells in a 3′ to 5′ orientation with respect to the promoter element. In such an instance, inversion of the target sequence will reorient the subject gene by placing the 5′ end of the coding sequence in an orientation with respect to the promoter element which allow for promoter driven transcriptional activation.

In an illustrative embodiment, either the crelloxP recombinase system of bacteriophage P1 (Lakso et al. (1992) PNAS 89:6232-6236; Orban et al. (1992) PNAS 89:6861-6865) or the FLP recombinase system of Saccharomyces cerevisiae (O'Gorman. et al. (1991) Science 251:1351-1355; PCT publication WO 92/15694) can be used to generate in vivo site-specific genetic recombination systems. Cre recombinase catalyzes the site-specific recombination of an intervening target sequence located between loxP sequences. loxP sequences are 34 base pair nucleotide repeat sequences to which the Cre recombinase binds and are required for Cre recombinase mediated genetic recombination. The orientation of loxP sequences determines whether the intervening target sequence is excised or inverted when Cre recombinase is present (Abremski et al. (1984) J. Biol. Chem. 259:1509-1514); catalyzing the excision of the target sequence when the loxP sequences are oriented as direct repeats and catalyzes inversion of the target sequence when loxP sequences are oriented as inverted repeats.

Accordingly, genetic recombination of the target sequence is dependent on expression of the Cre recombinase. Expression of the recombinase can be regulated by promoter elements which are subject to regulatory control, e.g., tissue-specific, developmental stage-specific, inducible or repressible by externally added agents. This regulated control will result in genetic recombination of the target sequence only in cells where recombinase expression is mediated by the promoter element. Thus, the activation expression of a recombinant MEKK protein can:be regulated via control of recombinase expression;

Use of the cre/loxP recombinase system to regulate expression of a recombinant MEKK protein requires the construction of a transgenic animal containing transgenes encoding both the Cre recombinase and the subject protein. Animals containing both the Cre recombinase and a recombinant MEKK gene can be provided through the construction of “double” transgenic animals. A convenient method for providing such animals is to mate two transgenic animals each containing a transgene, e.g., a MEKK gene and recombinase gene.

One advantage derived from initially constructing transgenic animals containing a MEKK transgene in a recombinase-mediated expressible format derives from the likelihood that the subject protein, whether agonistic or antagonistic, can be deleterious upon expression in the transgenic animal. In such an instance, a founder population, in which the subject transgene is silent in all tissues, can be propagated and maintained. Individuals of this founder population can be crossed with animals expressing the recombinase in, for example, one or more tissues and/or a desired temporal pattern. Thus, the creation of a founder population in which, for example, an antagonistic MEKK transgene is silent will allow the study of progeny from that founder in which disruption of MEKK mediated induction in a particular tissue or at certain developmental stages would result in, for example, a lethal phenotype.

Similar conditional transgenes can be provided using prokaryotic promoter sequences which require prokaryotic proteins to be simultaneous expressed in order to facilitate expression of the MEKK transgene. Exemplary promoters and the corresponding trans-activating prokaryotic proteins are given in U.S. Pat. No. 4,833,080.

Moreover, expression of the conditional transgenes can be induced by gene therapy-like methods wherein a gene encoding the trans-activating protein, e.g., a recombinase or a prokaryotic protein, is delivered to the tissue and caused to be expressed, such as in a cell-type specific manner. By this method, a MEKK transgene could remain silent into adulthood until “turned on” by the introduction of the trans-activator.

In an exemplary embodiment, the “transgenic non-human animals” of the invention are produced by introducing transgenes into the germline of the non-human animal. Embryonic target cells at various developmental stages can be used to introduce transgenes. Different methods are used depending on the stage of development of the embryonic target cell. The zygote is the best target for micro-injection. In the mouse, the male pronucleus reaches the size of approximately 20 micrometers in diameter which allows reproducible injection of 1-2 pl of DNA solution. The use of zygotes as a target for gene transfer has a major advantage in that in most cases the injected DNA will be incorporated into the host gene before the first cleavage (Brinster et al. (1985) PNAS 82:4438-4442). As a consequence, all cells of the transgenic non-human animal will carry the incorporated transgene. This will in general also be reflected in the efficient transmission of the transgene to offspring of the founder since 50% of the germ cells will harbor the transgene. Microinjection of zygotes is the preferred method for incorporating transgenes in practicing the invention.

Retroviral infection can also be used to introduce MEKK transgenes into a non-human animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J. 6:383-388). Alternatively, infection can be performed at a later stage. Virus or virus-producing cells can be injected into the blastocoele (Jahner et al. (1982) Nature 298:623-628). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1982) supra).

A third type of target cell for transgene introduction is the embryonic stem cell (ES). ES cells are obtained from pre-implantation embryos cultured in vitro and fused with embryos (Evans et al. (1981) Nature 292:154-156; Bradleyet al. (1984) Nature 309:255-258; Gossler et al. (1986) PNAS 83: 9065-9069; and Robertson et al. (1986) Nature 322:445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240:1468-1474.

Methods of making MEKK knock-out or disruption transgenic animals are also generally known. See, for example, Manipulating the Mouse Embryo, (Cold Spring Harbor Laboratory Press, Cold Spring Harbor, N.Y., 1986). Recombinase dependent knockouts can also be generated, e.g., by homologous recombination to insert recombinase target sequences flanking portions of an endogenous MEKK gene, such that tissue specific and/or temporal control of inactivation of a MEKK allele can be controlled as above.

According to the present invention, an isolated, or biologically pure, peptide, is a peptide that has been removed from its natural milieu. As such, “isolated” and “biologically pure” do not necessarily reflect the extent to which the protein has been purified. An isolated compound of the present invention can be obtained from a natural source or produced using recombinant DNA technology or chemical synthesis. As used herein, an isolated peptide can be a full-length protein or any homolog of such a protein in which amino acids have been deleted (e.g., a truncated version of the protein), inserted, inverted, substituted and/or derivatized (e.g., by glycosylation, phosphorylation, acetylation, myristylation, prenylation, palmitoylation, and/or amidation) such that the peptide is capable of regulating the binding of Ras superfamily protein to MEKK protein.

In accordance with the present invention, a “mimetope” refers to any compound that is able to mimic the ability of an isolated compound of the present invention. A mimetope can be a peptide that has been modified to decrease its susceptibility to degradation but that still retain regulatory activity. Other examples of mimetopes include, but are not limited to, protein-based compounds, carbohydrate-based compounds, lipid-based compounds, nucleic acid-based compounds, natural organic compounds, synthetically derived organic compounds, anti-idiotypic antibodies and/or catalytic antibodies, or fragments thereof. A mimetope can be obtained by, for example, screening libraries of natural and synthetic compounds as disclosed herein that are capable of inhibiting the binding of Ras superfamily protein to MEKK. A mimetope can also be obtained by, for example, rational drug design. In a rational drug design procedure, the three-dimensional structure of a compound of the present invention can be analyzed by, for example, nuclear magnetic resonance (NMR) or x-ray crystallography. The three-dimensional structure can then be used to predict structures of potential mimetopes by, for example, computer modeling. The predicted mimetope structures can then be produced by, for example, chemical synthesis, recombinant DNA technology, or by isolating a mimetope from a natural source (e.g., plants, animals, bacteria and fungi).

The therapeutic methods of the present invention may also comprise injecting an area of a subject's body with an effective amount of a naked plasmid DNA compound (such as is taught, for example in Wolff et al., 1990, Science 247, 1465-1468). A naked plasmid DNA compound comprises a nucleic acid molecule encoding a MEKK protein of the present invention, operatively linked to a naked plasmid DNA vector capable of being taken up by and expressed in a recipient cell located in the body area. A preferred naked plasmid DNA compound of the present invention comprises a nucleic acid molecule encoding a truncated MEKK protein having deregulated kinase activity. Preferred naked plasmid DNA vectors of the present invention include those known in the art. When administered to a subject, a naked plasmid DNA compound of the present invention transforms cells within the subject and directs the production of at least a portion of a MEKK protein or RNA nucleic acid molecule that is capable of regulating the apoptosis of the cell.

A naked plasmid. DNA compound of the present invention is capable of treating a subject suffering from a medical disorder including cancer, autoimmune disease, inflammatory responses, allergic responses and neuronal disorders, such as Parkinson's disease and Alzheimer's disease. For example, a naked plasmid DNA compound can be administered as an anti-tumor therapy by injecting an effective amount of the plasmid directly into a tumor so that the plasmid is taken up and expressed by a tumor cell, thereby killing the tumor cell. As used herein, an effective amount of a naked plasmid DNA to administer to a subject comprises an amount needed to regulate or cure a medical disorder the naked plasmid DNA is intended to treat, such mode of administration, number of doses and frequency of dose capable of being decided upon, in any given situation, by one of skill in the art without resorting to undue experimentation.

An isolated compound of the present invention can be used to formulate a therapeutic composition. In one embodiment, a therapeutic composition of the present invention includes at least one isolated peptide of the present invention. A therapeutic composition for use with a treatment method of the present invention can further comprise suitable excipients. A therapeutic compound for use with a treatment method of the present invention can be formulated in an excipient that the subject to be treated can tolerate. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful excipients include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosal, m- or o-cresol, formalin and benzyl alcohol. Standard formulations can either be liquid injectables or solids which can be taken up in a suitable liquid as a suspension or solution for injection. Thus, in a non-liquid formulation, the excipient can comprise dextrose, human serum albumin, preservatives, etc., to which sterile water or saline can be added prior to administration.

In another embodiment, a therapeutic compound for use with a treatment method of the present invention can also comprise a carrier. Carriers are typically compounds that increase the half-life of a therapeutic compound in the treated animal. Suitable carriers include, but are not limited to, liposomes, micelles, cells, polymeric controlled release formulations, biodegradable implants, bacteria, viruses, oils, esters, and glycols. Preferred carriers include liposomes and micelles.

A therapeutic compound for use with a treatment method of the present invention can be administered to any subject having a medical disorder as herein described. Acceptable protocols by which to administer therapeutic compounds of the present invention in an effective manner can vary according to individual dose size, number of doses, frequency of dose administration, and mode of administration. Determination of such protocols can be accomplished by those skilled in the art without resorting to undue experimentation. An effective dose refers to a dose capable of treating a subject for a medical disorder as described herein. Effective doses can vary depending upon, for example, the therapeutic compound used, the medical disorder being treated, and the size and type of the recipient animal. Effective doses to treat a subject include doses administered over time that are capable of regulating the activity, including growth, of cells involved in a medical disorder. For example, a first dose of a naked plasmid DNA compound of the present invention can comprise an amount that causes a tumor to decrease in size by about 10% over 7 days when administered to a subject having a tumor. A second dose can comprise at least the same the same therapeutic compound than the first dose.

Another aspect of the present invention includes a method for prescribing treatment for subjects having a medical disorder as described herein. A preferred method for prescribing treatment comprises: (a) measuring the MEKK protein activity in a cell involved in the medical disorder to determine if the cell is susceptible to treatment using a method of the present invention; and (b) prescribing treatment comprising regulating the activity of a MEKK-dependent pathway relative to the activity of a Raf-dependent pathway in the cell to induce the apoptosis of the cell. The step of measuring MEKK protein activity can comprise: (1) removing a sample of cells from a subject; (2) stimulating the cells with a TNFα; and (3) detecting the state of phosphorylation of MKK3, MKK4 or JNKK protein using an immunoassay using antibodies specific for phosphothreonine and/or phosphoserine.

The present invention also includes antibodies capable of selectively binding to a MEKK protein of the present invention. Such an antibody is herein referred to as an anti-MEKK antibody. Polyclonal populations of anti-MEKK antibodies can be contained in a MEKK antiserum. MEKK antiserum can refer to affinity purified polyclonal antibodies, ammonium sulfate cut antiserum or whole antiserum. As used herein, the term “selectively binds to” refers to the ability of such an antibody to preferentially bind to MEKK proteins. Binding can be measured using a variety of methods known to those skilled in the art including immunoblot assays, immunoprecipitation assays, enzyme immunoassays (e.g., ELISA), radioimmunoassays, immunofluorescent antibody assays and immunoelectron microscopy; see, for example, Sambrook et al., Molecular Cloning: A Laboratory Manual, Cold Spring Harbor Labs Press, 1989.

Antibodies of the present invention can be either polyclonal or monoclonal antibodies and can be prepared using techniques standard in the art. Antibodies of the present invention include functional equivalents such as antibody fragments and genetically-engineered antibodies, including single chain antibodies, that are capable of selectively binding to at least one of the epitopes of the protein used to obtain the antibodies. Preferably, antibodies are raised in response to proteins that are encoded, at least in part, by a MEKK nucleic acid molecule. More preferably antibodies are raised in response to at least apportion of a MEKK protein, and even more preferably antibodies are raised in response to either the amino terminus or the carboxyl terminus of a MEKK protein. Preferably, an antibody of the present invention has a single site binding affinity of from about 10³M⁻¹ to about 10¹²M⁻¹ for a MEKK protein of the present invention.

A preferred method to produce antibodies of the present invention includes administering to an animal an effective amount of a MEKK protein to produce the antibody and recovering the antibodies. Antibodies of the present invention have a variety of potential uses that are within the scope of the present invention. For example, such antibodies can be used to identify unique MEKK proteins and recover MEKK proteins.

Another aspect of the present invention comprises a therapeutic compound capable of regulating the activity of a MEKK-dependent pathway in a cell identified by a process, comprising: (a) contacting a cell with a putative regulatory molecule; and (b) determining the ability of the putative regulatory compound to regulate the activity of a MEKK-dependent pathway in the cell by measuring the activation of at least one member of said MEKK-dependent pathway. Preferred methods to measure the activation of a member of a MEKK-dependent pathway include measuring the transcription regulation activity of c-Myc protein, measuring the phosphorylation of a protein selected from the group consisting of MEKK, JNKK, JNK, Jun, ATF-2, Myc, and combinations thereof

The foregoing description of the invention has been presented for purposes of illustration and description. Further, the description is not intended to limit the invention to the form disclosed herein. Consequently, variations and modifications commensurate with the above teachings, and the skill or knowledge in the relevant art are within the scope of the present invention. The preferred embodiment described herein above is further intended to explain the best mode known of practicing the invention and to enable others skilled in the art to utilize the invention in various embodiments and with various modifications required by their particular applications or uses of the invention. It is intended that the appended claims be construed to include alternate embodiments to the extent permitted by the prior art.

### EXAMPLES

The following examples describe the isolation and cloning of a human and murine MEKK1 nucleic acid molecule as and characterize the encoded MEKK1 proteins as well as apoptotic fragments of MEKK1 proteins. Additional exemplification of MEKK1 proteins and activities can be found in U.S. Pat. Nos. 5,405,941, 5,854,043, and 5,753,446, in published PCT international application Nos. WO 94/24159 and W095/28421, as well as in the following publications:
Russell et al. (1995) Journal of Biological Chemistry 270(20):11757-11760
Lin et al. (1995) Science 268:286-290
Johnson et al. (1996) Journal of Biological Chemistry 271(6):3229-3237
Gardner et al. (1994) Molecular Biology of the Cell 5:193-201
Blumer et al. (1994) Proc. Natl. Acad. Sci. USA 91:4925-4929
Johnson (1995) U.S. Pat. No. 5,405,941
Lange-Carter et al. (1993) Science 260:315-319
Lange-Carter et al. (1994) Science 265:1458-1461
Minden et al. (1994) Science 266:1719-1723.

### Example 1

Isolation and Cloning of Human and Murine MEKK1 Proteins.

MEKK1 Nucleotide Sequences

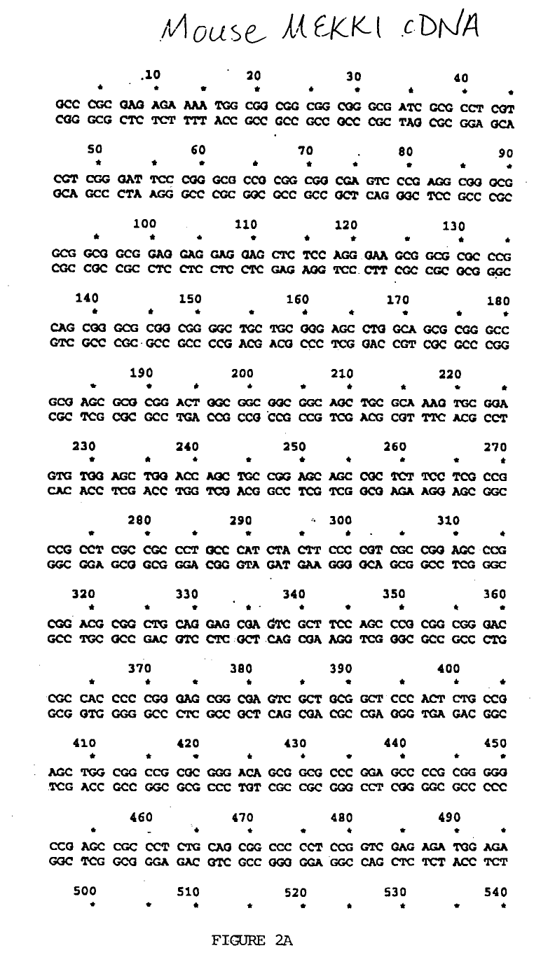

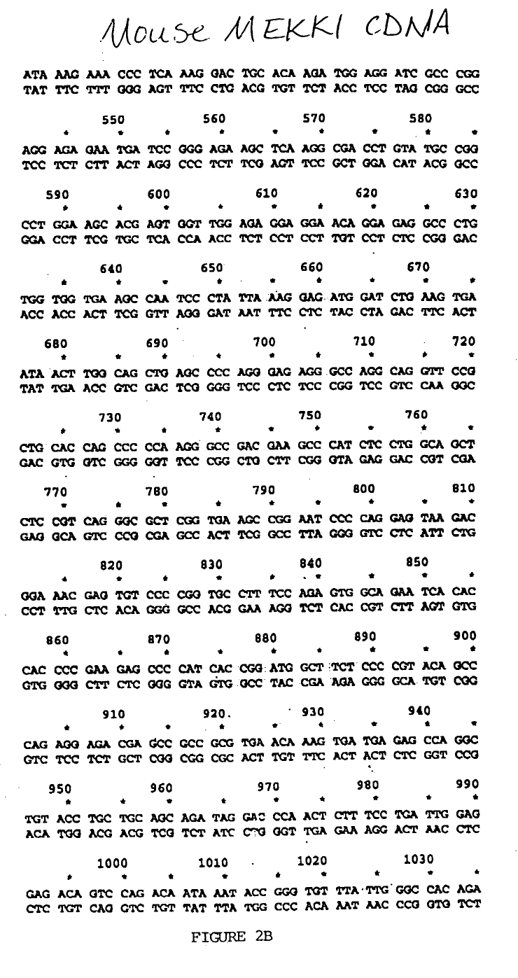

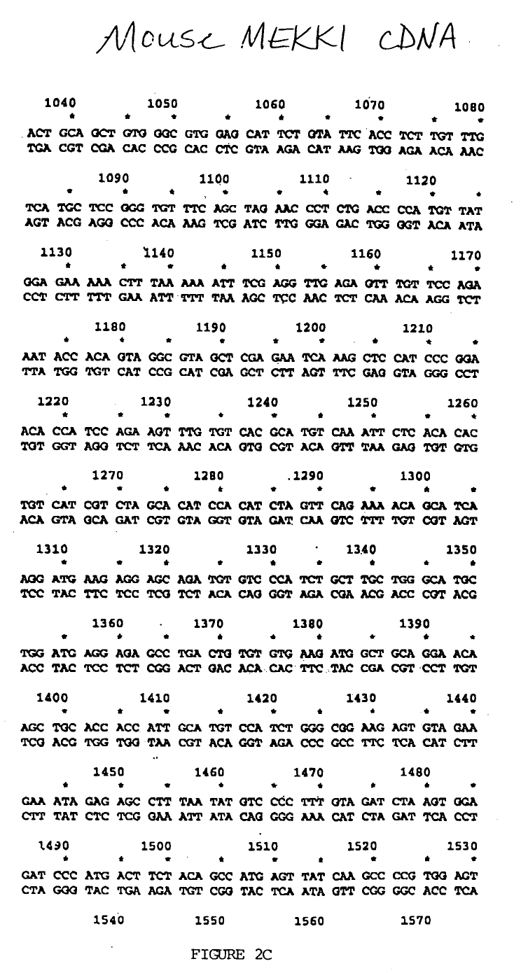

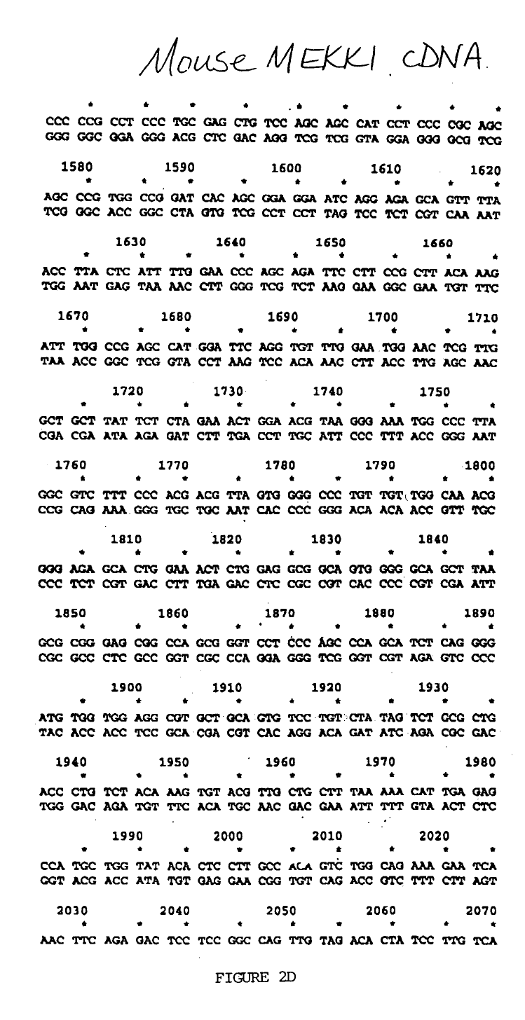

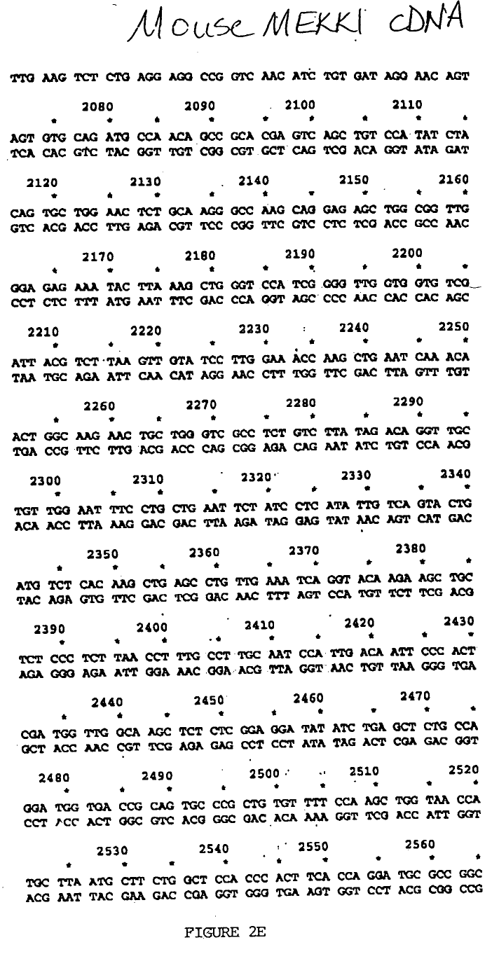

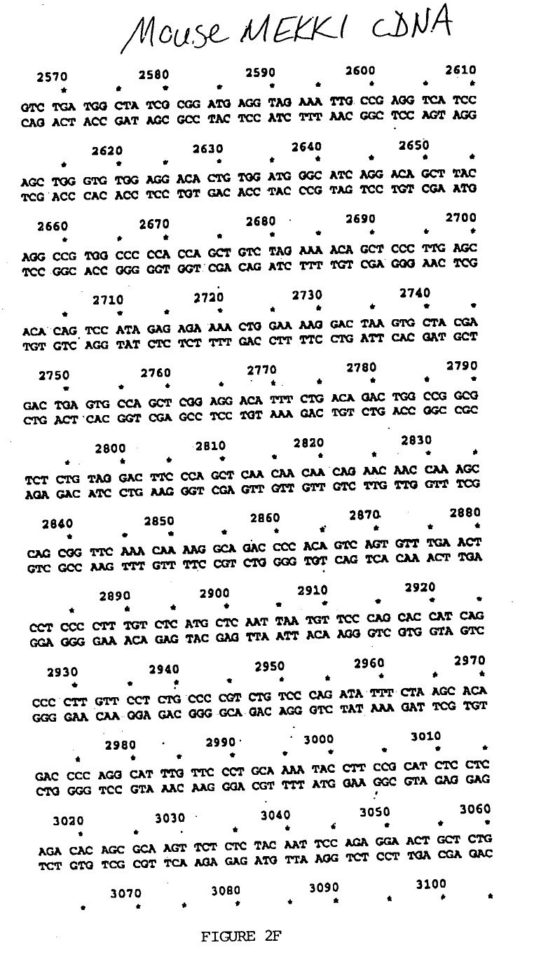

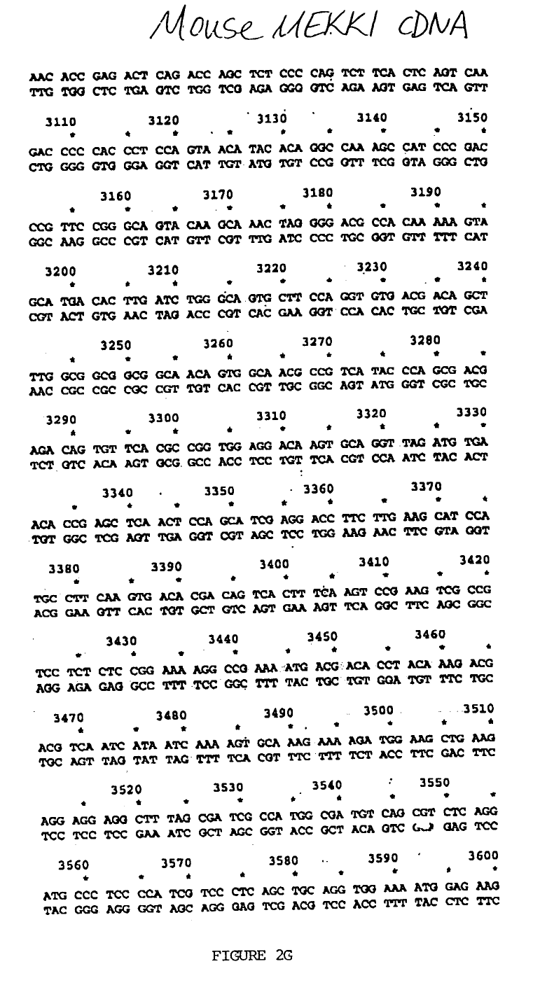

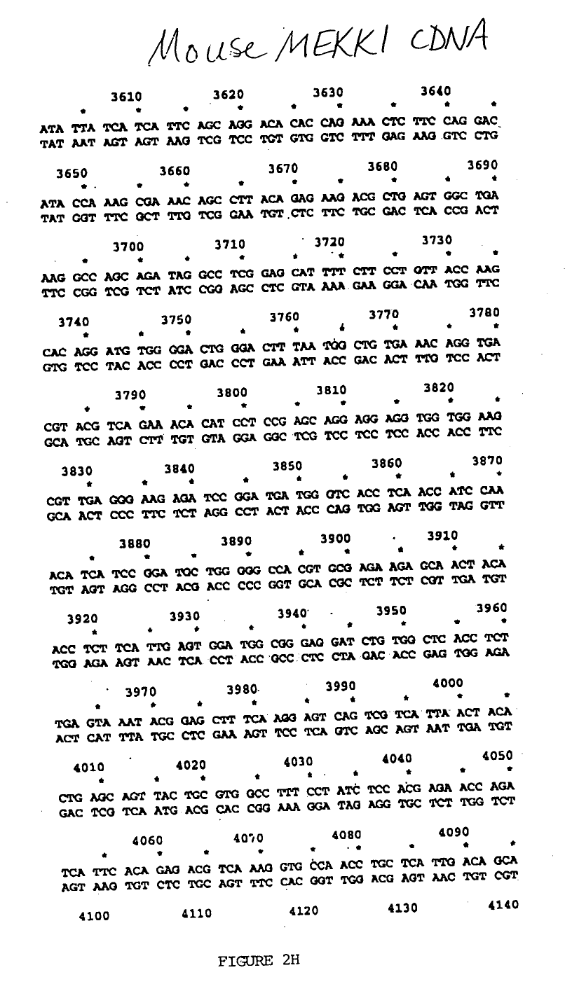

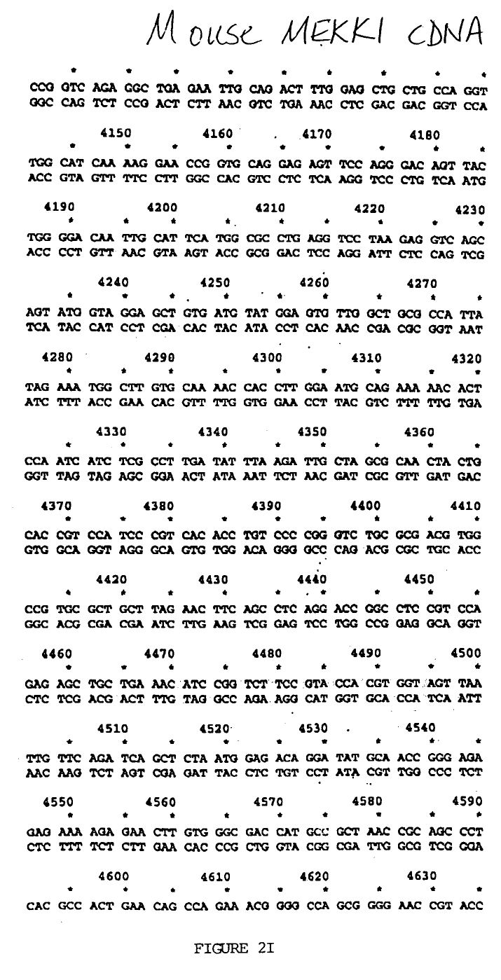

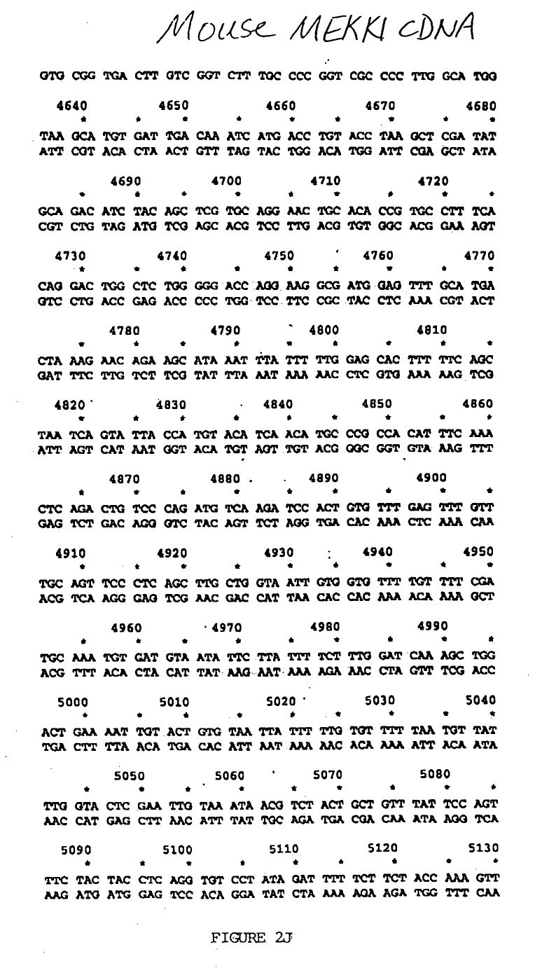

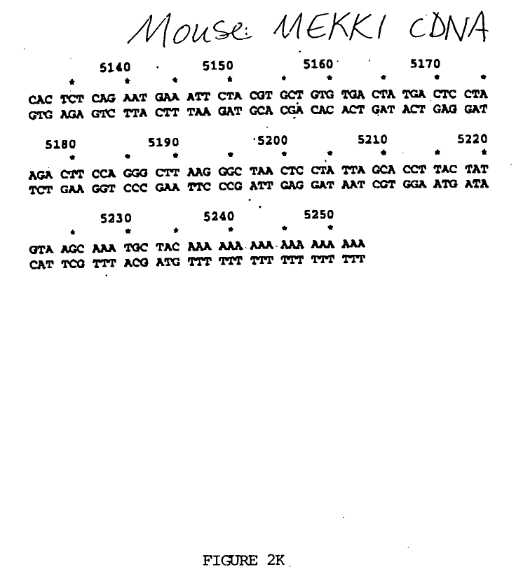

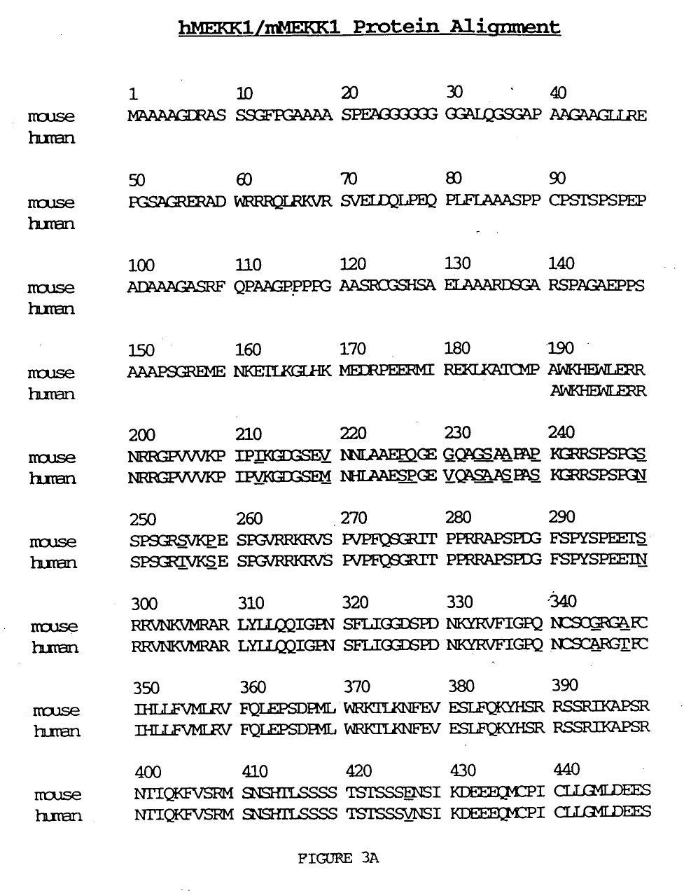

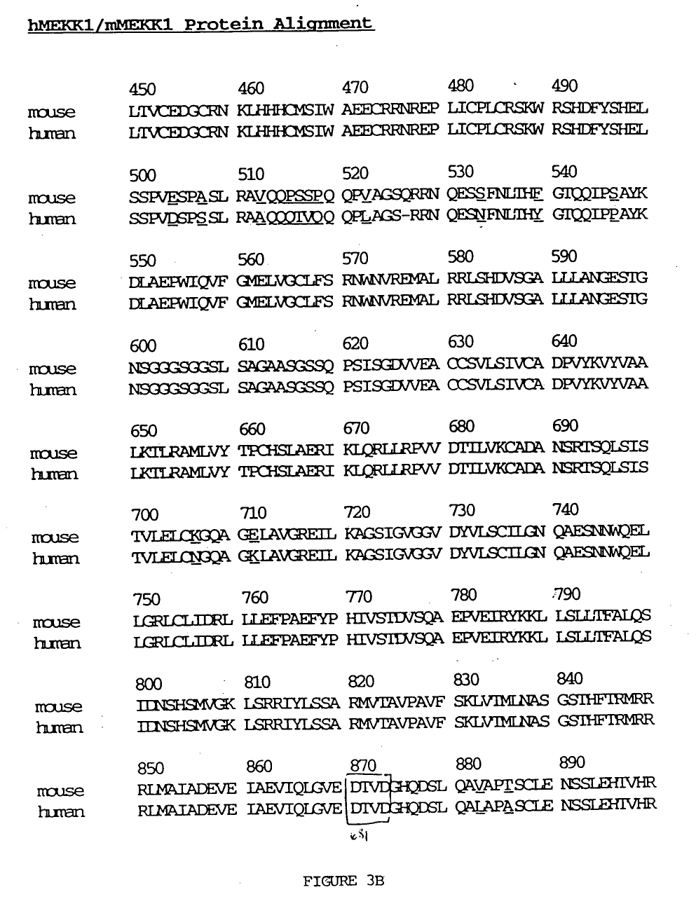

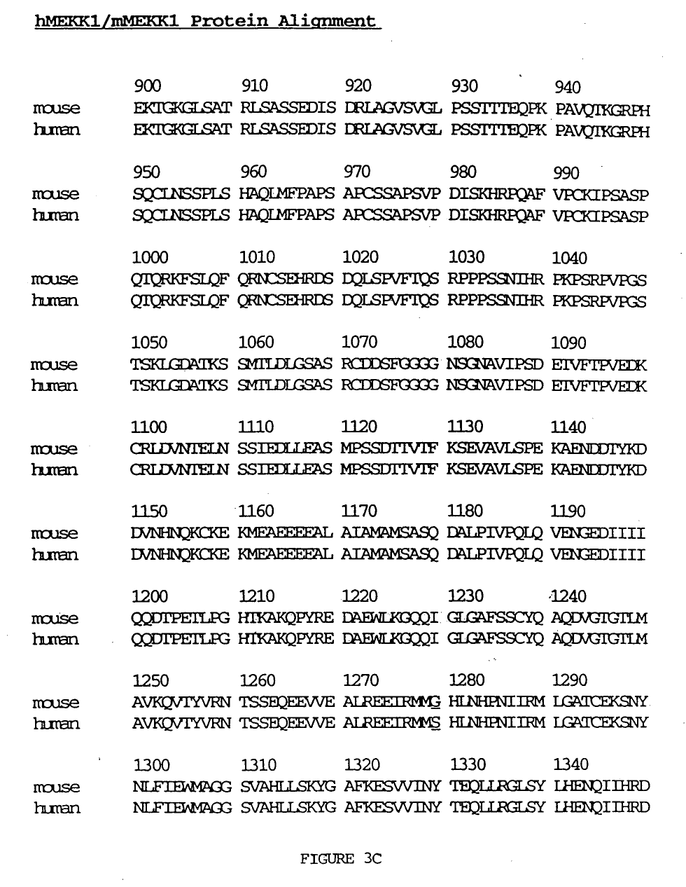

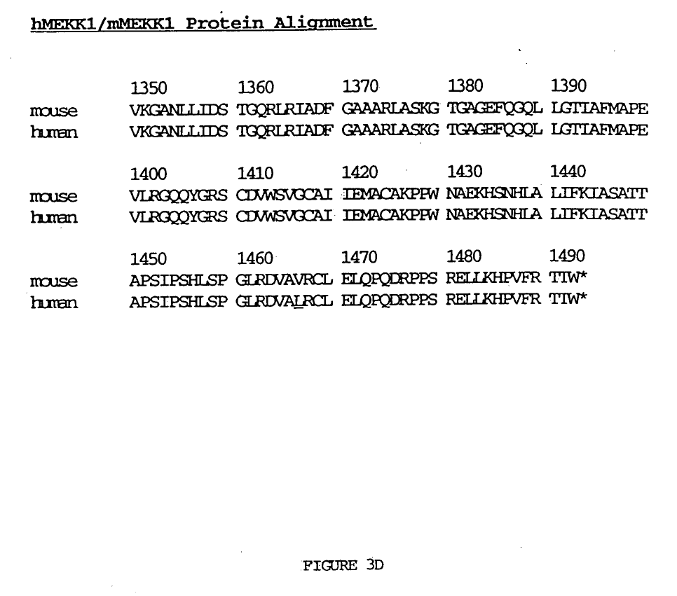

A partial murine MEKK1 nucleotide sequences, and encoded protein, was cloned and has previously been described in U.S. Pat. No. 5,405,941, which is incorporated herein by this reference. The partial murine MEKK1 nucleotide sequence is shown in SEQ ID NO: 1. The predicted amino acid sequence is shown in SEQ ID NO2. Additional cloning based on the sequence of the partial murine MEKK1 shown in SEQ ID NO:1 resulted in the nucleotide sequence of a full-length murine MEKK1 DNA which is set forth in FIG. 2 and as SEQ ID NO:3. The predicted amino acid sequence of full-length murine MEKK1 is set forth as SEQ ID NO:4.

Cloning of Human MEKK1

cDNA Preparation—Total mRNA was extracted and isolated from T47D cells using 1×10⁷ cells per purification in the QuickPrep Micro mRNA Purification Kit (Pharmacia). First strand cDNA was produced using 33 microliters of the purified mRNA per reaction in the Ready-to-Go T-Primed First-Strand Kit (Pharmacia).

PCR Amplification—The sense strand primer 5′-GAACACCATCCAGAAGTTTG-3′ (SEQ ID NO:13), which was designed from the mouse MEKK1 (mMEKK1) cDNA sequence, was used in conjunction with the antisense primer 5′-CACTTTGTAGACAGGGTCAGC-3′ (SEQ ID NO: 14) in a polymerase chain reaction (PCR) using the first strand cDNA described above as a template (RT-PCR) to amplify the region from bases 1211-1950. Taq DNA Polymerase (Boehringer Mannheim) was used in a RT-PCR of 30 cycles (1 min. 94° C.; 1 min. 50° C.; 3 min., 72° C.), followed by a 10 min. incubation at 72° C. A band of approximately 800 bp was isolated by purification from a 1% agarose gel and ligated overnight at 14° C. into pGEM-T coli by heat shock at 42° C., and plated on Luria Broth (LB) plates containing ampicillin and X-gal. Colonies were screened by blue/white color selection, grown up in 5 ml of LB containing ampicillin, and the plasmid DNA was isolated using the Wizard Mini-pre Kit (Promega). Isolates were then screened for insert size by digesting with PstI and AatII (Promega), and running on a 1% agarose gel. Appropriately sized inserts were sequenced from both ends using T7 and SP6 vector primers. The resulting sequence was aligned to the known mMEKK1 sequence, and determined to be hMEKK1 by homology. In order to amplify the region from bases 2263-3743, the sense primer 5′-TGGGTCGCCTCTGTCTTATAGACAG-3′ (SEQ ID NO:15) was used in conjunction with the antisense primer 5′-CACATCCTGTGCTTGGTAAC-3′ (SEQ ID NO:16) in a RT-PCR of 30 cycles (1 min. 94° C.; 1 min., 50° C.; 2 min., 72° C.), followed by a 10 min. incubation at 72° C. A band of approximately 1.5 kb was isolated by purification from a 1% agarose gel, ligated, cloned, and sequenced as stated above. In order to amplify the 3′ region of hMEKK1 from bases 3304-4493, the sense primer 5′-AGGACAAGTGCAGGTTAGATG-3′ (SEQ ID NO:17) was used in a RT-PCR of 30 cycles (1 min., 94° C.; 1 min., 50° C.; 2 min., 72° C.), followed by a 10 min. incubation at 72° C. A band of approximately 1.3 kb was isolated by purification from a 1% agarose gel, ligated, cloned, and sequenced as stated above. Sequence was also confirmed for this clone using the internal sequencing primer 5′-GCTGTCCATATCTACAGTGCT-3′ (SEQ ID NO:18). In order to amplify the region from bases 580-1310, the sense primer 5′-CGGCCTGGAAGCACGAGTGGT-3′ (SEQ ID NO:19) was used in conjunction with the antisense primer 5′-TTCATCCTTGATGCTGTTTTC-3′ (SEQ ID NO:20) in a RT-PCR of 30 cycles (1 min., 94° C.; 1 min., 50° C.; 2 min., 72° C.), followed by a 10 min. incubation at 72° C. A band of approximately 700 bp was isolated by purification from a 1% agarose gel, ligated, cloned, and sequenced as stated above. The overlapping sequence data was compiled into a single contig using Sequencer 2.0 (Gene Codes), and aligned to the mMEKK1 sequence.

A BLAST search using the amino acid sequences of murine MEKK1 and human MEKK1 as described in this example reveals nucleotide and amino acid sequences having substantial homology to those set forth in SEQ ID NOs:3-6 (e.g., sequences having Accession No. 423499, Accession No. 2507203 and Accession No. U23470).

### Example 2

Apoptotic Fragments of MEKK1

This example demonstrates that MEK kinase 1 (MEKK1), a 196 kDa protein kinase, functions to integrate proteases and signal transduction pathways involved in the regulation of apoptosis. Cleavage of mouse MEKK1 at Asp⁸⁷⁴ generates a 91 kDa kinase fragment and a 113 kDa NH₂-terminal fragment. The kinase fragment of MEKK1 induces apoptosis. Cleavage of MEKK1 and apoptosis are inhibited by p35 and CrmA, viral inhibitors of the ICE/FLICE proteases that commit cells to apoptosis. Mutation of the MEKK1 sequence ⁸⁷¹DTVD⁸⁷⁴ (SEQ ID NO: 7), a cleavage site for CCP32-like proteases, to alanines inhibited proteolysis of MEKK1 and apoptosis induced by overexpression of MEKK1. Inhibition of MEKK1 proteolysis inhibited apoptosis but did not block MEKK1 stimulation of c-Jun kinase activity, indicating that c-Jun kinase activation was not sufficient for apoptosis. During the apoptotic response to UV irradiation, cisplatin, etoposide and mitomycin C, MEKK1 undergoes a phosphorylation-dependent activation followed by its proteolysis. These results show that MEKK1 activation and cleavage occurs in response to genotoxic agents and the activated kinase fragment functions to commit cells to apoptosis.

Publications referred to in these examples are abbreviated using the first author's name and the year of publication. A list of the full citation of each publication referred to in this example is provided at the end of the example.

Apoptosis or programmed cell death is a physiological process important in differentiation and tissue modeling (Williams and Smith, 1993; Steller, 1995). Apoptosis can be triggered by many different stimuli including growth factor deprivation (Xia et al., 1995; Park et al., 1996), exposure of specific cell types to cytokines such as TNFα and Fas ligand (Vandenabeele et al., 1995; Kägi et al., 1994; Lowin et al., 1994), virus infection (Esolen et al., 1995; Hinshaw et al., 1994; Terai et al., 1991; Tyler et al., 1995), and DNA damaging agents including irradiation and chemicals that induce DNA adducts (Canman and Kastan, 1996). Proteases of the ICE/FLICE family are activated during the apoptotic response that cleave specific protein substrates resulting in an irreversible commitment to cell death. Several ICE/FLICE substrates have been identified including poly (ADP-ribose) polymerase (Lazebnik et al., 1994), U1 small nuclear ribonucleoprotein (Casciola-Rosen et ad, 1994), lamin (Lazebnik et al., 1995), D4-GDI (Na et al., 1996), fodrin (Cryns et al., 1996), protein kinase Cδ (Emoto et al., 1995), sterol regulatory element binding protein (Wang et al., 1996), retinoblastoma protein (An and Dou, 1996), DNA-dependent protein kinase (Casciola-Rosen et al., 1995), and the proteases themselves (Orth et al., 1996).

Two ICE-like protease activities appear necessary for the apoptotic response, each with a specific substrate selectivity. ICE-like proteases such as Ced-3 have a specificity for proteins encoding the four amino acid sequence YVAD (SEQ ID NO: 10) (Howard et al., 1991) while CPP32-like proteases have a preference for the sequence DEVD (SEQ ID NO: 11) (Nicholson et al., 1995). Both groups of proteases cleave at the terminal aspartic acid residue of the recognition sequence. Several viruses encode proteins that are specific inhibitors of the ICE/FLICE proteases. Most notably CrmA is a poxvirus protein that inhibits ICE-like proteases, and p35 is a baculovirus protein that has broad inhibitory activity to ICE/FLICE-like proteases (Fraser and Evan, 1996; Clem et al., 1996). Expression of CrmA and p35 inhibit the apoptotic response to many different stimuli demonstrating the requirement of ICE/FLICE proteases during programmed cell death (Beidler et al., 1996; Los et al., 1995).

In addition to:ICE/FLICE proteases, it is becoming increasingly clear that signal transduction pathways involving specific protein kinases are involved in mediating apoptosis. Specifically, the c-Jun kinases (JNK) and p38 kinases have been proposed to mediate apoptosis (Verheij et al., 1996; Xia et al., 1995). However, a number of reports have challenged the notion that activation of JNKs and/or p38 is sufficient to induce apoptosis (Lassignal Johnson et al., 1996; Tsubata et al., 1993; Liu et al., 1996a; Juo et al., 1997; Liu et al., 1996b; Park et al., 1996). It appears thus that other signal pathways are required for apoptosis. However, the integration and balance of the JNK and p38 pathways probably does contribute to the commitment to apoptosis (Xia et al., 1995; Gardner and Johnson, 1996).

Several protein serine-threonine kinases referred to as MEK kinases (MEKKs) have been cloned that are members of sequential protein kinase pathways regulating MAP kinases including the c-Jun kinases and ERKs [(Lange-Carter et al., 1993; Lange-Carter and Johnson, 1994; Xu et al., 1996; Blank et al., .1996)]. In our hands, MEKKs do not significantly activate p38 kinases. Of the four MEKK members we have characterized, MEKK1 has been found to have the unique property of being a strong stimulator of apoptosis (Lassignal Johnson et al., 1996; Xia et al., 1995). The other MEKKs, even though they all activate c-Jun kinases and ERKs to different levels, do not induce apoptosis, suggesting MEKK1 has unique substrates that mediate the death response. The kinase domain of MEKK1 is only 50% conserved relative to the kinase domains of MEKK 2, 3 and 4, consistent with MEKK1 having unique substrate recognition properties and catalytic activity involved in mediating the apoptotic response. MEKK1 is a 196 kDa protein that encodes a protease cleavage sequence for CPP32-like proteases. None of the other MEKKs or known kinases that regulate MAPK pathways have a consensus ICE/FLICE cleavage site. We demonstrate in this example that MEKK1 is a substrate for proteases inhibited by the p35 baculovirus protein. When the kinase domain is released from the holo-MEKK1 protein it functions as a physiological activator of apoptosis. UV irradiation and DNA damaging chemicals activate MEKK1 kinase activity and induce its proteolytic cleavage indicating that MEKK1 contributes to apoptosis in response to environmental stresses.

Materials and Methods for this Example:

Cells

Human embryonal kidney 293 cells (HEK293) stably expressing the EBNA-1 protein from Epstein-Barr virus (Invitrogen) were grown in Dulbecco's modified Eagle's medium (DMEM) supplemented with 100 U/ml penicillin/streptomycin and containing 10% bovine calf serum (BCS). The cells were transfected using lipofectamine (Gibco).

Plasmids

The full length cDNA encoding mouse MEKK1 was modified by addition of the HA-tag sequence (MGYPYDVDYAS) (SEQ ID NO: 12) at its NH₂-terminus and inserted into the expression plasmid pCEP4 (Invitrogen), resulting in plasmid MEKK1.cp4. The MEKK1 sequences DTVD (amino acids 871-874) and DEVE (amino acids 857-860) in MEKK1.cp4 were substituted with alanines using a PCR strategy. The resulting plasmids were named DTVD_A.cp4 and DEVE_A.cp4. The cDNAs for CrmA (Pickup et al., 1986), p35 (Cartier et al., 1994), JNK1-APF (Dérijard et al., 1994) and JNK2-APF (Kallunki et al., 1994) were subcloned in pCEP4 in which the hygromycin resistance gene had been removed, resulting in plasmids CrmA.cp_, p35.cp_, JNK1_APF.cp_and JNK2_APF.cp_. Plasmid pCDNA_{—}3.cp4 is the result of the ligation of pCEP4 and pCDNA-3.

In vitro Kinase Assays

Lysis buffer (70 mM β-glycerophosphate, 1 mM EGTA, 100 μM Na₃VO₄, 1 mM DTT, 2 mM MgCl₂, 0.5% Triton-X100, 20 μg/ml aprotinin) was added to cells 15-24 hours after transfection. Cellular debris was removed by centrifugation at 8,000×g for 5 min. Protein concentration was normalized by Bradford assay using BSA as standard.

c-Jun Kinase

c-Jun kinase (JNK) activity was measured using a solid phase kinase assay in which glutathione S-transferase-c-Jun₍₁₋₇₉₎ (GST-Jun) bound to glutathione-Sepharose 4B beads was used to affinity-purify JNK from cell lysates as described (Gardner and Johnson, 1996; Hibi et al., 1993). Alternatively, JNK1 or JNK2 were immunoprecipitated with isoform specific antibodies (Santa Cruz Biotechnology) and GST-Jun used as substrate in an in vitro kinase assay (Hibi et al., 1993). Quantitation of the phosphorylation of GST-Jun was performed with a PhosphorImager.

ERK

ERK2 was immunoprecipitated as described above for the JNK isoforms using the ERK2 (C-14) antibody (Santa Cruz Biotechnology). The beads were washed twice with 1 ml lysis buffer and twice with 1 ml lysis buffer without Triton-X100. Thirty-five μl of the last wash was left in the tube and mixed with 20 μl of kinase 2× mix (50 mM β-glycerophosphate, 100 μM Na₃VO₄, 20 mM MgCl₂, 200 μM ATP, 1 μCi/μl γ³²P-ATP, 400 μM EGF receptor peptide 662-681, 100 μg/μl IP-20, 2 mM EGTA), incubated 20 min at 20° C. and spotted on P81 Whatman paper. The samples were washed thrice for 5 min each in 75 mM phosphoric acid and once for 2 min in acetone, air-dried, and their radioactivity determined in a β counter.

SEK1 K→Phosphorylation

MEKK1 was immunoprecipitated from cell lysates (200-500 μg) with the antibodies raised against specific sequences of MEKK1 or the 12CA5 antibody that recognizes the HA-tag sequence. The immunoprecipitates were used in an in vitro kinase assay with recombinant kinase inactive SEK1 (SEK1 K→M) as previously described (Blank et al., 1996).

MEKK1 Staining and Terminal-deoxy-transferase (TdT)-mediated Incorporation of Fluorescent dUTP

Cells were grown on glass coverslips and transfected using lipofectamine. Two days after transfection, the medium was removed and the cells were fixed in 2% paraformaldehyde, 3% sucrose in phosphate buffered saline (PBS) for 10 min at room temperature. Following three washes with PBS, the cells were permeabilized for 10 min with 2% Triton-X100 in PBS. After three PBS washes, the cells were blocked with filtered cultured medium for 15 min. The coverslips were then incubated 1 hour in TdT reaction mix (200 mM potassium cacodylate, 25 mM Tris.HCl, pH 6.6, 250 μg/ml BSA, 5 mM CoCl₂, 0.25 U/μl TdT [Boehringer], 10 μM biotin-dUTP [Boehringer]) at 37° C. in a humidified atmosphere. After three washes in PBS, the coverslips were incubated for 1 hour at room temperature with a 1/500 dilution in filtered culture medium of an affinity purified rabbit antisera directed at the peptide DRPPSRELLKHPVFR of mouse MEKK1 (amino acids 1476-1490) (Lange-Carter et al., 1993). The coverslips were then washed 6× over a 30 min period with PBS and incubated 1 hour at room temperature with a 1/1000 dilution in filtered culture medium of a donkey anti-rabbit, Cy³-conjugated, antibody (Jackson Immunological) mixed with 5 μg/ml streptavidin conjugated with FITC (Jackson Immunological). The coverslips were washed 6× with PBS and incubated overnight in PBS before being mounted in 20 mg/ml o-phenyldiamine-diHCl (Sigma) in 0.1 M Tris pH 8.5, 90% glycerol. Images were taken using a Leica DMRXA microscope and analyzed with the SlideBook v2.0 software (Intelligent Imaging Innovations, Denver). The subcellular localization of endogenous MEKK1 observed with the anti-COOH-terminal MEKK1 antibody was identical to that observed with a second antibody recognizing the NH₂-terminal portion of the MEKK1 protein.

Immunoblots

200-400 μg cell lysate protein was subjected to SDS-9% PAGE and transferred to nitrocellulose membranes. Blots were performed exactly as described (Widmann et al., 1995). To detect HA-tagged proteins, the mouse monoclonal antibody 12CA5 (Babco) was used as the primary antibody, followed by a rabbit anti-mouse antibody (Cappel). HRP-conjugated protein A at a 1/5000 dilution (Zymed) and ¹²⁵I-protein A at a 1/500 dilution (Dupont NEN) were then used for enhanced chemiluminescence (ECL) detection and for quantification using the PhosphorImager. To detect MEKK1, 3 different polyclonal antisera were used as primary antibodies, followed by ECL detection using HRP-protein A (see above). These sera were generated by injecting rabbits with GST proteins fused with different portions of the MEKK1 protein.

PP-2A Treatment.

MEKK1 was immunoprecipitated from cell lysates (200-500 μg) using the 96-001 (NH₂) antisera, washed twice with 1 ml extraction buffer (BE) [1% Triton-X100; 10 mM Tris pH 7.4; 50 mM Nalco; 50 mM AF; 5 mM EDTA], twice with 1 ml CT (50 mM Tris pH 7.0; 0.1 mM call₂) and once with 1 ml CT containing 60 mM β-mercaptoethanol, 1 mM MgCl₂. 35 μl of the last wash was left in the tube and 0.5 U of PP-2A (Upstate Biotechnology) was added for 30-45 min. The phosphatase reaction was terminated by adding 1 μl of 200 mM Na₃VO₄. For in vitro kinase assay, the immunoprecipitates were washed three more times with 1 ml PAN (10 mM PIPES; 100 mM NaCl; 20 μg/ml aprotinin) before being mixed with the SEK1 K(M substrate and γ³²P-ATP.

### Results

Expression of the 196 kDa MEKK1 Protein by Gene Transfection Induces Apoptosis.

Expression of the 37 kDa kinase domain of MEKK1 (ΔMEKK1) induces cell death by apoptosis (Lassignal Johnson et al., 1996; Xia et al., 1995). To assess whether the full length protein had the same effect, HEK293 cells were transfected with a plasmid encoding the mouse MEKK1 and stained 2 days later for MEKK1 expression using an antibody directed at the COOH-terminus of the protein. To monitor cell death, DNA fragmentation, a feature often associated with apoptosis, was measured by terminal-deoxy-transferase-mediated incorporation of fluorescent dUTP. A large proportion of HEK293 cells expressing MEKK1 had fragmented DNA. The MEKK1 expressing cells characteristically rounded up and began to lift off the coverslips. MEKK1 also induced chromatin condensation and the niuclei in these cells often dissociated from the surrounding cytoplasm. Quantitation of cells exhibiting DNA fragmentation and cells expressing MEKK1 revealed that about 30% of MEKK1-expressing cells were apoptotic after 48 hr. This is an underestimate because the apoptotic cells eventually detach from the coverslips and often loose their nucleus. Thus, expression of the 196 kDa MEKK1 protein by gene transfection induced cell death characteristic of apoptosis similar to that observed for the 37 kDa kinase domain. The kinase activity of MEKK1 is required for the induction of cell death (Lassignal Johnson et al., 1996).

MEKK1-induced DNA Fragmentation is Inhibited by p35 and CrmA.

Inhibition of cysteine proteases of the ICE family by the baculovirus p35 protein or by the poxvirus CrmA protein has been shown to protect cells from apoptosis in response to diverse stimuli (Beidler et al., 1996). Cotransfection of HEK293 cells with MEKK1 and p35 inhibited the DNA fragmentation seen with expression of MEKK1 alone. Cotransfection of MEKK1 with CrmA also inhibited DNA fragmentation, but to a lesser extent. While only about 5% of the cells cotransfected with MEKK1 and p35 showed some DNA fragmentation, this proportion increased to about 15% in MEKK1- and CrmA-cotransfected cells. (Control cells transfected with MEKK1 alone showed. about 30% DNA fragmentation). A small area of fragmented DNA was typically seen in the nucleus of these cells. Thus CrmA appears to be less efficient in protecting cells from MEKK1-induced apoptosis. Interestingly, co-expression of inhibitory mutants of the c-Jun kinases (JNK1-APF and JNK2-APF) with MEKK1 had no or only modest effects on MEKK1-mediated apoptosis. JNK1-APF expression had no effect and JNK2-APF had only a 30% diminution of apoptotic cells induced by MEKK1 expression.

CrmA and p35 Inhibit Cleavage of the 196 kDa MEKK1 Protein and Generation of an Activated Kinase Fragment.

When MEKK1 was expressed by transfection of HEK293 cells, two additional smaller immunoreactive polypeptides besides the full length protein (named A, ˜140 kD, and B, ˜110 kD), were detected by Western blot using an antibody directed to the HA tag of MEKK1 (12CA5 antibody). The 12CA5 antibody recognizes the first 11 amino acids at the NH₂-terminus of the tagged MEKK1 protein, indicating that smaller fragments A and B must be the result of proteolysis of the full length MEKK1 protein and cannot have arisen from other potential translation sites. When an antibody directed at the COOH-terminus of MEKK1 was used (95-012 antibody), additional smaller immunoreactive fragments were also detected. Based on their apparent molecular weight, two of these fragments, named C, ˜90 kD, and D, ˜70 kD, are the corresponding moieties of the cleavage products B and A, respectively. It is also important to note that the proteolytic activity can generate fragment D from fragment C. The observation that MEKK1 can be proteolyzed to very-specific fragments prompted a determination of whether p35 or CrmA could inhibit the generation of fragments A, B, C and D. p35 almost totally, and CrmA partially, inhibited the appearance of fragments B and C. Quantitation of the fragments in 6 independent experiments revealed that CrmA and p35, while leaving the amount of fragment A unchanged, diminished the amount of fragment B by 50% and 90%, respectively. This indicates that these protease inhibitors prevented the formation of fragments B and C, but had no effect on the proteolytic activity that cleaves MEKK1 into fragment A. Since the cleavage of MEKK1 into fragment A was unaffected by CrmA and p35, it was surprising to find that the amount of fragment D, the corresponding moiety of fragment A, was reduced in the presence of the inhibitors. However, because the amounts of fragments A and B formed in MEKK1-transfected cells are not significantly different from one another, the observation that there is far less fragment D than fragment C suggests that fragment D may be unstable and rapidly degraded. Moreover, since fragment D can be derived from fragment C, blocking the generation of fragment C will result in less fragment D. Neither JNK1-APF nor JNK2-APF expression influenced the generation of MEKK1 fragments, suggesting that blunting the activation of the JNK1/JNK2 pathways had little effect on the proteolysis of the MEKK1 protein.

To determine whether the cleavage of MEKK1 into fragments A, B, C and D had any effect on the kinase activity of MEKK1, lysates from cells transfected with HA-tagged MEKK1 alone or in combination with CrmA or p35 were used for immunoprecipitation with the 12CA5 HA antibody or with an antibody specific for the COOH-terminal moiety of MEKK1 (antibody 95-012). The immunoprecipitates were then incubated with a MEKK1 substrate (SEK1 K(M) and γ³²P-ATP. When the full length MEKK1 protein was immunoprecipitated by the 12CA5 antibody it had measurable autophosphorylation and activity towards SEK1. When MEKK1 was immunoprecipitated with the COOH-terminal 95-012 antibody, a stronger SEK1 phosphorylation signal was detected. Since the full length MEKK1 protein and fragments C and D are immunoprecipitated with similar efficiency, the increased phosphorylation of SEK1 was due to the presence of fragments C and D in the immunoprecipitates. This phosphorylation was reduced in the presence of CrmA. In the presence of p35, phosphorylation of SEK1 reached the same level of phosphorylation observed when the 12CA5 antibody was used, that is the basal level of phosphorylation induced by the full length MEKK1. Phosphorylation of fragments C and D was also detected in 95-012 immunoprecipitates. This phosphorylation was reduced by CrmA and almost completely abolished by p35, as expected from the effect of these inhibitors on the generation of fragments C and D. In summary, there is a strong correlation between MEKK1-induced apoptosis and the generation of MEKK1-derived cleavage products that have a stronger kinase activity than the full length protein. This suggests that proteolysis of MEKK1 is involved in the cell death response.

p35 Inhibited Cleavage Occurs at Position Asp⁸⁷⁴ in the Mouse MEKK1 Protein.

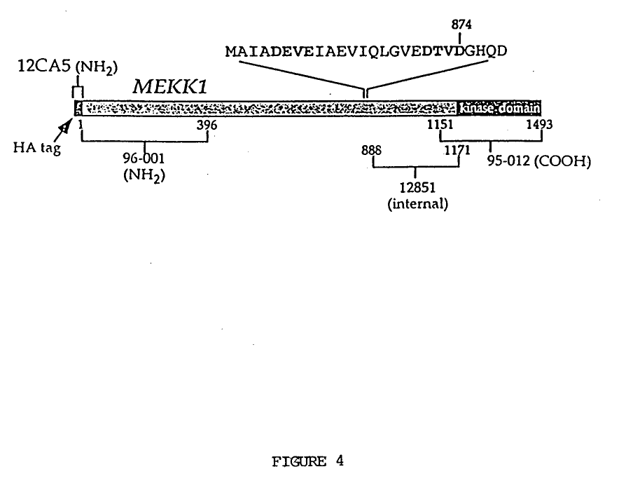

The p35-inhibited cleavage of MEKK1 generates a COOH-terminal fragment of about 90 kDa and a NH₂-terminal fragment of about 110 kDa, indicating that the cleavage occurs between residues 820-900. Two tetrapeptide sequences that are found in this region of MEKK1 closely resemble the CPP32 cleavage site, DEVD (SEQ ID NO: 12) (Nicholson et al., 1995). These sequences are ⁸⁵⁷DEVE⁸⁶⁰ (SEQ ID NO: 7) and ⁸⁷¹DTVD⁸⁷⁴ (SEQ ID NO: 8) (see FIG. 4). The proteases inhibited by p35 have been shown to be cysteine proteases cleaving after the aspartic acid residue in the fourth position of the consensus cleavage sequence (Nicholson et al., 1995; Howard et al., 1991) and, therefore only the DTVD (SEQ ID NO: 8) sequence should be a cleavage site for the CPP32-like protease. Two mutants were generated that have either the DEVE (SEQ ID NO: 7) or the DTVD (SEQ ID NO: 8) sequence replaced with alanine residues (see FIG. 4). These mutants were transfected into HEK293 cells and the presence of MEKK1 and MEKK1-derived fragments were detected by immunoblot analysis using three MEKK1-specific antibodies. When transfected into HEK293 cells, the DEVE→A mutant, like the wild-type protein, was cleaved into fragments A, B, C and D. In contrast, the DTVD→A mutant was only cleaved into fragments A and D. Thus, fragments B and C are not generated in cells expressing the DTVD→A mutant or in cells expressing MEKK1 and p35. This indicates that the p35-inhibited cleavage occurs at position Asp⁸⁷⁴ in the mouse MEKK1 sequence.

The kinase activity of the mutants expressed in HEK293 cells was determined. Immunoprecipitating full length 196 kDa MEKK1 or mutant MEKK1 proteins with the 12CA5 antibody resulted in similar. SEK1 phosphorylating activities. However, when the antibodies directed towards the COOH-terminus of the protein were used, SEK1 phosphorylating activity was reduced in DTVD→A expressing cells as compared to the activity found in wild-type or DEVE→A expressing cells. The reduced kinase activity was comparable to the basal SEK1 phosphorylating activity observed when the full length proteins were immunoprecipitated. Thus, the mutant DTVD→A MEKK1 protein has a low but measurable kinase activity towards SEK1 because fragment C is not generated. The same result was observed when the cleavage of MEKK1 into fragments B and C was inhibited by p35 expression.

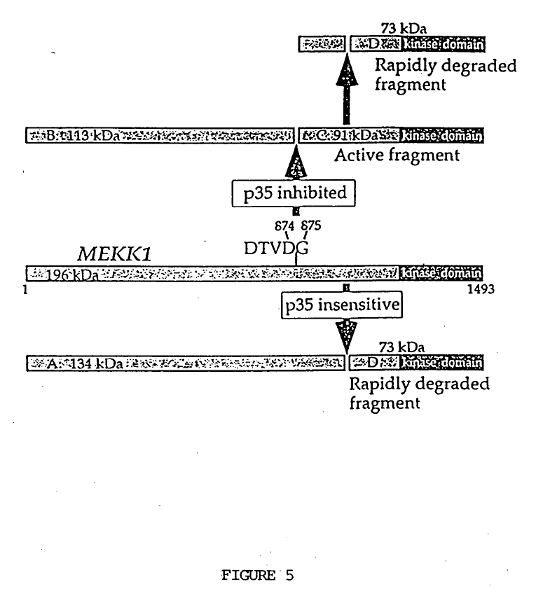

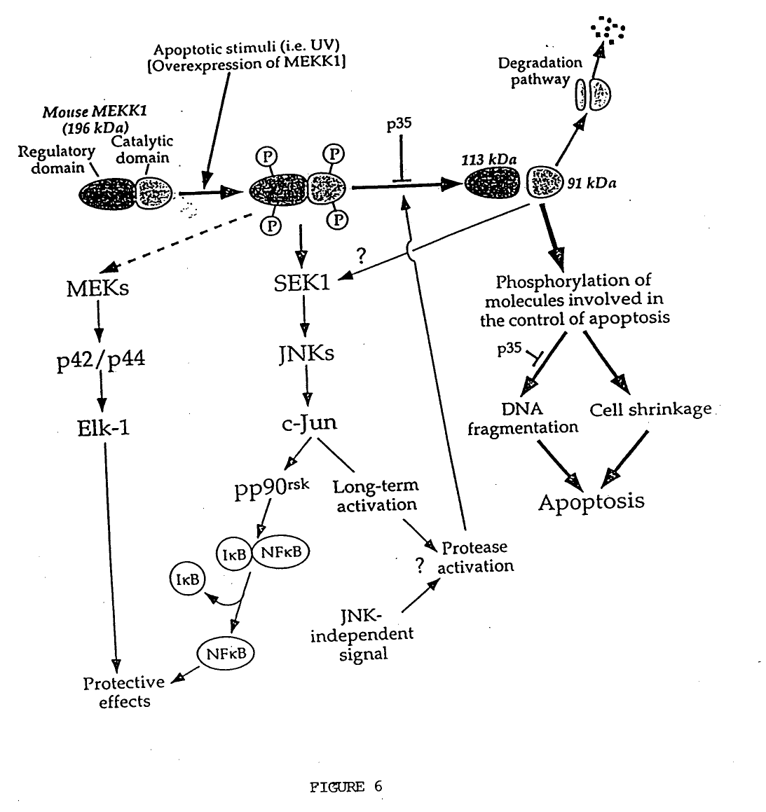

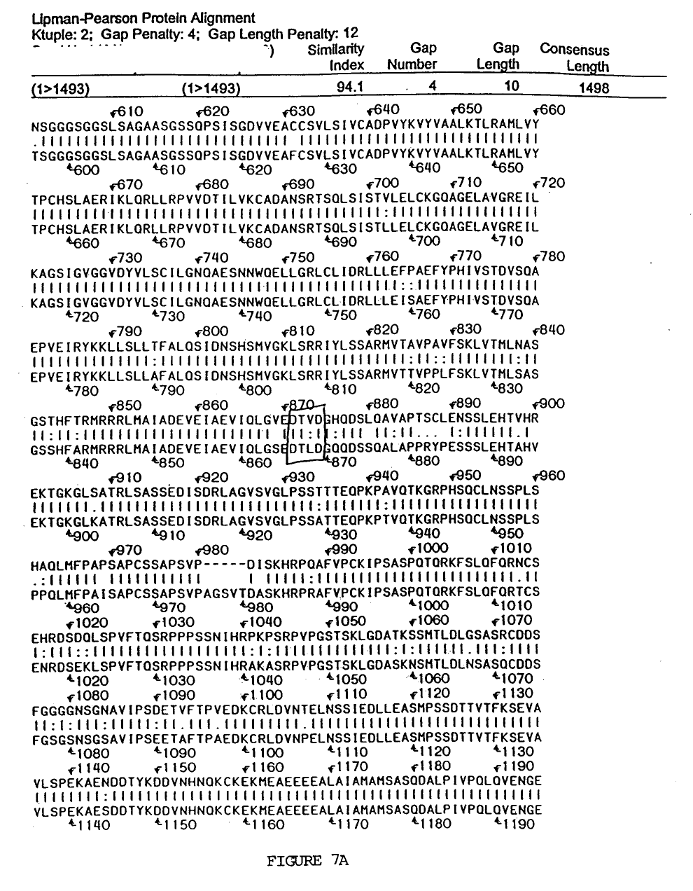

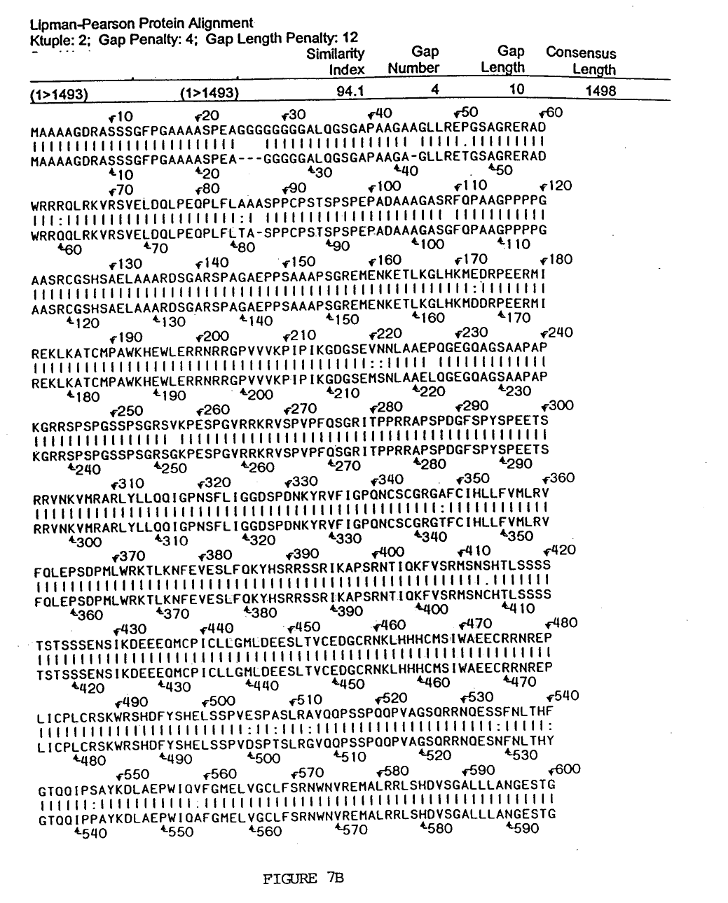

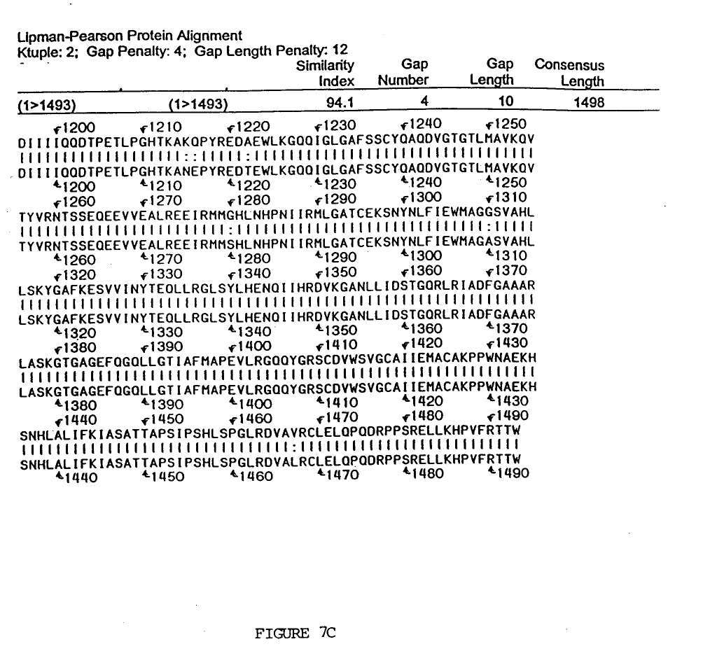

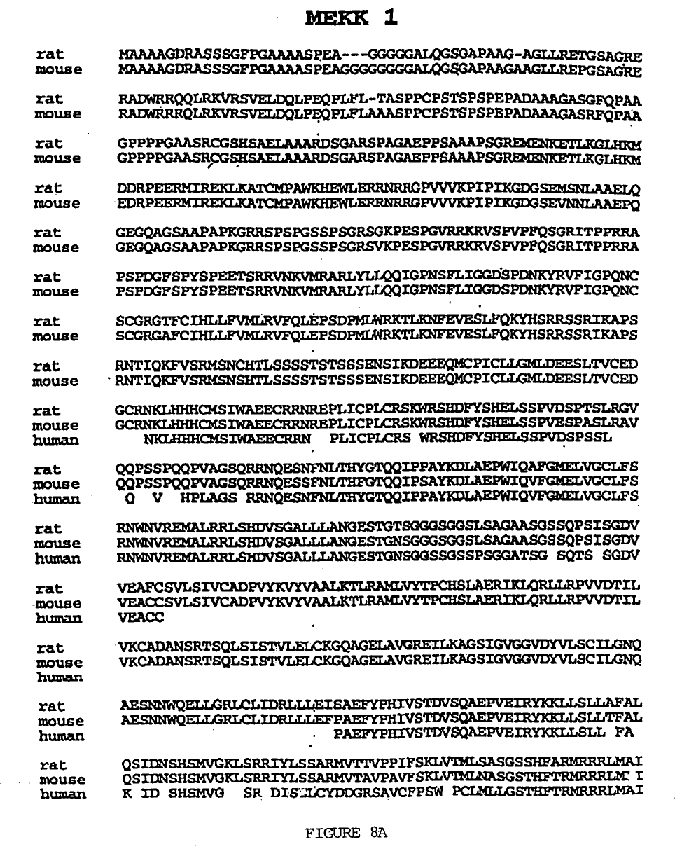

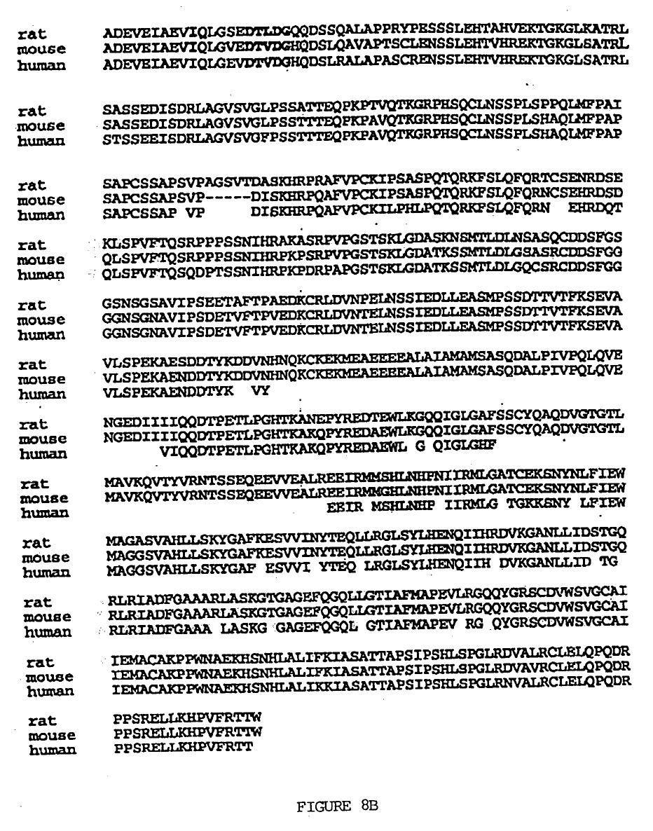

Based on the results described above, FIG. 5 describes a model of the MEKK1 cleavage events occurring in transfected cells. In this model, overexpression of MEKK1 induces deregulated cleavage events generating two sets of fragments (A and D; B and C). Fragment C encoding the catalytic domain of MEKK1 has a stronger kinase activity than the full length protein. Proteases of the ICE/FLICE family are responsible for the cleavage of MEKK1 into fragments B and C because this cleavage can be inhibited by p35 and CrmA. Mutagenesis experiments revealed that the cleavage site generating fragments B and C is DTVD⁸⁷⁴ (SEQ ID NO: 8). Fragment C can be further processed into a smaller polypeptide (fragment D) which may be rapidly degraded. It is possible that the proteolytic activity which generates fragment D is part of a regulatory mechanism involved in the termination of the response induced by cleavage of MEKK1 into the active fragment C.

The DTVD→A Mutant has a Reduced Ability to Promote DNA Fragmentation in HEK293 Cells.

It was next determined whether the DTVD→A mutant induces DNA fragmentation when expressed in HEK293 cells. Expression of the DEVE→A mutant or the wild-type MEKK1 protein induced DNA fragmentation. In contrast, cells expressing the DTVD→A mutant MEKK1 protein showed little DNA fragmentation. Quantitation of the response revealed that the number of DTVD→A expressing cells that showed some DNA fragmentation was reduced by 65% compared to the cells transfected with wild-type MEKK1 or the DEVE→A mutant. This indicates that cleavage of MEKK1 into fragments B and C is required to induce cell death.

p35 Inhibits ΔMEKK1-induced Apoptosis.

The 37 kDa kinase domain of MEKK1 (ΔMEKK1) is a strong inducer of apoptosis (Lassignal Johnson et al., 1996; Xia et al., 1995). Since p35 inhibits programmed cell death induced by most, if not all, apoptotic stimuli (Clem et al., 1996), it was also determined whether this inhibitor could also block ΔMEKK1-induced apoptosis. ΔMEKK1 induced DNA fragmentation when expressed in HEK293 cells. This effect was inhibited by co-expression of p35. Quantitation showed that 40% of cells expressing ΔMEKK1 showed DNA breaks; co-expression of p35 and ΔMEKK1 reduced this number to 10%. The number of ΔMEKK1-expressing cells appeared to be increased when p35 was present, suggesting that less cell death occurred when ΔMEKK1 and p35 were co-expressed. Even if the co-transfected cells showed less DNA fragmentation compared to the cells transfected with ΔMEKK1 alone, they were clearly affected by the expression of ΔMEKK1 and were rounded and most showed some membrane blebbing. This differed from the effect of p35 in full length MEKK1-transfected cells, where the inhibitor appeared to better protect the cells from DNA fragmentation and obvious morphological changes, the predicted result if cleavage of MEKK1 results in the release of an activated kinase domain. These results indicate that p35 inhibits at least two steps in the pathway leading to MEKK1-induced apoptosis, the cleavage of MEKK1 into an active kinase fragment and events downstream of the MEKK1 cleavage that most likely involves a protease step that is influenced by MEKK1.

Activation of the ERK and the JNK Pathways is not Correlated with MEKK1-induced DNA Fragmentation.

MEKK1 activates the ERK and JNK pathways (Xu et al., 1996). Since activation of the JNK pathway has been proposed to induce apoptosis (Verheij et al., 1996), it was next determined whether inhibitory mutants of JNK1 or JNK2 (JNK1-APF and JNK2-APF, respectively) could prevent MEKK1-induced DNA fragmentation. While JNK1-APF had no protective effect, JNK2-APF slightly (by about 30%) reduced the number of MEKK1-expressing apoptotic cells. The competitive inhibitory JNK mutants had no effect on the generation of any cleavage products, indicating that the JNK2-APF-mediated inhibition of MEKK1-induced DNA fragmentation is not related to the cleavage of MEKK1. Activation of ERK2 or the JNKs by MEKK1 was unaffected by the co-expression of JNK1-APF, JNK2-APF, p35 or CrmA. When specific JNK isoforms were immunoprecipitated, only JNK1-APF and JNK2-APF partially inhibited JNK1 and JNK2 activity, respectively. The partial inhibition may be due to cross-reactivity of the antibodies used (Gupta et al., 1996). The DEVE→A and DTVD→A mutants activated JNK to the same level as wild type MEKK1. Transfection of MEKK1 in HEK293 cells did not activate the p38 kinase. Cumulatively, these results show that in conditions where MEKK1-induced DNA fragmentation is inhibited (i.e. when p35 is cotransfected with MEKK1 or when the DTVD→A mutant is expressed), the ERK and the JNK pathways are still activated to an extent similar to that found in MEKK1-transfected cells. This indicates that neither the ERK nor the JNK pathways are sufficient to promote or inhibit the cell death pathway induced by cleavage of MEKK1.

UV Irradiation of HEK293 Cells Induces a Rapid Phosphorylation and Subsequent Cleavage of the Endogenous MEKK1 Protein.

To determine the relevance of these findings in a more physiological situation, the regulation of endogenous MEKK1 in response to UV irradiation, a stress stimulus that induces an apoptotic response, was examined. In HEK293 cells, three different antisera directed at the mouse MEKK1 protein recognized the 196 kDa MEKK1 protein. Several additional nonspecific immunoreactive protein bands were also detected. When cells were treated with UV irradiation (100 J/m²) and incubated for 24 hours in low serum media, the full length MEKK1 protein was no longer detected. Since, we have determined that the half-life of MEKK1 is greater than 24 hours, this result indicates that UV induces a cleavage of the MEKK1 protein. UV irradiation also induced the appearance of new immunoreactive species, the majority of which have molecular weights ranging from about 100 kDa to about 120 kDa. These polypeptides appear thus to be MEKK1-derived fragments generated following MEKK1 proteolysis. The results indicate that UV induces cleavage of the endogenous MEKK1 protein in HEK293 cells.

A time course was performed to determine the effects of UV irradiation on the endogenous MEKK1 protein, activation of the JNK pathway and the extent of apoptosis resulting from the exposure of the cells to a stress stimulus. 15 min after UV irradiation, an MEKK1 species is generated that was upward gel-shifted compared to the MEKK1 species detected before exposure to UV irradiation. One hour after irradiation, most of the full length MEKK1 protein was upward gel-shifted. Eight hours after irradiation, the amount of the gel-shifted MEKK1 started to decrease and 20 hours after UV treatment only a trace amount of full length MEKK1 was detected. The MEKK1 fragment detected by the 96-001 (NH2) antibody was barely seen in the control condition. After 1 hour, however, there was a clear increase in the production of the MEKK1 fragment which reached a maximum 8 hours after UV irradiation. In MEKK1-transfected cells, both the shifted and non-shifted forms of full length MEKK1 were detected. To determine whether the upward gel shift of MEKK1 was due to phosphorylation, lysates of MEKK1-transfected cells were immunoprecipitated with the 12CA5 antibody and incubated with or without protein phosphatase 2A (PP-2A). Phosphatase treatment converted the upper, gel-shifted, form to the lower band, demonstrating that the gel-shift was a phosphorylation-dependent event. To determine whether phosphorylation of MEKK1 was required for its activity, the ability of immunoprecipitated MEKK1 to phosphorylate its substrate SEK1 was assessed after pretreatment with PP-2A. Inmunoprecipitates treated with phosphatase did not phosphorylate SEK1. Thus, phosphorylation of MEKK1 is required for its activation. These results show that UV irradiation induced a rapid phosphorylation of full length MEKK1 followed by its cleavage into fragments. The extent of JNK activation after UV irradiation paralleled the extent of MEKK1 phosphorylation, consistent with the fact that MEKK1 is an upstream regulator of the JNK pathway. Apoptosis, as assessed by morphological changes of the nucleus, started to be detected 8 hours after UV irradiation and was most apparent after 20 hours.

Cleavage of MEKK1 can be Mediated by Different Stress Stimuli.

Several genotoxic stress stimuli were applied to HEK293 cells and their effect on the MEKK1 protein was assessed. UV irradiation, cisplatin, etoposide and mitomycin C induced the loss of full length MEKK1 and the appearance of a lower molecular weight fragment derived from MEKK1. While there was no full length MEKK1 protein remaining after UV and cisplatin treatments, a small amount of upward gel-shifted full length MEKK1 was detected in etoposide and mitomycin C-treated cells. This indicates that chemicals capable of forming DNA adducts, induce the phosphorylation of MEKK1 before its cleavage. These results indicate that the cleavage of MEKK1 may be the activation step leading to apoptosis in a number of stress conditions.

An emerging theme for the cellular commitment to apoptosis involves the activation of specific proteases and the regulation of signal transduction pathways, but the integration of these two regulatory processes in the apoptotic response has not been clearly defined. The role of ICE/FLICE proteases being involved in the apoptotic response is unequivocal (Fraser and Evan, 1996). Loss or inhibition of these enzyme activities can inhibit apoptosis (Los et al., 1995; Darmon and Bleackley, 1996). The notion that signal transduction pathways, specifically those involving the c-Jun kinases and p38 kinases, has developed based on correlative biochemical analysis and gene transfection experiments. An inhibitory mutant of SEK1 (c-Jun kinase kinase) was demonstrated to block ceramide-induced apoptosis in different cell types (Verheij et al., 1996). Similarly, it was shown that a dominant negative c-Jun mutant could block apoptosis of serum-deprived neuronal cells (Xia et al., 1995). Activated mutants of p38 and its immediate upstream regulatory kinase MKK3 was shown to enhance an apoptotic response of PC12 cells to serum deprivation (Xia et al., 1995). The ERK pathway has been shown to have a protective response against an apoptotic stimulus in a few cell types (Xia et al., 1995; Gardner and Johnson, 1996). However, discordance for a role of c-Jun kinases and p38 kinases in mediating apoptosis also exists. For example, MEKK1 mediated apoptosis was shown to be independent of c-Jun kinase activation (Lassignal Johnson et al., 1996). A similar separation of c-Jun kinase activation and apoptosis was observed with the TNF receptor (Liu et al., 1996b).

In this example, it is demonstrated that the JNK pathway is clearly not sufficient to induce the apoptosis mediated by MEKK1. Numerous other examples exist where c-Jun kinase and p38 are activated in response to a stimulus but apoptosis is not observed (Su et al., 1994; Sumimoto et al., 1994; Tsubata et al., 1993). What is however evolving from these studies is that the integration of several different signals, including the regulation of MAP kinase pathways (Xia et al., 1995; Gardner and Johnson, 1996), can contribute to the decision of a cell to commit to apoptosis. Just as with growth and differentiation a series of checkpoints must be overcome before a cell commits itself to death. The needed commitment appears to be activation of the ICE/FLICE protease cascade; activation of c-Jun kinase or p38 pathways may be insufficient by themselves but may enhance or prevent the apoptotic response resulting from an external stimulus such as a genotoxic agent or cytokine.

MEKK1-mediated Apoptosis Requires both Kinase Activity and Proteolytic Cleavage.

We have shown previously that the kinase activity of MEKK1 is required for its apoptotic activity, because the kinase-inactive (MEKK1 is unable to promote apoptosis (Lassignal Johnson et al., 1996). Here it is shown that there is a tight integration of kinase and protease activities in the MEKK1-induced apoptotic pathway. Proteases are required for MEKK1-induced apoptosis at at least two levels in the transduction pathway. The first level corresponds to the cleavage of MEKK1 at position 874 in the mouse MEKK1 sequence. When this cleavage is prevented by the p35 baculovirus protein or when a cleavage-resistant MEKK1 mutant is used, apoptosis is strongly impaired. Proteases of the ICE family of proteases are required for this cleavage to occur, since the viral inhibitors CrmA and p35 inhibit the cleavage. It is indeed likely that CPP32 or a CPP32-like enzyme directly cleaves MEKK1 at position 874, because the recognition site for the protease in the mouse MEKK1 is DTVD, a sequence that closely resembles the DEVD recognition site of the CPP32 substrate poly (ADP-ribose) polymerase (Nicholson et al., 1995). The sequence in the rat MEKK1 sequence that corresponds to the murine DTVD cleavage recognition site is DTLD (Xu et al., 1996); indicating that the cleavage site is conserved between the mouse and the rat MEKK1 proteins and further supports its importance in MEKK1 function. ICE-like proteases are also required at a second step that is downstream of the cleavage of MEKK1 because p35 inhibits the apoptosis induced by the kinase domain of MEKK1.

FIG. 6 shows a model defining the involvement of MEKK1 in apoptosis. The 196 kDa MEKK1 protein can be activated by many extracellular inputs including tyrosine kinase encoded growth factor receptors, G protein-coupled receptors (Avdi et al., 1996) and cellular stresses. Activation of MEKK1 correlates with its phosphorylation. It is unclear at present if MEKK1 phosphorylation involves autophosphorylation or additional kinases. Activated MEKK1 independent of its proteolysis is capable of regulating the c-Jun kinase pathway and may also regulate the ERK pathway. Both of these pathways can stimulate anti-apoptotic responses. Stimulation of the JNK pathway can lead to NFκB activation which is a strong inhibitor of apoptosis (Baeuerle and Baltimore, 1996) and activation of the ERK pathway has been shown to protect cells from apoptosis (Xia et al., 1995; Gardner and Johnson, 1996). With an appropriate protease activation MEKK1 is cleaved to generate a 91 kDa activated kinase domain that has substrates that contribute to driving the cell to apoptosis. Downstream of these phosphorylation events are additional protease substrates that are predicted to be either phosphoproteins or proteins whose activity is regulated by phosphoproteins and which are involved in regulating apoptosis. Bcl-2, for example, would be such a phosphoprotein candidate (Gajewski and Thompson, 1996).

Proteolysis of MEKK1 Generates an Activated Fragment with Altered Cellular Distribution.

It has been demonstrated that the endogenous MEKK1 in resting cells is localized in a post-Golgi vesicular compartment. The punctate cytoplasmic staining of MEKK1 can be seen in non-transfected cells. Upon appropriate cellular stimulation by a growth factor such as EGF MEKK1 is translocated to the plasma membrane. When MEKK1 is overexpressed it is activated and becomes proteolyzed. When MEKK1 is proteolyzed the catalytic domain behaves as a soluble cytoplasmic protein that is no longer sequestered on vesicle-like structures or the plasma membrane. Cleavage of MEKK1 may also change the specificity and activity of the kinase. In vitro kinase assays have indeed revealed that the kinase activity of the cleaved MEKK1 towards SEK1 is increased compared to the full length MEKK1. Thus, the 91 kDa kinase fragment of MEKK1 has a different subcellular distribution from the 196 kDa holo-MEKK1 which may allow it to phosphorylate a different set of substrates.

Genotoxic stress: A Balance Between Rescue and Suicide Using MEKK1 as a Switch.

The results show that DNA damaging chemicals such as cisplatin, etoposide and mitomycin C in addition to UV irradiation induce a phosphorylation correlated with activation of MEKK1. The time course for UV irradiation-induced c-Jun kinase activation closely paralleled that for MEKK1 phosphorylation, consistent with MEKK1 being an upstream regulator of this pathway. Thus, UV irradiation induces a rapid phosphorylation and activation of MEKK1 and c-Jun kinase. The rapid c-Jun kinase response could actually contribute to a protective response against cell death. This has been proposed for the action of CD40 in protecting B cells from antigen crosslinking-induced apoptosis (Sumimoto et al., 1994; Tsubata et al., 1993) and methyl methane sulfonate-induced 3T3 cell apoptosis (Liu et al., 1996a). The activation of NFκB in response to stresses including UV irradiation and genotoxic chemicals would also be a protective response (Baeuerle and Baltimore, 1996); MEKK1 has been shown to be involved in the activation of NFκB (Hirano et al., 1996).

If the stress challenge to the cell is too great a protease cascade is activated involving the ICE/FLICE enzymes (Fraser and Evan, 1996). The data indicate that one substrate for CPP32-like proteases is MEKK1. The time course of MEKK1 proteolysis is slower than its activation; cleavage of MEKK1 releases the 91 kDa kinase domain with new subcellular localization and the ability to activate effectors of apoptosis.

These findings suggest MEKK1 can function as a switch point, regulated by a proteolytic event controlled by ICE/FLICE proteases, that determines cell fate in response to a stress stimulus. Before cleavage MEKK1 induces rescue mechanisms and after cleavage MEKK1 triggers apoptosis. The cleavage of MEKK1 may thus occur when the cell has failed to successfully repair itself. The cleaved MEKK1 then triggers apoptosis which leads to the elimination of the cell.

The above example defines MEKK1 as a protease substrate that when activated and cleaved stimulates an apoptotic response. The proteolytic cleavage of MEKK1 defines the mechanism to generate a protein kinase whose activity is sufficient to induce apoptosis. In the context of cancer therapy, the finding that the activation and cleavage of MEKK1 occurs in response to genotoxic agents is particularly important. For example, expression of MEKK1 is capable of killing by apoptosis cells that have both p53 alleles mutated. Hence, the activation and cleavage of MEKK1 is an apoptotic pathway that does not require a functional p53 and stimulation of these events could enhance the killing of many different tumors. Manipulating the activation of MEKK1 and its cleavage by proteases, with the use of drugs for example, could increase the killing of tumor cells to genotoxic agents. This is consistent with the finding that low level expression of MEKK1 potentiated the apoptotic response to low doses of UV irradiation and cisplatin.

Citations for Publications Referred to in this Example:

An and Dou (1996) Cancer Res. 56, 438-442; Avdi et al: (1996) J. Biol. Chem. 271, 33598-33606; Baeuerle and Baltimore (1996) Cell 87, 13-20; Beidler et al. (1996) J. Biol. Chem. 270, 16526-16528; Blank et al. (1996) J. Biol. Chem. 271, 5361-5368. Canman and Kastan (1996) Nature 384, 213-214; Cartier et al. (1994) J. Virol. 68, 7728-7737; Casciola-Rosen et al. (1994) J. Biol. Chem. 269, 30757-30760; Casciola-Rosen et al. (1995) J. Exp. Med. 182, 1625-1634; Clem et al. (1996) Death Differ. 3, 9-16. Cryns et al. (1996) J. Biol. Chem. 271, 31277-31282; Darmon et al. (1996) J. Biol. Chem. 271, 21699-21702; Dérijard et al. (1994) Cell 76, 1025-1037; Emoto et al. (1995) EMBO J. 14, 6148-6156; Esolen et al. (1995) J. Virol. 69, 3955-3958; Fraser and Evan (1996) Cell 85, 781-784; Gajewski and Thompson (1996) Cell 87, 589-592; Gardner and Johnson (1996) J. Biol. Chem. 271, 14560-14566; Gupta et al. (1996) EMBO J. 15, 2760-2770; Hibi et al. (1993) Genes Develop. 7, 2135-2148; Hinshaw et al. (1994) J. Virol. 68, 3667-3673; Hirano et al. (1996) J. Biol. Chem. 271, 13234-13238; Howard (1991) J. Immunol. 147, 2964-2969.
Juo et al. (1997) Mol. Cell. Biol. 17, 24-35; Kallunki et al. (1994) Genes Develop. 8, 2996-3007; Kägi et al. (1994) Science 265, 528-530; Lange-Carter et al. (1993) Science 260, 315-319; Lange-Carter and Johnson (1994) Science 265, 1458-1461; Lassignal Johnson et al. (1996) J. Biol. Chem. 271, 3229-3237; Lazebnik et al. (1994) Nature 371, 346-347; Lazebnik et al. (1995) Proc. Natl. Acad. Sci. U. S. A. 92, 9042-9046; Liu et al. (1996a) Nature 384, 273-276; Liu et al. (1996b) Cell 87, 565-576; Los et al. (1995) Nature 375, 81-83.
Lowin et al. (1994) Nature 370,650-652; Na et al. (1996) J. Biol. Chem. 271, 11209-11213.
Nicholson et al. (1995) Nature 376, 37-43; Nicoletti et al. (1991) J. Immunol. Methods 139, 271-279; Orth et al. (1996) J. Biol. Chem. 271, 20977-20980; Park et al. (1996) J. Biol. Chem. 271, 21898-21905; Pickup et al. (1986) Proc. Natn. Acad. Sci. U. S. A. 83, 7698-7702; Steller (1995) Science 267, 1445-1449; Su et al. (1994) Cell 77, 727-736; Sumimoto et al. (1994) J. Immunol. 153, 2488-2496; Terai et al. (1991) J. Clin. Invest. 87, 1710-1715; Tsubata et al. (1993) Nature 364, 645-648; Tyler et al. (1995) J. Virol. 69, 6972-6979; Vandenabeele et al. (1995) Trends Cell Biol. 5, 392-399; Verheij et al. (1996) et al. Nature 380, 75-79; Wang et al. (1996) EMBO J. 15, 1012-1020; Widmann et al. (1995) Biochem. J. 310, 203-214; Williams and Smith (1993) Cell 74, 777-779; Xia et al. (1995) Science 270, 1326-1331; and Xu et al. (1996) Proc. Natl. Acad. Sci. U. S. A. 93, 5291-5295.

The foregoing description of the invention has been presented for purposes of illustration and description. Further, the description is not intended to limit the invention to the form disclosed herein. Consequently, variations and modifications commensurate with the above teachings, and the skill or knowledge in the relevant art are within the scope of the present invention. The preferred embodiment described herein above is further intended to explain the best mode known of practicing the invention and to enable others skilled in the art to utilize the invention in various embodiments and with various modifications required by their particular applications or uses of the invention. It is intended that the appended claims be construed to include alternate embodiments to the extent permitted by the prior art. The contents of all references, patents and published patent applications cited throughout this application are hereby incorporated by reference.

1. An isolated nucleic acid molecule comprising the nucleotide sequence of SEQ ID NO:3 or a complement thereof.

2. The nucleic acid molecule of any one of claims 1 and 63-65, further comprising vector nucleic acid sequences.

3. The nucleic acid molecule of any one of claims 1 and 63-65, further comprising nucleic acid sequences encoding a heterologous polypeptide.

4. A host cell which contains the nucleic acid molecule of any one of claims 1 and 63-65.

5. An isolated nucleic acid molecule comprising the nucleotide sequence of SEQ ID NO:5 or a complement thereof.

6. The nucleic acid molecule of claim 5 further comprising vector nucleic acid sequences.

7. The nucleic acid molecule of claim 5 further comprising nucleic acid sequences encoding a heterologous polypeptide.

8. A host cell which contains the nucleic acid molecule of claim 5.

9. An isolated polypeptide selected from the group consisting of:
a) a polypeptide having at least 95% identity to the sequence set forth as SEQ ID NO:4, wherein % identity is determined over the entire length of SEQ ID NO:4 and wherein the protein is capable of phosphorylating a mitogen-activated protein kinase kinase (MKK) protein;
b) the polypeptide of subpart (a), wherein the polypeptide is capable of phosphorylating a MKK protein selected from the group consisting of MKK1, MKK2, MKK3 and MKK4;
c) a polypeptide which is encoded by the nucleic acid molecule comprising the nucleotide sequence of SEQ ID NO:3; and
d) a polypeptide comprising the amino acid sequence of SEQ ID NO:4.

10. A fusion protein comprising the polypeptide of claim 9 operatively linked to heterologous amino acid sequences.

11. An antibody which selectively binds to a polypeptide of claim 9.

12. (canceled)

13. An isolated polypeptide selected from the group consisting of:
a) a polypeptide having at least 95% identity to the sequence set forth as SEQ ID NO:6, wherein % identity is determined over the entire length of SEQ ID NO:6 and wherein the protein is capable of phosphorylating a mitogen-activated protein kinase kinase (MKK) protein;
b) the polypeptide of subpart (a), wherein the polypeptide is capable of phosphorylating a MKK protein selected from the group consisting of MKK1, MKK2, MKK3 and MKK4;
c) a polypeptide which is encoded by the nucleic acid molecule comprising the nucleotide sequence of SEQ ID NO:5; and
d) a polypeptide comprising the amino acid sequence of SEQ ID NO:6.

14. A fusion protein comprising the polypeptide of claim 13 operatively linked to heterologous amino acid sequences.

15. An antibody which selectively binds to a polypeptide of claim 13.

16. (canceled)

17. A method for detecting the presence of a MEKK1 polypeptide in a sample comprising:
a) contacting the sample with a compound which selectively binds to the polypeptide; and
b) determining whether the compound binds to the polypeptide in the sample to thereby detect the presence of a MEKK1 polypeptide in the sample.

18. A kit comprising a compound which selectively binds to a MEKK1 polypeptide and instructions for use.

19. A method for detecting the presence of a MEKK1 nucleic acid molecule in a sample comprising:
a) contacting the sample with a nucleic acid probe or primer which selectively hybridizes to the nucleic acid molecule; and
b) determining whether the nucleic acid probe or primer binds to a nucleic acid molecule in the sample to thereby detect the presence of a MEKK1 nucleic acid molecule in the sample.

20. A kit comprising a compound which selectively hybridizes to a MEKK1 nucleic acid molecule and instructions for use.

21. A method for detecting the presence of a biological activity of a MEKK1 polypeptide in a sample comprising:
a) contacting the sample with an agent capable of detecting MEKK1 activity; and
b) determining the presence of MEKK1 activity in the sample.

22. A method for modulating MEKK1 activity comprising contacting a cell with an agent that modulates MEKK1 activity such that MEKK1 activity in the cell is modulated.

23. The method of claim 22 wherein the agent is selected form the group consisting of an antibody that specifically binds to the MEKK1 protein and a nucleic acid molecule having a nucleotide sequence which is antisense to the coding strand of a MEKK1 mRNA of MEKK1 gene.

24. A method to treat a subject having a disorder characterized by aberrant MEKK1 protein or nucleic acid expression or activity comprising administering an agent which is a MEKK1 modulator to the subject such that MEKK1 protein or nucleic acid expression or activity is modulated.

25. A method for identifying the presence or absence of a genetic alteration characterized by at least one of (i) aberrant modification or mutation of a gene encoding a MEKK1 protein; (ii) mis-regulation of said gene; and (iii) aberrant post-translational modification of a MEKK1 protein, wherein a wild-type form of said gene encodes an protein with a MEKK1 activity

26. An isolated active fragment of an MEKK1 protein consisting of an amino acid sequence having at least 75% homology to an amino acid sequence consisting of about amino acids 875-1493 of SEQ ID NO:4, wherein said active fragment mediates apoptosis.

27. The active fragment of claim 26, which consists of an amino acid sequence having at least 85% homology to an amino acid sequence consisting of about amino acids 875-1493 of SEQ ID NO:4.

28. The active fragment of claim 26, which consists of an amino acid sequence having at least 95% homology to an amino acid sequence consisting of about amino acids 875-1493 of FIG. 9.

29. (canceled)

30. (canceled)

31. (canceled)

32. The active fragment of claim 26, which consists of about amino acids 875-1493 of SEQ ID NO:4.

33. The active fragment of claim 26, which consists of about amino acids 685-1303 of SEQ ID NO:6.

34. An isolated protease-resistant MEKK1 protein comprising an amino acid sequence having at least 75% homology to the amino acid sequence of SEQ ID NO:4, wherein at least one amino acid equivalent to amino acids 871-874 of SEQ ID NO:4 is substituted such that the MEKK1 protein is resistant to proteolysis by a caspase after amino acid 874.

35. The MEKK1 protein of claim 34, wherein at least one amino acid equivalent to amino acids 871-874 of SEQ ID NO:4 is substituted with an alanine residue.

36. The MEKK1 protein of claim 34, wherein each amino acid equivalent to amino acids 871-874 of SEQ ID NO:4 is substituted with an alanine residue.

37. The MEKK1 protein of claim 34, which has at least 85% homology to the amino acid sequence of SEQ ID NO:4.

38. The MEKK1 protein of claim 34, which has at least 95% homology to the amino acid sequence of SEQ ID NO:4.

39. (canceled)

40. (canceled)

41. The MEKK protein of claim 40 consisting of amino acids 685-1303 of SEQ ID NO:6.

42. (canceled)

43. An isolated nucleic acid molecule which encodes an active fragment of MEKK1 that mediates apoptosis, said fragment having 95% sequence identity to residues 875-1493 of SEQ ID NO:4, wherein % identity is determined over the entire length of residues 875-1493 of SEQ ID NO:4.

44-48. (canceled)

49. The nucleic acid molecule of claim 43, which consists of about nucleotides 2637-4493 of SEQ ID NO:3, or a nucleotide sequence that, due to the degeneracy of the genetic code, encodes amino acid residues 875-1493 of SEQ ID NO:4.

50. The nucleic acid molecule of claim 43, which consists of nucleotides 2637-4493 of SEQ ID NO:3, or a nucleotide sequence that, due to the degeneracy of the genetic code, encodes amino acid residues 875-1493 of SEQ ID NO:4.

51. The nucleic acid molecule of claim 43, which consists of nucleotides 2052-3908 of SEQ ID NO:5, or a nucleotide sequence that, due to the degeneracy of the genetic code, encodes amino acid residues 685-1303 of SEQ ID NO:6.

52. An isolated nucleic acid molecule encoding a protease-resistant MEKK1 protein, wherein the protease resistant MEKK1 protein comprises an amino acid sequence having at least 95% identity to the amino acid sequence of SEQ ID NO:4, wherein % identity is determined over the entire length of SEQ ID NO:4, and wherein at least one codon of the nucleic acid molecule encoding an amino acid equivalent to at least one of amino acids 871-874 of SEQ ID NO:4 is mutated such the encoded MEKK1 protein is resistant to proteolysis by a caspase after an amino acid equivalent to amino acid 874 of SEQ ID NO:4.

53. The nucleic acid molecule of claim 52, wherein at least one codon is mutated to encode an alanine residue.

54. The nucleic acid molecule of claim 52, wherein each codon is mutated to encode an alanine residue.

55-57. (canceled)

58. An expression vector comprising the nucleic acid molecule of claim 43.

59. An expression vector comprising the nucleic acid molecule of claim 52.

60. A host cell containing the expression vector of claim 58.

61. A host cell containing the expression vector of claim 59.

62. An isolated nucleic acid molecule encoding a protease-resistant MEKK1 protein, wherein the protease resistant MEKK1 protein comprises the amino acid sequence of SEQ ID NO:6 and at least one codon of the nucleic acid molecule encoding an amino acid equivalent to at least one of amino acids 681-684 of SEQ ID NO:6 is mutated such the encoded MEKK1 protein is resistant to proteolysis by a caspase after an amino acid equivalent to amino acid 681-684 of SEQ ID NO:6.

63. A method of stimulating apoptosis in a cell comprising introducing into the cell an expression vector encoding a MEKK1 active fragment such that MEKK1 active fragment is produced in the cell and apoptosis is stimulated.

64. A method of inhibiting apoptosis in a cell comprising introducing into the cell an expression vector encoding a protease-resistant MEKK1 protein such that protease-resistant MEKK1 protein is produced in the cell and apoptosis is inhibited.

65. A method of generating an MEKK 1 active fragment in vitro, comprising:
contacting an MEKK1 protein in vitro with a caspase protease under proteolysis conditions; and
allowing the caspase protease to cleave the MEKK1 protein such that an MEKK1 active fragment is generated.

66. A method of identifying a compound that modulates the apoptotic activity of an MEKK1 active fragment, comprising:
providing an indicator cell that comprises a MEKK1 active fragment;
contacting the indicator cell with a test compound; and
determining the effect of the test compound on the apoptotic activity of the MEKK1 active fragment in the indicator cell to thereby identify a compound that modulates the apoptotic activity of the MEKK1 active fragment.

67. A method of identifying a compound that modulates the proteolytic cleavage of an MEKK1 protein by a caspase protease, comprising:
providing a reaction mixture that comprises an MEKK1 protein and a caspase protease;
contacting the reaction mixture with a test compound; and
determining the effect of the test compound on proteolytic cleavage of the MEKK1 protein by the caspase protease to thereby identify a compound that modulates the proteolytic cleavage of an MEKK1 protein by a caspase protease.

68. An isolated nucleic acid molecule which encodes a protein having at least 95% identity to the sequence set forth as SEQ ID NO:4, wherein % identity is determined over the entire length of SEQ ID NO:4 and wherein the protein is capable of phosphorylating a mitogen-activated protein kinase kinase (MKK) protein.

69. The nucleic acid molecule of claim 63, wherein the encoded protein is capable of phosphorylating a MKK protein selected from the group consisting of MKK1, MKK2, MKK3 and MKK4.

70. An isolated nucleic acid molecule which encodes a protein comprising the amino acid sequence of SEQ ID NO:4.

71. The isolated nucleic acid molecule of claim 52, which encodes the amino acid sequence of SEQ ID NO:4 but for the at least one codon mutation.
